(19) United States
(12) Patent Application Publication
(10) Pub. No.: US 2009/0123626 A1
(43) Pub. Date: May 14, 2009

(54) REDUCED ACRYLAMIDE PLANTS AND FOODS

(75) Inventors: Caius Rommens, Boise, ID (US); Hua Yan, Boise, ID (US); Jingsong Ye, Boise, ID (US)

Correspondence Address: FOLEY AND LARDNER LLP;SUITE 500, 3000 K STREET NW, WASHINGTON, DC, 20007, US

(73) Assignee: J.R. SIMPLOT COMPANY

(21) Appl. No.: 12/237,883

(22) Filed: Sep. 25, 2008

Related U.S. Application Data
(60) Provisional application No. 60/976,042, filed on Sep. 28, 2007.
(60) Provisional application No. 60/978,027, filed on Oct. 5, 2007.

(51) Int. Cl. A21D 13/02; C12N 15/82; A23L 1/216; A23F 5/00

(52) U.S. Cl. 426/549; 435/468; 426/637; 426/595

(57) ABSTRACT

The present invention relates to genetic methods for down-regulating and up-regulating genes in a plant, for example in the starch-rich storage organs of these plants, as well as the products of these plants, to lower the level of acrylamide that accumulates upon processing-associated heating of these organs and plant products.

This U.S. Non-Provisional Application claims priority to U.S. Provisional Application Ser. Nos. 60/976,042, filed on Sep. 28, 2007, and 60/978,027, filed on Oct. 5, 2007, the entireties of which are both incorporated herein by reference.

### FIELD OF THE INVENTION

The present invention relates to genetic methods for down-regulating and up-regulating genes in a plant, for example in the starch-rich storage organs of these plants, to lower the level of acrylamide that accumulates upon processing-associated heating of these organs.

### BACKGROUND

Dietary intake levels of acrylamide have been rising in the Western world since the early 1900s and are currently estimated at 40 μg/person/day. Acrylamide is formed in starchy foods when they are baked or fried, basically as a consequence of the reaction between certain sugars and the free amino acid asparagine. This reactive compound is present in products derived from wheat flour, coffee beans, and potato tubers. Ingested acrylamide is readily absorbed and, in part, metabolized by a cytochrome P450 to produce mercapturic acid and glycidamide. Whereas mercapturic acid is excreted via urine, both the remaining acrylamide and its reactive metabolite bind to various proteins as well as DNA. High levels of adduct formation have been linked to animal health issues including cumulative nerve terminal damage. As suggested by several recent studies, acrylamide intake may also be associated with the increased incidence of neurodegenerative diseases and cancer.

Recently developed methods that limit acrylamide formation to some extent require changes in grower or processor practices. Such modifications often limit the applicability of the method and/or affect the quality of the final product. For instance, the beneficial effect of sulfur fertilization on lowering the acrylamide potential of potato and wheat is off-set by increased farmer input costs and sulfur contamination issues. Furthermore, the partial reductions in acrylamide concentration that can be achieved by modifying processing variables, such as the time and temperature of heating, yields products that lost some of their original sensory characteristics. A third approach incubates raw materials with either asparagine-metabolizing enzymes or amino acids that compete with asparagine in the Maillard reaction. Such treatments were shown to be only partially effective for some raw food ingredients, require high concentrations of the additive, and are also too difficult and costly to apply broadly.

### SUMMARY OF THE INVENTION

In one embodiment, the invention provides a strategy for reducing the levels of acrylamide in a processed food that was obtained by heating the starchy tissues of a crop. This strategy employs various methods in genetic engineering that reduce the levels of asparagine in the starchy tissues of a crop plant by at least 50%.

In one embodiment, the levels of asparagine in the starchy tissues of a crop plant are reduced by modifying the expression of at least one gene that is involved in the accumulation of an asparagine precursor.

In a first aspect, the gene that is involved in the accumulation of an asparagine precursor is upregulated in expression and encodes a glutamine-rich storage protein, which is a storage protein consisting of at least 10% glutamine. In one aspect, a glutamine-rich storage protein that consists of at least 10% glutamine is a wheat glutenin.

In a second aspect, the gene that is involved in the accumulation of an asparagine precursor is down-regulated in expression and encodes a glutamine synthetase.

In a third aspect, the gene that is involved in the accumulation of an asparagine precursor is down-regulated in expression and encodes a mitochondrial malate dehydrogenase.

In a fourth aspect, the gene that is indirectly involved in the biosynthesis of asparagine is down-regulated in expression and encodes an aspartate aminotransferase. In one aspect, the gene involved in aspartate metabolism encodes a lysine feed-back less-sensitive form of aspartate kinase.

In a fifth aspect, the gene that is indirectly involved in the biosynthesis of asparagine is up-regulated in expression and encodes a lysine-feedback-less-sensitive dihydrodipicolinate synthase

In one aspect, lowered expression of a gene that is involved in the accumulation of an asparagine precursor is accomplished by introducing into a plant an expression cassette comprising, from 5′ to 3′, (i) a promoter, (ii) at least one copy of a sequence comprising at least a fragment of at least one gene that is involved in the accumulation of an asparagine precursor, and optionally, (iii) either a second promoter or a terminator, whereby the first and optional second promoter are positioned in the convergent orientation.

A fragment of a gene or polynucleotide may comprise about 5 contiguous nucleotides, about 6 contiguous nucleotides, about 20 contiguous nucleotides, about 21 contiguous nucleotides, about 22 contiguous nucleotides, about 23 contiguous nucleotides, about 24 contiguous nucleotides, about 25 contiguous nucleotides, about 26 contiguous nucleotides, about 27 contiguous nucleotides, about 28 contiguous nucleotides, about 29 contiguous nucleotides, about 30 contiguous nucleotides, about 31 contiguous nucleotides, about 32 contiguous nucleotides, about 33 contiguous nucleotides, about 34 contiguous nucleotides, about 35 contiguous nucleotides, about 36 contiguous nucleotides, about 37 contiguous nucleotides, about 38 contiguous nucleotides, about 39 contiguous nucleotides, about 40 contiguous nucleotides, about 41 contiguous nucleotides, about 42 contiguous nucleotides, about 43 contiguous nucleotides, about 44 contiguous nucleotides, about 45 contiguous nucleotides, about 46 contiguous nucleotides, about 47 contiguous nucleotides, about 48 contiguous nucleotides, about 49 contiguous nucleotides, about 50 contiguous nucleotides, about 60 contiguous nucleotides, about 70 contiguous nucleotides, about 80 contiguous nucleotides, about 90 contiguous nucleotides, about 100 contiguous nucleotides, about 150 contiguous nucleotides, about 200 contiguous nucleotides, about 250 contiguous nucleotides, about 300 contiguous nucleotides, or more than about 300 contiguous nucleotides.

In one aspect, the expression cassette that is used to lower expression of a gene that is involved in the accumulation of an asparagine precursor contains two copies of a sequence comprising at least a fragment of this gene.

In one aspect, the two copies are positioned as (i) inverted repeat, or (ii) direct repeat.

In one aspect, the gene that is involved in the accumulation of an asparagine precursor is isolated from potato, a wild potato such as Solanum phureja, sweet potato, yam, coffee tree, cocoa tree, wheat, maize, oats, sorghum, or barley, and is selected from the group consisting of a glutamine synthetase gene, an aspartate aminotransferase gene, and a mitochondrial malate dehydrogenase gene.

In one aspect the aspartate aminotransferase gene encodes a cytoplasmic protein and comprises a sequence that shares at least 70% identity with at least a fragment of the sequence shown in SEQ ID.:1-5.

In one aspect, overexpression of a gene that is involved in the accumulation of an asparagine precursor is accomplished by introducing into a plant an expression cassette comprising, from 5′ to 3′, a first promoter, a gene that is involved in the accumulation of an asparagine precursor, and a terminator.

In one aspect, the gene that is involved in the accumulation of an asparagine precursor is isolated from potato, a wild potato such as Solanum phureja, sweet potato, yam, coffee tree, cocoa tree, wheat, maize, oats, sorghum, or barley, and is selected from the group consisting of a gene encoding a lysine feed-back less-sensitive form of aspartate kinase, a gene encoding a lysine feed-back less-sensitive form of dihydrodipicolinate synthase, and a glutamine-rich storage protein gene.

In one aspect the lysine feed-back less-sensitive form aspartate kinase gene comprises a sequence that shares at least 70% identity with the sequence shown in SEQ ID.: 14-18.

In one aspect, the promoter is a promoter of (i) a potato granule bound starch synthase gene, (ii) a potato ADP glucose pyrophosphorylase gene, (iii) a potato ubiquitin-7 gene, (iv) a potato patatin gene, (v) a potato flavonoid mono-oxygenase gene.

In one aspect, the promoter of a potato granule bound starch synthase gene shares at least 70% identity with at least part of SEQ ID NO: 12, the promoter of a potato ADP glucose pyrophosphorylase gene shares at least 70% identity with at least part of SEQ ID NO: 11, and a promoter of a potato flavonoid mono-oxygenase gene shares at least 70% identity with at least part of SEQ ID NO: 19.

In another aspect the promoter is the promoter of a gene that is expressed in a tuber, root, or seed of a starchy crop destined for food processing.

In another embodiment, the invention provides a method for reducing the levels of acrylamide in a food that was obtained by heating the tissues of a crop by simultaneously reducing the levels of both an asparagine precursor and reducing-sugars in the tissues. In one embodiment, the tissue is a starchy tissue of the crop or plant.

In one aspect, the simultaneous reduction in levels of both an asparagine precursor and reducing-sugars is obtained by (i) either downregulating the expression of a gene involved in aspartate biosynthesis or overexpressing a gene involved in aspartate metabolism, and (ii) downregulating the expression of at least one gene involved starch degradation.

In one aspect, the expression of a gene in starch degradation is downregulated by introducing into a plant an expression cassette comprising, from 5′ to 3′, (i) a promoter, (ii) at least one copy of a sequence comprising at least a fragment of a gene involved in starch degradation, and optionally, (iii) either a second promoter or a terminator, whereby the first and optional second promoter are positioned in the convergent orientation.

In one aspect, a gene involved in starch degradation is selected from the group consisting of (i) a starch-associated R1 gene, and (ii) a starch-associated phosphorylase-L gene.

In another embodiment, the invention provides a method for simultaneously reducing the levels of acrylamide in a food that was obtained by heating the tissues of a crop and increasing the sensory characteristics of this food by simultaneously reducing the levels of both an asparagine precursor and reducing-sugars in the tissues. In one embodiment, the tissue is a starchy tissue of the crop or plant.

In one aspect, simultaneous reduction is accomplished by employing a ‘multigene-targeting’ construct comprising a first expression cassette comprising either (i) a lysine feed-back less-sensitive form of aspartate kinase gene operably linked to a promoter or (ii) at least one copy of a fragment of an aspartate aminotransferase gene operably linked to a promoter and a second expression cassette comprising at least one copy of a DNA segment comprising both a fragment of the R1 gene and a fragment of the phosphorylase-L gene operably linked to a promoter, whereby the first and second expression cassette can be the same expression cassette.

In one embodiment, a plant comprises at least one cell stably transformed with the ‘multigene-targeting’ construct. In a further embodiment, the plant is tuber-bearing. In another embodiment, the tuber-bearing plant is a potato plant. In another embodiment, the tuber-bearing plant contains the cassette stably integrated into its genome.

In another aspect, the invention provides a processed product from a transgenic tuber, wherein (a) at least one cell of the transgenic tuber comprises the ‘multigene-targeting’ construct, and (b) the product has a lower concentration of acrylamide than an equivalent product from a non-transgenic tuber of the same plant variety.

In another aspect, the invention provides a product from a transgenic tuber, wherein (a) at least one cell of the transgenic tuber comprises the ‘multigene-targeting’ construct, (b) the product has a lower concentration of acrylamide than an equivalent product from a non-transgenic tuber of the same species, and (c) the product further exhibits a lower rate of non-enzymatic browning compared to the equivalent product from the non-transgenic tuber of the same species.

In another aspect, the invention provides a product from a transgenic tuber, wherein (a) at least one cell of the transgenic tuber comprises the ‘multigene-targeting’ construct, (b) the product has a lower concentration of acrylamide than an equivalent product from a non-transgenic tuber of the same species, and (c) the product further exhibits a lower rate of non-enzymatic browning compared to the equivalent product from the non-transgenic tuber of the same species, and (d) the product further displays an enhanced sensory profile compared to the equivalent product from the non-transgenic tuber of the same species. In one embodiment, the edible plant product has improved sensory characteristics compared to an equivalent product from a plant that has not been modified according to the present inventive methods. In one embodiment, the edible plant product has at least one improved sensory characteristic selected from the group consisting of appearance, flavor, aroma, and texture.

In one embodiment, the transgenic tuber is a potato. In a further embodiment, the product is a French fry. In another further embodiment, the product is a chip.

In another embodiment, the transgenic tuber is a potato and the product of the transgenic tuber when stored between 4° C. and 12° C. for about one to thirty weeks contains a glucose level that is less than 50% of the glucose level of a non-transgenic tuber of the same species stored under the same storage conditions as the transgenic tuber.

In one embodiment, the plant is selected from the group consisting of potato, corn, coffee, cocoa, and wheat.

In another embodiment, the plant is transformed with a bacterium strain selected from the group consisting of Agrobacterium tumefaciens, Rhizobium trifolii, Rhizobium leguminosarum, Phyllobacterium myrsinacearum, SinoRhizobium meliloti, and MesoRhizobium loti.

In one aspect, the invention provides an isolated polynucleotide sequence comprising a nucleic acid sequence that codes for a polypeptide that is capable of reducing aspartate levels in a plant and, consequently, reducing acrylamide levels in the processed product of this plant.

In one embodiment, the nucleic acid sequence shares at least 70% identity with SEQ ID NO: 14, 16, 17, or 18, or a variant or fragment thereof and said nucleic acid encodes a polypeptide having aspartate kinase activity.

In a further embodiment, the variant has a sequence identity that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% in sequence to SEQ ID NO: 14, 16, 17, or 18.

In a further embodiment, the variant has a sequence identity that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% in sequence to any sequence disclosed herein.

In another aspect, there is provided an isolated polynucleotide sequence comprising a nucleic acid sequence encoding a lysine feed-back less-sensitive form of aspartate kinase that is capable of reducing aspartate and thereby asparagine levels in a plant and, consequently, reducing acrylamide levels in the processed product of this plant.

In another aspect, the invention provides a transgenic plant having reduced aspartate aminotransferase expression levels, which can be used to produce a processed food containing a reduced acrylamide content.

In another aspect, the invention provides a food having reduced acrylamide content that was obtained through processing a transgenic tuber having reduced asparagine content.

Based on U.S. Food an Drug Administration data (www.cfsan.fda.gov/˜dms/acrydata.html), a typical French fry produced at a restaurant of a large fast food chain contains more than 100 parts-per-billion (ppb) acrylamide. The average amount of acrylamide in such a typical French fry is 404 ppb, and the average daily intake levels of acrylamide through consumption of French fries is 0.07 microgram/kilogram of bodyweight/day. Consequently, French fries represent 16% of the total dietary intake of acrylamide. The average amount of acrylamide in oven-baked French fries and potato chips produced by a commercial processor are 698 ppb and 597 ppb, respectively. Thus, potato-derived processed foods including French fries, over-baked fries, and potato chips represent 38% of the total dietary intake for acrylamide.

According to the present invention, the level of acrylamide that is present in a French fry, baked fry, or chip that is obtained from a tuber produced by a transgenic plant of a specific variety of the present invention is lower than the level of acrylamide in a French fry, baked fry, or chip that is obtained from a non-transgenic plant of the same variety by greater than 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more than 20-fold.

In terms of parts per billion, a French fry produced from a tuber of the present invention may have between 1-20 ppb, 20-40 ppb, 40-60 ppb, 60-80 ppb, or 80-100 ppb acrylamide.

In terms of parts per billion, a oven-baked fry, potato chip, or hash brown produced from a tuber of the present invention may have between 1-20 ppb, 20-40 ppb, 40-60 ppb, 60-80 ppb, 80-100 ppb, 100-120 ppb, 120-140 ppb, 140-160 ppb, 160-180 ppb, or 180-200 ppb acrylamide.

The present invention is not limited to reducing the level of acrylamide in Fry and chip products of tubers. Other foodstuffs may be manipulated according to the present invention to reduce acrylamide levels.

The levels of acrylamide in breakfast cereals can be reduced from about 50-250 ppb to levels below 40 ppb, preferably to levels below 20 ppb.

The levels of acrylamide in crackers such as Dare Breton Thin Wheat Crackers and Wasa Original Crispbread Fiber Rye can be reduced from about 300-500 ppb to levels below 100 ppb, preferably to levels below 50 ppb.

The levels of acrylamide in chocolate such as Ghirardelli Unsweetened Cocoa or Hershey's Cocoa can be reduced from about 300-900 ppb to levels below 200 ppb, preferably to levels below 50 ppb.

The levels of acrylamide in cookies can be reduced from about 50-200 ppb to levels below 40 ppb, preferably to levels below 20 ppb.

The levels of acrylamide in ground coffee can be reduced from about 175-350 ppb to levels below 60 ppb, preferably to levels below 30 ppb.

The levels of acrylamide in wheat bread can be reduced from about 50-150 ppb to levels below 30 ppb, preferably to levels below 15 ppb.

It should be understood that many factors influence the levels of acrylamide in a final food product. Such factors include crop, variety, growing conditions, storage conditions of the harvested seed or tuber, and processing variables such as heating temperature, heating time, type of oil used for frying, and exposed surface.

Application of the methods described in the present invention will lower acrylamide levels by at least about 5%, at least about 10%, at least about 15%, at least about 20%, at least about 25%, at least about 30%, at least about 35%, at least about 40%, at least about 45%, 50%, by at least about 60%, by at least about 70%, by at least about 80%, by at least about 90% or by more than about 90%.

Thus, one aspect of the present invention is a method for reducing acrylamide levels in the heat-processed product of a plant, comprising reducing the levels of asparagine in cells of the plant. In one embodiment, the levels of asparagine are reduced by modifying the expression of at least one target gene that is involved in the accumulation of asparagine.

In one embodiment, the step of modifying the expression of a target gene comprises upregulating the expression of a target gene selected from the group consisting of (i) a glutamine-rich storage protein gene, and (ii) a lysine-feedback-less-sensitive dihydrodipicolinate synthase gene. In one embodiment, the glutamine-rich storage protein target gene encodes a wheat glutenin.

In one embodiment, the step of upregulating the expression of a target gene comprises introducing into cells of the plant an expression cassette which comprises one or more copies of a target gene sequence fragment operably linked to at least one promoter, wherein expression of the target gene fragment(s) produces at least one of a single-stranded RNA transcript or a double-stranded RNA transcript. In one embodiment, the target gene sequence fragment is (i) operably linked to a first promoter at the 5′-end of the fragment, (ii) operably linked to a second promoter at the 3′-end of the fragment, but wherein the fragment (ii) is not operably linked to a terminator. In a further embodiment, the target gene sequence fragment comprises at least about 20-50 contiguous nucleotides of a sequence of the target gene. In another embodiment, the expression cassette comprises at least two copies of the target gene sequence fragment. In one embodiment, the copies of the target gene sequence fragment are oriented in the expression cassette as (i) inverted repeats, or (ii) direct repeats. In a further embodiment, the inverted repeat sequence, or the direct repeat sequence, is (i) operably linked to a first promoter at the 5′-end of the repeat sequence, (ii) operably linked to a second promoter at the 3′-end of the repeat sequence, but wherein the repeat sequence (ii) is not operably linked to a terminator.

In another embodiment, the step of modifying the expression of a target gene comprises downregulating the expression of a target gene selected from the group consisting of (i) a glutamine synthetase gene, (ii) a mitochondrial malate dehydrogenase gene, (iii) an aspartate aminotransferase, and (iv) a lysine feed-back less-sensitive form of an aspartate kinase gene.

In one embodiment, the step of downregulating the expression of a gene comprises introducing into cells of the plant an expression cassette which comprises one or more copies of a target gene sequence fragment operably linked to at least one promoter, wherein expression of the target gene fragment(s) produces at least one of a single-stranded RNA transcript or a double-stranded RNA transcript. In one embodiment, the target gene sequence fragment is (i) operably linked to a first promoter at the 5′-end of the fragment, (ii) operably linked to a second promoter at the 3′-end of the fragment, but wherein the fragment (ii) is not operably linked to a terminator. In another embodiment, the target gene sequence fragment comprises at least about 20-50 contiguous nucleotides of a sequence of the target gene. In a further embodiment, the expression cassette comprises at least two copies of the target gene sequence fragment. In one embodiment, the copies of the target gene sequence fragment are oriented in the expression cassette as (i) inverted repeats, or (ii) direct repeats. In this embodiment, the inverted repeat sequence, or the direct repeat sequence, is (i) operably linked to a first promoter at the 5′-end of the repeat sequence, (ii) operably linked to a second promoter at the 3′-end of the repeat sequence, but wherein the repeat sequence (ii) is not operably linked to a terminator. In another embodiment, the target gene encodes aspartate aminotransferase, and wherein the target gene sequence fragment shares at least 70% identity with a sequence depicted in any one of SEQ ID NOs: 1, 2, 3, 4, or 5.

In another embodiment, the target gene encodes a lysine feed-back less-sensitive form aspartate kinase, and wherein the target gene sequence fragment shares at least 70% identity with a sequence depicted in any one of SEQ ID NOs: 14, 15, 16, 17, or 18.

In another embodiment, the promoter if the expression cassette is selected from the group consisting of (i) a potato granule bound starch synthase gene, (ii) a potato ADP glucose pyrophosphorylase gene, (iii) a potato ubiquitin-7 gene, (iv) a potato patatin gene, (v) a potato flavonoid mono-oxygenase gene. In a further embodiment, the promoter is (i) the promoter of the potato granule bound starch synthase gene shares at least 70% identity with at least part of SEQ ID NO: 12, or (ii) the promoter of a potato ADP glucose pyrophosphorylase gene shares at least 70% identity with at least part of SEQ ID NO: 11, or (iii) the promoter of a potato flavonoid mono-oxygenase gene shares at least 70% identity with at least part of SEQ ID NO: 19.

In one embodiment, a plant of the present invention that is transformed with an expression cassette of the present invention is selected from the group consisting of a potato plant, a wild potato plant, a sweet potato plant, a yam plant, a corn plant, a coffee plant or tree, a cocoa plant or tree, wheat, maize, oats, sorghum, or barley. In another embodiment, the plant is a crop, and the cells of the plant are from a tuber, root, bean, or seed.

In one embodiment, the method for reducing acrylamide levels in a plant further comprises modifying the expression of at least one gene involved in the accumulation of reducing sugars. In one embodiment, the gene involved in the accumulation of reducing sugars is downregulated in expression and selected from the group consisting of (i) a starch-associated R1 gene, and (ii) a starch-associated phosphorylase-L gene. In one embodiment, the step of downregulation comprises introducing into cells of the plant an expression cassette which comprises one or more copies of a fragment of a starch degradation gene operably linked to at least one promoter, wherein expression of the fragment(s) produces at least one of a single-stranded RNA transcript or a double-stranded RNA transcript. In one embodiment, the gene involved in the accumulation of reducing sugars is upregulated in expression and selected from the group consisting of (i) an SnRk1 gene, (ii) a Dofl gene, and (iii) an Mdh gene.

In one embodiment, the method further comprises obtaining a plant product from the plant with reduced asparagine levels, wherein the product has a lower concentration of acrylamide than an equivalent product from a plant of the same species that has not had its asparagine levels reduced. In one embodiment, the heat-processed product is from a potato tuber. In another embodiment, the heat-processed product is a French fry or chip processed from the potato. In another embodiment, the potato, when stored between 4° C. and 12° C. for about one to thirty weeks, contains a glucose level that is less than 50% of the glucose level of an unmodified tuber of the same species stored under the same storage conditions as the potato.

Another aspect of the present invention is an asparagine-reduced potato product that has a level of acrylamide that is at least 10% lower than the level of acrylamide from a non-asparagine-reduced potato product. In one embodiment, the plant product has a level of acrylamide (in parts per billion) selected from the group consisting of (i) 1-20 ppb, (ii) 20-40 ppb, (iii) 40-60 ppb, (iv) 60-80 ppb, and (v) 80-100 ppb of acrylamide. In a further embodiment, the asparagine-reduced potato product is (A) selected from the group consisting of a fried fry, an oven-baked fry, potato chip, hash brown, and tater tots, and (B) the product has a level of acrylamide (in parts per billion) selected from the group consisting of 1-20 ppb, 20-40 ppb, 40-60 ppb, 60-80 ppb, 80-100 ppb, 100-120 ppb, 120-140 ppb, 140-160 ppb, 160-180 ppb, and 180-200 ppb of acrylamide.

Another aspect of the present invention is ground coffee obtained from a coffee plant transformed by the present method which ground coffee has a level of acrylamide that is at least 10% lower than the level of acrylamide of ground coffee from an untransformed plant counterpart, and wherein the acrylamide level is below 100 ppb.

Another aspect of the present invention is roasted coffee from beans with reduced asparagine levels with levels of acrylamide below 100 ppb.

Another aspect of the present invention is a plant product that is wheat bread made from wheat obtained from a wheat plant transformed by the present method, wherein the wheat bread has a level of acrylamide that is at least 10% lower than the level of acrylamide of wheat bread from an untransformed plant counterpart, and wherein the level of acrylamide is below 50 ppb.

Another aspect of the present invention is wheat bread from flour that has reduced asparagine levels with levels of acrylamide below 50 ppb.

Another aspect of the present invention is heat-processed products from potato tubers with reduced asparagine levels with levels of acrylamide below 100 ppb.

The present invention is not limited to any particular crops or plants or products obtained or made from crops or plants. The inventive method is applicable to any plant and vegetable or fruit or product thereof in which it is desirable to modify the expression of certain target genes so as to ultimately reduce the level of acrylamide that is formed in the course of heat-processing that vegetable, fruit, or product. Heat-processing means any form of heating, cooking, microwaving, baking, blanching, grilling, searing, or frying.

### DETAILED DESCRIPTION OF THE PREFERRED EMBODIMENTS

The present invention provides polynucleotide sequences and methods for reducing acrylamide levels.

The present invention uses terms and phrases that are well known to those practicing the art. Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described herein are those well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology (Molecular Cloning, A Laboratory Manual, 3rd. edition, edited by Sambrook & Russel Cold Spring Harbor Laboratory Press, Cold Spring Harbor, N.Y., 2001).

Acrylamide: Acrylamide as a monomer is considered toxic, directly affecting the nervous system. It may be considered a carcinogen. Acrylamide is readily absorbed through intact skin from aqueous solutions. Molecular formula is C3H5NO; structure: CH2=CH—CO—NH2.

Agrobacterium or bacterial transformation: as is well known in the field, Agrobacteria that are used for transforming plant cells are disarmed and virulent derivatives of, usually, Agrobacterium tumefaciens or Agrobacterium rhizogenes. Upon infection of plants, explants, cells, or protoplasts, the Agrobacterium transfers a DNA segment from a plasmid vector to the plant cell nucleus. The vector typically contains a desired polynucleotide that is located between the borders of a T-DNA or P-DNA. However, any bacteria capable of transforming a plant cell may be used, such as, Rhizobium trifolii, Rhizobium leguminosarum, Phyllobacterium myrsinacearum, SinoRhizobium meliloti, and MesoRhizobium loti.

Angiosperm: vascular plants having seeds enclosed in an ovary. Angiosperms are seed plants that produce flowers that bear fruits. Angiosperms are divided into dicotyledonous and monocotyledonous plant.

Aspartate biosynthesis: enzymatically-catalyzed reactions that occur in a plant to produce aspartate.

Aspartate metabolism: enzymatically-catalyzed reactions that occur in a plant to convert aspartate into other compounds.

Antibiotic Resistance: ability of a cell to survive in the presence of an antibiotic. Antibiotic resistance, as used herein, results from the expression of an antibiotic resistance gene in a host cell. A cell may have resistance to any antibiotic. Examples of commonly used antibiotics include kanamycin and hygromycin.

Dicotyledonous plant (dicot): a flowering plant whose embryos have two seed halves or cotyledons, branching leaf veins, and flower parts in multiples of four or five. Examples of dicots include but are not limited to, potato, sugar beet, broccoli, cassaya, sweet potato, pepper, poinsettia, bean, alfalfa, soybean, and avocado.

Endogenous: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Expression cassette: polynucleotide comprising, from 5′ to 3′, (a) a first promoter, (b) a sequence comprising (i) at least one copy of a gene or gene fragment, or (ii) at least one copy of a fragment of the promoter of a gene, and (c) either a terminator or a second promoter that is positioned in the opposite orientation as the first promoter.

Foreign: “foreign,” with respect to a nucleic acid, means that that nucleic acid is derived from non-plant organisms, or derived from a plant that is not the same species as the plant to be transformed or is not derived from a plant that is not interfertile with the plant to be transformed, does not belong to the species of the target plant. According to the present invention, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a polypeptide that is not naturally produced by the transformed plant. A foreign nucleic acid does not have to encode a protein product.

Gene: A gene is a segment of a DNA molecule that contains all the information required for synthesis of a product, polypeptide chain or RNA molecule that includes both coding and non-coding sequences. A gene can also represent multiple sequences, each of which may be expressed independently, and may encode slightly different proteins that display the same functional activity. For instance, the asparagine synthetase 1 and 2 genes can, together, be referred to as a gene.

Genetic element: a “genetic element” is any discreet nucleotide sequence such as, but not limited to, a promoter, gene, terminator, intron, enhancer, spacer, 5′-untranslated region, 3′-untranslated region, or recombinase recognition site.

Genetic modification: stable introduction of DNA into the genome of certain organisms by applying methods in molecular and cell biology.

Gymnosperm: as used herein, refers to a seed plant that bears seed without ovaries. Examples of gymnosperms include conifers, cycads, ginkgos, and ephedras.

Introduction: as used herein, refers to the insertion of a nucleic acid sequence into a cell, by methods including infection, transfection, transformation or transduction.

Monocotyledonous plant (monocot): a flowering plant having embryos with one cotyledon or seed leaf, parallel leaf veins, and flower parts in multiples of three. Examples of monocots include, but are not limited to maize, rice, oat, wheat, barley, and sorghum.

Native: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Native DNA: any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. For instance, a native DNA may comprise a point mutation since such point mutations occur naturally. It is also possible to link two different native DNAs by employing restriction sites because such sites are ubiquitous in plant genomes.

Native Nucleic Acid Construct: a polynucleotide comprising at least one native DNA.

Operably linked: combining two or more molecules in such a fashion that in combination they function properly in a plant cell. For instance, a promoter is operably linked to a structural gene when the promoter controls transcription of the structural gene.

Overexpression: expression of a gene to levels that are higher than those in plants that are not transgenic.

P-DNA: a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterum in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. See Rommens et al. in WO2003/069980, US-2003-0221213, US-2004-0107455, and WO2005/004585, which are all incorporated herein by reference.

Phenotype: phenotype is a distinguishing feature or characteristic of a plant, which may be altered according to the present invention by integrating one or more “desired polynucleotides” and/or screenable/selectable markers into the genome of at least one plant cell of a transformed plant. The “desired polynucleotide(s)” and/or markers may confer a change in the phenotype of a transformed plant, by modifying any one of a number of genetic, molecular, biochemical, physiological, morphological, or agronomic characteristics or properties of the transformed plant cell or plant as a whole. Thus, expression of one or more, stably integrated desired polynucleotide(s) in a plant genome that yields the phenotype of reduced acrylamide concentrations in plant tissues.

Plant tissue: a “plant” or “crop” is any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants and crops such as wheat, maize, rice, barley, oat, sugar beet, coffee, potato, tomato, alfalfa, cassaya, sweet potato, and soybean. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are potato, maize, coffee, and wheat.

Plant transformation and cell culture: broadly refers to the process by which plant cells are genetically modified and transferred to an appropriate plant culture medium for maintenance, further growth, and/or further development. Such methods are well known to the skilled artisan.

Processing: the process of producing a food from (1) the seed of, for instance, wheat, corn, coffee plant, or cocoa tree, (2) the tuber of, for instance, potato, or (3) the root of, for instance, sweet potato and yam, comprising heating the foodstuff in any manner, such as by cooking, baking, frying, grilling, microwaving, searing, boiling, or blanching. The food product may be heated to at least about 100° C., at least about 110° C., at least about 120° C., at least about 130° C., at least about 140° C., at least about 150° C., at least about 160° C., at least about 170° C., at least about 180° C., at least about 190° C., at least about 200° C., at least about 300° C., at least about 400° C., or at least about 500° C., or above, or any integer of degree Celsius in between. Examples of processed foods include bread, breakfast cereal, pies, cakes, toast, biscuits, cookies, pizza, pretzels, tortilla, French fries, oven-baked fries, potato chips, hash browns, roasted coffee, and cocoa.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA or P-DNA construct, which also is inherited by the progeny plant from its parent. The term “progeny” as used herein, also may be considered to be the offspring or descendants of a group of plants.

Promoter: promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. As stated earlier, the RNA generated may code for a protein or polypeptide or may code for an RNA interfering, or antisense molecule.

A promoter is a nucleic acid sequence that enables a gene with which it is associated to be transcribed. In prokaryotes, a promoter typically consists of two short sequences at −10 and −35 position upstream of the gene, that is, prior to the gene in the direction of transcription. The sequence at the −10 position is called the Pribnow box and usually consists of the six nucleotides TATAAT. The Pribnow box is essential to start transcription in prokaryotes. The other sequence at −35 usually consists of the six nucleotides TTGACA, the presence of which facilitates the rate of transcription.

Eukaryotic promoters are more diverse and therefore more difficult to characterize, yet there are certain fundamental characteristics. For instance, eukaryotic promoters typically lie upstream of the gene to which they are most immediately associated. Promoters can have regulatory elements located several kilobases away from their transcriptional start site, although certain tertiary structural formations by the transcriptional complex can cause DNA to fold, which brings those regulatory elements closer to the actual site of transcription. Many eukaryotic promoters contain a “TATA box” sequence, typically denoted by the nucleotide sequence, TATAAA. This element binds a TATA binding protein, which aids formation of the RNA polymerase transcriptional complex. The TATA box typically lies within 50 bases of the transcriptional start site.

Eukaryotic promoters also are characterized by the presence of certain regulatory sequences that bind transcription factors involved in the formation of the transcriptional complex. An example is the E-box denoted by the sequence CACGTG, which binds transcription factors in the basic-helix-loop-helix family. There also are regions that are high in GC nucleotide content.

Hence, according to the present invention, a partial sequence, or a specific promoter “fragment” of a promoter, say for instance of the asparagine synthetase gene, that may be used in the design of a desired polynucleotide of the present invention may or may not comprise one or more of these elements or none of these elements. In one embodiment, a promoter fragment sequence of the present invention is not functional and does not contain a TATA box.

Another characteristic of the construct of the present invention is that it promotes convergent transcription of one or more copies of polynucleotide that is or are not directly operably linked to a terminator, via two opposing promoters. Due to the absence of a termination signal, the length of the pool of RNA molecules that is transcribed from the first and second promoters may be of various lengths.

Occasionally, for instance, the transcriptional machinery may continue to transcribe past the last nucleotide that signifies the “end” of the desired polynucleotide sequence. Accordingly, in this particular arrangement, transcription termination may occur either through the weak and unintended action of downstream sequences that, for instance, promote hairpin formation or through the action of unintended transcriptional terminators located in plant DNA flanking the transfer DNA integration site.

The desired polynucleotide may be linked in two different orientations to the promoter. In one orientation, e.g., “sense”, at least the 5′-part of the resultant RNA transcript will share sequence identity with at least part of at least one target transcript. In the other orientation designated as “antisense”, at least the 5′-part of the predicted transcript will be identical or homologous to at least part of the inverse complement of at least one target transcript.

A plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as xylem, leaves, roots, or seeds. Such promoters are referred to as tissue-preferred promoters. Promoters which initiate transcription only in certain tissues are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Polynucleotide is a nucleotide sequence, comprising a gene coding sequence or a fragment thereof, such as comprising, for instance, at least 15 consecutive nucleotides, at least 30 consecutive nucleotides, or at least 50 consecutive nucleotides, a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, an RNA transcript (such as an mRNA) or a processed nucleotide sequence (such as a cDNA). The polynucleotide may comprise a sequence in either sense or antisense orientations.

An isolated polynucleotide is a polynucleotide sequence that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

Seed: a “seed” may be regarded as a ripened plant ovule containing an embryo, and a propagative part of a plant, as a tuber or spore. Seed may be incubated prior to Agrobacterium-mediated transformation, in the dark, for instance, to facilitate germination. Seed also may be sterilized prior to incubation, such as by brief treatment with bleach. The resultant seedling can then be exposed to a desired strain of Agrobacterium.

Selectable/screenable marker: a gene that, if expressed in plants or plant tissues, makes it possible to distinguish them from other plants or plant tissues that do not express that gene. Screening procedures may require assays for expression of proteins encoded by the screenable marker gene. Examples of selectable markers include the neomycin phosphotransferase (NptII) gene encoding kanamycin and geneticin resistance, the hygromycin phosphotransferase (HptII) gene encoding resistance to hygromycin, or other similar genes known in the art.

Sensory characteristics: panels of professionally trained individuals can rate food products for sensory characteristics such as appearance, flavor, aroma, and texture. Thus, the present invention contemplates improving the sensory characteristics of a plant product obtained from a plant that has been modified according to the present invention to manipulate its asparagine biosynthesis and metabolism pathways.

Sequence identity: as used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS 1N MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

Silencing: The unidirectional and unperturbed transcription of either genes or gene fragments from promoter to terminator can trigger post-transcriptional silencing of target genes. Initial expression cassettes for post-transcriptional gene silencing in plants comprised a single gene fragment positioned in either the antisense (McCormick et al., U.S. Pat. No. 6,617,496; Shewmaker et al., U.S. Pat. No. 5,107,065) or sense (van der Krol et al., Plant Cell 2:291-299, 1990) orientation between regulatory sequences for transcript initiation and termination. In Arabidopsis, recognition of the resulting transcripts by RNA-dependent RNA polymerase leads to the production of double-stranded (ds) RNA. Cleavage of this dsRNA by Dicer-like (Dcl) proteins such as Dcl4 yields 21-nucleotide (nt) small interfering RNAs (siRNAs). These siRNAs complex with proteins including members of the Argonaute (Ago) family to produce RNA-induced silencing complexes (RISCs). The RISCs then target homologous RNAs for endonucleolytic cleavage.

More effective silencing constructs contain both a sense and antisense component, producing RNA molecules that fold back into hairpin structures. The high dsRNA levels produced by expression of inverted repeat transgenes were hypothesized to promote the activity of multiple Dcls. Analyses of combinatorial Dcl knockouts in Arabidopsis supported this idea, and also identified Dcl4 as one of the proteins involved in RNA cleavage.

One component of conventional sense, antisense, and double-strand (ds) RNA-based gene silencing constructs is the transcriptional terminator. WO 2006/036739 shows that this regulatory element becomes obsolete when gene fragments are positioned between two oppositely oriented and functionally active promoters. The resulting convergent transcription triggers gene silencing that is at least as effective as unidirectional ‘promoter-to-terminator’ transcription. In addition to short variably-sized and non-polyadenylated RNAs, terminator-free cassette produced rare longer transcripts that reach into the flanking promoter. Replacement of gene fragments by promoter-derived sequences further increased the extent of gene silencing.

In an embodiment of the present invention, the desired polynucleotide comprises a partial sequence of a target gene promoter or a partial sequence that shares sequence identity with a portion of a target gene promoter. Hence, a desired polynucleotide of the present invention contains a specific fragment of a particular target gene promoter of interest.

The desired polynucleotide may be operably linked to one or more functional promoters. Various constructs contemplated by the present invention include, but are not limited to (1) a construct where the desired polynucleotide comprises one or more promoter fragment sequences and is operably linked at both ends to functional ‘driver’ promoters. Those two functional promoters are arranged in a convergent orientation so that each strand of the desired polynucleotide is transcribed; (2) a construct where the desired polynucleotide is operably linked to one functional promoter at either its 5′-end or its 3′-end, and the desired polynucleotide is also operably linked at its non-promoter end by a functional terminator sequence; (3) a construct where the desired polynucleotide is operably linked to one functional promoter at either its 5′-end or its 3′-end, but where the desired polynucleotide is not operably linked to a terminator; or (4) a cassette, where the desired polynucleotide comprises one or more promoter fragment sequences but is not operably linked to any functional promoters or terminators.

In another embodiment of the present invention, the desired polynucleotide comprises a partial sequence of any part of a target gene sequence, such as a partial sequence of the coding or structural sequence of the target gene, or a sequence from an exon or intron of the target gene, or a sequence from the 5′- or 3′-untranslated region of the target gene. Accordingly, a desired polynucleotide, which comprises such a partial sequence, may be operably linked to one or more functional promoters. Various constructs contemplated by the present invention include, but are not limited to (1) a construct where the desired polynucleotide comprises one or more target gene sequences and is operably linked at both ends to functional ‘driver’ promoters. Those two functional promoters can be arranged in a convergent orientation so that each strand of the desired polynucleotide is transcribed; (2) a construct where the desired polynucleotide is operably linked to one functional promoter at either its 5′-end or its 3′-end, and the desired polynucleotide is also operably linked at its non-promoter end to a functional terminator sequence; (3) a construct where the desired polynucleotide is operably linked to one functional promoter at either its 5′-end or its 3′-end, but where the desired polynucleotide is not operably linked to a terminator; or (4) a cassette, where the desired polynucleotide comprises a target gene sequence but is not operably linked to any functional promoters or terminators.

In any of the constructs of the present invention, regardless of which target gene sequence is incorporated into the desired polynucleotide, the desired polynucleotide can be operably linked to functional promoters but not to any functional terminator sequences.

A construct of the present invention may comprise two or more ‘driver’ promoters which flank one or more desired polynucleotides or which flank copies of a desired polynucleotide, such that both strands of the desired polynucleotide are transcribed. That is, one promoter may be oriented to initiate transcription of the 5′-end of a desired polynucleotide, while a second promoter may be operably oriented to initiate transcription from the 3′-end of the same desired polynucleotide. The oppositely-oriented promoters may flank multiple copies of the desired polynucleotide. Hence, the “copy number” may vary so that a construct may comprise 2, 3, 4, 5, 6, 7, 8, 9, 10, 15, 20, 30, 40, 50, 60, 70, 80, 90, or 100, or more than 100 copies, or any integer in-between, of a desired polynucleotide, which may be flanked by the ‘driver’ promoters that are oriented to induce convergent transcription.

If neither cassette comprises a terminator sequence, then such a construct, by virtue of the convergent transcription arrangement, may produce RNA transcripts that are of different lengths.

In this situation, therefore, there may exist subpopulations of partially or fully transcribed RNA transcripts that comprise partial or full-length sequences of the transcribed desired polynucleotide from the respective cassette. Alternatively, in the absence of a functional terminator, the transcription machinery may proceed past the end of a desired polynucleotide to produce a transcript that is longer than the length of the desired polynucleotide.

In a construct that comprises two copies of a desired polynucleotide, therefore, where one of the polynucleotides may or may not be oriented in the inverse complementary direction to the other, and where the polynucleotides are operably linked to promoters to induce convergent transcription, and there is no functional terminator in the construct, the transcription machinery that initiates from one desired polynucleotide may proceed to transcribe the other copy of the desired polynucleotide and vice versa. The multiple copies of the desired polynucleotide may be oriented in various permutations: in the case where two copies of the desired polynucleotide are present in the construct, the copies may, for example, both be oriented in same direction, in the reverse orientation to each other, or in the inverse complement orientation to each other, for example.

In an arrangement where one of the desired polynucleotides is oriented in the inverse complementary orientation to the other polynucleotide, an RNA transcript may be produced that comprises not only the “sense” sequence of the first polynucleotide but also the “antisense” sequence from the second polynucleotide. If the first and second polynucleotides comprise the same or substantially the same DNA sequences, then the single RNA transcript may comprise two regions that are complementary to one another and which may, therefore, anneal. Hence, the single RNA transcript that is so transcribed, may form a partial or full hairpin duplex structure.

On the other hand, if two copies of such a long transcript were produced, one from each promoter, then there will exist two RNA molecules, each of which would share regions of sequence complementarity with the other. Hence, the “sense” region of the first RNA transcript may anneal to the “antisense” region of the second RNA transcript and vice versa. In this arrangement, therefore, another RNA duplex may be formed which will consist of two separate RNA transcripts, as opposed to a hairpin duplex that forms from a single self-complementary RNA transcript.

Alternatively, two copies of the desired polynucleotide may be oriented in the same direction so that, in the case of transcription read-through, the long RNA transcript that is produced from one promoter may comprise, for instance, the sense sequence of the first copy of the desired polynucleotide and also the sense sequence of the second copy of the desired polynucleotide. The RNA transcript that is produced from the other convergently-oriented promoter, therefore, may comprise the antisense sequence of the second copy of the desired polynucleotide and also the antisense sequence of the first polynucleotide. Accordingly, it is likely that neither RNA transcript would contain regions of exact complementarity and, therefore, neither RNA transcript is likely to fold on itself to produce a hairpin structure. On the other hand the two individual RNA transcripts could hybridize and anneal to one another to form an RNA duplex.

Hence, in one aspect, the present invention provides a construct that lacks a terminator or lacks a terminator that is preceded by self-splicing ribozyme encoding DNA region, but which comprises a first promoter that is operably linked to the desired polynucleotide.

Variety: commercial and genetically stable genotype. The current invention targets a variety of a starch-rich food crop that is used to produce heat processed foods such as French fries, potato chips, bread, biscuits, and coffee. A potato variety that is transformed to reduce asparagine levels can be selected from the group consisting of Ranger Russet, Russet Burbank, Atlantic, Shepody, Alturas, Gem Russet, Bintje, Innovator, and proprietary varieties including those developed by companies such as Frito-Lay.

Tissue: any part of a plant that is used to produce a food. A tissue can be a tuber of a potato, a root of a sweet potato, or a seed of a maize plant.

Transcriptional terminators: The expression DNA constructs of the present invention may have a transcriptional termination region at the opposite end from the transcription initiation regulatory region. The transcriptional termination region may be selected, for stability of the mRNA to enhance expression and/or for the addition of polyadenylation tails added to the gene transcription product. Translation of a nascent polypeptide undergoes termination when any of the three chain-termination codons enters the A site on the ribosome. Translation termination codons are UAA, UAG, and UGA.

In the instant invention, transcription terminators are derived from either a gene or, more preferably, from a sequence that does not represent a gene but intergenic DNA. For example, the terminator sequence from the potato ubiquitin gene may be used and is depicted in SEQ ID NO: 13.

Transfer DNA (T-DNA): a transfer DNA is a DNA segment delineated by either T-DNA borders or P-DNA borders to create a T-DNA or P-DNA, respectively. A T-DNA is a genetic element that is well-known as an element capable of integrating a nucleotide sequence contained within its borders into another genome. In this respect, a T-DNA is flanked, typically, by two “border” sequences. A desired polynucleotide of the present invention and a selectable marker may be positioned between the left border-like sequence and the right border-like sequence of a T-DNA. The desired polynucleotide and selectable marker contained within the T-DNA may be operably linked to a variety of different, plant-specific (i.e., native), or foreign nucleic acids, like promoter and terminator regulatory elements that facilitate its expression, i.e., transcription and/or translation of the DNA sequence encoded by the desired polynucleotide or selectable marker.

Transformation of plant cells: A process by which a nucleic acid is stably inserted into the genome of a plant cell. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols such as ‘refined transformation’ or ‘precise breeding’, viral infection, whiskers, electroporation, microinjection, polyethylene glycol-treatment, heat shock, lipofection and particle bombardment.

Transgenic plant: a transgenic plant of the present invention is one that comprises at least one cell genome in which an exogenous nucleic acid has been stably integrated. According to the present invention, a transgenic plant is a plant that comprises only one genetically modified cell and cell genome, or is a plant that comprises some genetically modified cells, or is a plant in which all of the cells are genetically modified. A transgenic plant of the present invention may be one that comprises expression of the desired polynucleotide, i.e., the exogenous nucleic acid, in only certain parts of the plant. Thus, a transgenic plant may contain only genetically modified cells in certain parts of its structure.

Variant: a “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” may also refer to a “shuffled gene” such as those described in Maxygen-assigned patents.

It is understood that the present invention is not limited to the particular methodology, protocols, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a gene” is a reference to one or more genes and includes equivalents thereof known to those skilled in the art and so forth. Indeed, one skilled in the art can use the methods described herein to express any native gene (known presently or subsequently) in plant host systems.

Any or all of the elements and DNA sequences that are described herein may be endogenous to one or more plant genomes. Accordingly, in one particular embodiment of the present invention, all of the elements and DNA sequences, which are selected for the ultimate transfer cassette are endogenous to, or native to, the genome of the plant that is to be transformed. For instance, all of the sequences may come from a potato genome. Alternatively, one or more of the elements or DNA sequences may be endogenous to a plant genome that is not the same as the species of the plant to be transformed, but which function in any event in the host plant cell. Such plants include potato, tomato, and alfalfa plants. The present invention also encompasses use of one or more genetic elements from a plant that is interfertile with the plant that is to be transformed.

In this regard, a “plant” of the present invention includes, but is not limited to potato, tomato, avocado, alfalfa, sugarbeet, cassaya, sweet potato, soybean, pea, bean, maize, wheat, rice, barley, and sorghum. Thus, a plant may be a monocot or a dicot. “Plant” and “plant material,” also encompasses plant cells, seed, plant progeny, propagule whether generated sexually or asexually, and descendents of any of these, such as cuttings or seed. “Plant material” may refer to plant cells, cell suspension cultures, callus, embryos, meristematic regions, callus tissue, leaves, roots, shoots, gametophytes, sporophytes, pollen, seeds, germinating seedlings, and microspores. Plants may be at various stages of maturity and may be grown in liquid or solid culture, or in soil or suitable media in pots, greenhouses or fields. Expression of an introduced leader, trailer or gene sequences in plants may be transient or permanent.

In this respect, a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterum in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. See Rommens et al. in WO2003/069980, US-2003-0221213, US-2004-0107455, and WO2005/004585, which are all incorporated herein by reference.

Thus, a P-DNA border sequence is different by 1, 2, 3, 4, 5, 6, 7, 8, 9, 10, 11, 12, 13, 14, 15, 16, 17, 18, 19, 20, or more nucleotides from a known T-DNA border sequence from an Agrobacterium species, such as Agrobacterium tumefaciens or Agrobacterium rhizogenes.

A P-DNA border sequence is not greater than 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51% or 50% similar in nucleotide sequence to an Agrobacterium T-DNA border sequence.

Methods were developed to identify and isolate transfer DNAs from plants, particularly potato and wheat, and made use of the border motif consensus described in US-2004-0107455, which is incorporated herein by reference.

In this respect, a plant-derived DNA of the present invention, such as any of the sequences, cleavage sites, regions, or elements disclosed herein is functional if it promotes the transfer and integration of a polynucleotide to which it is linked into another nucleic acid molecule, such as into a plant chromosome, at a transformation frequency of about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 15%, or about 5% or at least about 1%.

Any of such transformation-related sequences and elements can be modified or mutated to change transformation efficiency. Other polynucleotide sequences may be added to a transformation sequence of the present invention. For instance, it may be modified to possess 5′- and 3′-multiple cloning sites, or additional restriction sites. The sequence of a cleavage site as disclosed herein, for example, may be modified to increase the likelihood that backbone DNA from the accompanying vector is not integrated into a plant genome.

Any desired polynucleotide may be inserted between any cleavage or border sequences described herein. For example, a desired polynucleotide may be a wild-type or modified gene that is native to a plant species, or it may be a gene from a non-plant genome. For instance, when transforming a potato plant, an expression cassette can be made that comprises a potato-specific promoter that is operably linked to a desired potato gene or fragment thereof and a potato-specific terminator. The expression cassette may contain additional potato genetic elements such as a signal peptide sequence fused in frame to the 5′-end of the gene, and a potato transcriptional enhancer. The present invention is not limited to such an arrangement and a transformation cassette may be constructed such that the desired polynucleotide, while operably linked to a promoter, is not operably linked to a terminator sequence.

When a transformation-related sequence or element, such as those described herein, are identified and isolated from a plant, and if that sequence or element is subsequently used to transform a plant of the same species, that sequence or element can be described as “native” to the plant genome.

Thus, a “native” genetic element refers to a nucleic acid that naturally exists in, originates from, or belongs to the genome of a plant that is to be transformed. In the same vein, the term “endogenous” also can be used to identify a particular nucleic acid, e.g., DNA or RNA, or a protein as “native” to a plant. Endogenous means an element that originates within the organism. Thus, any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. In this respect, a “native” nucleic acid may also be isolated from a plant or sexually compatible species thereof and modified or mutated so that the resultant variant is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in nucleotide sequence to the unmodified, native nucleic acid isolated from a plant. A native nucleic acid variant may also be less than about 60%, less than about 55%, or less than about 50% similar in nucleotide sequence.

A “native” nucleic acid isolated from a plant may also encode a variant of the naturally occurring protein product transcribed and translated from that nucleic acid. Thus, a native nucleic acid may encode a protein that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in amino acid sequence to the unmodified, native protein expressed in the plant from which the nucleic acid was isolated.

### Promoters

The polynucleotides of the present invention can be used for specifically directing the expression of polypeptides or proteins in the tissues of plants. The nucleic acids of the present invention can also be used for specifically directing the expression of antisense RNA, or RNA involved in RNA interference (RNAi) such as small interfering RNA (siRNA), in the tissues of plants, which can be useful for inhibiting or completely blocking the expression of targeted genes. As used herein, “coding product” is intended to mean the ultimate product of the nucleic acid that is operably linked to the promoters. For example, a protein or polypeptide is a coding product, as well as antisense RNA or siRNA which is the ultimate product of the nucleic acid coding for the antisense RNA. The coding product may also be non-translated mRNA. The terms polypeptide and protein are used interchangeably herein. As used herein, promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. The RNA may code for a protein or polypeptide or may code for an RNA interfering, or antisense molecule. As used herein, “operably linked” is meant to refer to the chemical fusion, ligation, or synthesis of DNA such that a promoter-nucleic acid sequence combination is formed in a proper orientation for the nucleic acid sequence to be transcribed into an RNA segment. The promoters of the current invention may also contain some or all of the 5′ untranslated region (5′ UTR) of the resulting mRNA transcript. On the other hand, the promoters of the current invention do not necessarily need to possess any of the 5′ UTR.

A promoter, as used herein, may also include regulatory elements. Conversely, a regulatory element may also be separate from a promoter. Regulatory elements confer a number of important characteristics upon a promoter region. Some elements bind transcription factors that enhance the rate of transcription of the operably linked nucleic acid. Other elements bind repressors that inhibit transcription activity. The effect of transcription factors on promoter activity may determine whether the promoter activity is high or low, i.e. whether the promoter is “strong” or “weak.”

In another embodiment, a constitutive promoter may be used for expressing the inventive polynucleotide sequences.

In another embodiment, a variety of inducible plant gene promoters can be used for expressing the inventive polynucleotide sequences. Inducible promoters regulate gene expression in response to environmental, hormonal, or chemical signals. Examples of hormone inducible promoters include auxin-inducible promoters (Baumann et al. Plant Cell 11:323-334 (1999)), cytokinin-inducible promoter (Guevara-Garcia Plant Mol. Biol. 38:743-753 (1998)), and gibberellin-responsive promoters (Shi et al. Plant Mol. Biol. 38:1053-1060 (1998)). Additionally, promoters responsive to heat, light, wounding, pathogen resistance, and chemicals such as methyl jasmonate or salicylic acid, may be used for expressing the inventive polynucleotide sequences.

In one embodiment, the promoter is a granule bound starch synthase promoter, a potato ADP-glucose pyrophosphorylase gene promoter, or a flavonoid 3′-monooxygenase gene promoter. In another embodiment, the promoter is a seed-specific promoter.

The present invention also encompasses polynucleotides that differ from the disclosed sequences but that, as a consequence of the degeneracy of the genetic code, encode a polypeptide which is the same as that encoded by a polynucleotide of the present invention. Thus, polynucleotides comprising sequences that differ from the polynucleotide sequences recited in SEQ ID NOs: 1-10, 14-18, and 20-39 or complements, reverse sequences, or reverse complements thereof, as a result of conservative substitutions are contemplated by and encompassed within the present invention. Additionally, polynucleotides comprising sequences that differ from the polynucleotide sequences recited in SEQ ID NOs: 1-10, 14-18, and 20-39 or complements, reverse complements or reverse sequences thereof, as a result of deletions and/or insertions totaling less than 10% of the total sequence length are also contemplated by and encompassed within the present invention.

In addition to having a specified percentage identity to an inventive polynucleotide sequence, variant polynucleotides preferably have additional structure and/or functional features in common with the inventive polynucleotide. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they have domains in common.

### Source of Elements and DNA Sequences

Any or all of the elements and DNA sequences that are described herein may be endogenous to one or more plant genomes. Accordingly, in one particular embodiment of the present invention, all of the elements and DNA sequences, which are selected for the ultimate transfer cassette are endogenous to, or native to, the genome of the plant that is to be transformed. For instance, all of the sequences may come from a potato genome. Alternatively, one or more of the elements or DNA sequences may be endogenous to a plant genome that is not the same as the species of the plant to be transformed, but which function in any event in the host plant cell. Such plants include potato, tomato, and alfalfa plants. The present invention also encompasses use of one or more genetic elements from a plant that is interfertile with the plant that is to be transformed.

In this regard, a “plant” of the present invention includes, but is not limited to potato, tomato, alfalfa, sugarbeet, cassava, sweet potato, soybean, pea, bean, maize, wheat, rice, barley, and sorghum. “Plant” and “plant material,” also encompasses plant cells, seed, plant progeny, propagule whether generated sexually or asexually, and descendents of any of these, such as cuttings or seed. “Plant material” may refer to plant cells, cell suspension cultures, callus, embryos, meristematic regions, callus tissue, leaves, roots, shoots, gametophytes, sporophytes, pollen, seeds, germinating seedlings, and microspores. Plants may be at various stages of maturity and may be grown in liquid or solid culture, or in soil or suitable media in pots, greenhouses or fields. Expression of an introduced leader, trailer or gene sequences in plants may be transient or permanent.

### Nucleic Acid Constructs

The present invention provides constructs comprising the isolated nucleic acid molecules and polypeptide sequences of the present invention. In one embodiment, the DNA constructs of the present invention are Ti-plasmids derived from A. tumefaciens.

In developing the nucleic acid constructs of this invention, the various components of the construct or fragments thereof will normally be inserted into a convenient cloning vector, e.g., a plasmid that is capable of replication in a bacterial host, e.g., E. coli. Numerous vectors exist that have been described in the literature, many of which are commercially available. After each cloning, the cloning vector with the desired insert may be isolated and subjected to further manipulation, such as restriction digestion, insertion of new fragments or nucleotides, ligation, deletion, mutation, resection, etc. to tailor the components of the desired sequence. Once the construct has been completed, it may then be transferred to an appropriate vector for further manipulation in accordance with the manner of transformation of the host cell.

A recombinant DNA molecule of the invention may typically include a selectable marker so that transformed cells can be easily identified and selected from non-transformed cells. Examples of such markers include, but are not limited to, a neomycin phosphotransferase (nptII) gene (Potrykus et al., Mol. Gen. Genet. 199:183-188 (1985)), which confers kanamycin resistance. Cells expressing the nptII gene can be selected using an appropriate antibiotic such as kanamycin or G418. Other commonly used selectable markers include the bar gene, which confers bialaphos resistance; a mutant EPSP synthase gene (Hinchee et al., Bio/Technology 6:915-922 (1988)), which confers glyphosate resistance; and a mutant acetolactate synthase gene (ALS), which confers imidazolinone or sulphonylurea resistance (European Patent Application 154,204, 1985).

Additionally, vectors may include an origin of replication (replicons) for a particular host cell. Various prokaryotic replicons are known to those skilled in the art, and function to direct autonomous replication and maintenance of a recombinant molecule in a prokaryotic host cell.

The invention also provides host cells which comprise the DNA constructs of the current invention. As used herein, a host cell refers to the cell in which the coding product is ultimately expressed. Accordingly, a host cell can be an individual cell, a cell culture or cells as part of an organism.

Accordingly, the present invention also provides plants or plant cells, comprising the DNA constructs of the current invention. Preferably the plants are angiosperms or gymnosperms. The expression construct of the present invention may be used to transform a variety of plants, both monocotyledonous (e.g. wheat, turf grass, maize, rice, oat, wheat, barley, sorghum, orchid, iris, lily, onion, banana, sugarcane, and palm), dicotyledonous (e.g., Arabidopsis, potato, tobacco, tomato, avocado, pepper, sugarbeet, broccoli, cassaya, sweet potato, cotton, poinsettia, legumes, alfalfa, soybean, pea, bean, cucumber, grape, brassica, carrot, strawberry, lettuce, oak, maple, walnut, rose, mint, squash, daisy, and cactus, oaks, eucalyptus, maple), and Gymnosperms (e.g., Scots pine; see Aronen, Finnish Forest Res. Papers, Vol. 595, 1996), white spruce (Ellis et al., Biotechnology 11:84-89, 1993), and larch (Huang et al., In Vitro Cell 27:201-207, 1991).

### Plant Transformation and Regeneration

The present polynucleotides and polypeptides may be introduced into a host plant cell by standard procedures known in the art for introducing recombinant sequences into a target host cell. Such procedures include, but are not limited to, transfection, infection, transformation, natural uptake, electroporation, biolistics and Agrobacterium. Methods for introducing foreign genes into plants are known in the art and can be used to insert a construct of the invention into a plant host, including, biological and physical plant transformation protocols. See, for example, Miki et al., 1993, “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch et al., Science 227:1229-31, 1985), electroporation, micro-injection, and biolistic bombardment. Preferred transformation methods include precise breeding (see: United States patent applications 2003/0221213 A1, 2004/0107455 A1, and 2005/0229267 A1) and refined transformation (see United States patent application 2005/0034188 A1).

Accordingly, the present invention also provides plants or plant cells, comprising the polynucleotides or polypeptides of the current invention. In one embodiment, the plants are angiosperms or gymnosperms. Beyond the ordinary meaning of plant, the term “plants” is also intended to mean the fruit, seeds, flower, strobilus etc. of the plant. The plant of the current invention may be a direct transfectant, meaning that the vector was introduced directly into the plant, such as through Agrobacterium, or the plant may be the progeny of a transfected plant. The progeny may also be obtained by asexual reproduction of a transfected plant. The second or subsequent generation plant may or may not be produced by sexual reproduction, i.e., fertilization. Furthermore, the plant can be a gametophyte (haploid stage) or a sporophyte (diploid stage).

In this regard, the present invention contemplates transforming a plant with one or more transformation elements that genetically originate from a plant. The present invention encompasses an “all-native” approach to transformation, whereby only transformation elements that are native to plants are ultimately integrated into a desired plant via transformation. In this respect, the present invention encompasses transforming a particular plant species with only genetic transformation elements that are native to that plant species. The native approach may also mean that a particular transformation element is isolated from the same plant that is to be transformed, the same plant species, or from a plant that is sexually interfertile with the plant to be transformed.

On the other hand, the plant that is to be transformed, may be transformed with a transformation cassette that contains one or more genetic elements and sequences that originate from a plant of a different species. It may be desirable to use, for instance, a cleavage site, that is native to a potato genome in a transformation cassette or plasmid for transforming a tomato or pepper plant.

The present invention is not limited, however, to native or all-native approach. A transformation cassette or plasmid of the present invention can also comprise sequences and elements from other organisms, such as from a bacterial species.

The following examples are set forth as representative of specific and preferred embodiments of the present invention. These examples are not to be construed as limiting the scope of the invention in any manner. It should be understood that many variations and modifications can be made while remaining within the spirit and scope of the invention.

### EXAMPLES

### Example 1

### Tuber-Specific Aspartate Aminotransferase Gene Silencing

This example demonstrates that the down-regulated expression of a gene involved in aspartate biosynthesis in the starchy tissues of a crop lowers the amount of aspartate and asparagine in these starchy tissues and, consequently, lowers the amount of acrylamide in a food obtained from heating these starchy tissues.

Aspartate aminotransferase (Aat; EC 2.6.1.1) is ubiquitous in prokaryotes and eukaryotes and has a crucial role in both C and N metabolism. The enzyme catalyzes the reversible transfer of the amino group from glutamate to oxaloacetate, yielding 2-oxoglutarate and aspartate. In plants, Aat is involved in many metabolic pathways: the synthesis of aspartate and asparagine for nitrogen transport to the shoot, for protein and amino acid synthesis, and organic acid synthesis to support plant respiration, and the assimilation of ammonia in the glutamine synthetase/glutamate synthase (GS/GOGAT) cycle. Aat has also been proposed to play a role in malate-aspartate hydrogen shuttles that distribute reducing equivalents across membranes of chloroplasts, mitochondria, and peroxisomes.

Potato contains a number of different Aat genes, and it may therefore be useful to target one or two or more than two of these Aat gene isoforms in tuber tissues to lower the accumulation of asparagine. One efficacious silencing construct contains an inverted repeat inserted between two promoters that are functional in tuber tissues whereby each of the repeats contains 181 to 183-bp fragments of five different Aat genes. These fragments are derived from various Aat genes shown in SEQ ID NOs: 1-5. The sequences of the fragments themselves are shown as SEQ ID NOs: 6-10. A spacer can be inserted between the repeated sequences to facilitate cloning. The regulatory elements of the silencing construct can comprise two convergently-oriented promoters that are predominantly active in tubers such as the promoter of the ADP-glucose pyrophosphorylase (Agp) gene (SEQ ID NO: 11) and the promoter of the granule-bound starch synthase (Gbss) gene (SEQ ID NO: 12).

Introduction of the silencing cassette into plants results in the production of variably-sozed transcripts through collisional convergent transcription. These unpolyadenylated RNAs will trigger gene silencing in a similar way as described (Yan et al., New construct approaches for efficient gene silencing in plants. Plant Physiol 141:1508-1518). Alternatively, it is possible to create silencing cassettes for unidirectional transcription that comprise a promoter and downstream terminator from a gene such as the potato ubiquitin-3 gene. The sequence for this terminator is shown in SEQ ID NO: 13.

A binary vector carrying the silencing cassette for the Aat genes can be introduced into Agrobacterium LBA4404 as follows. Competent LB4404 cells (50 μL) are incubated for 5 min on ice in the presence of 1 μg of vector DNA, frozen for about 15 s in liquid nitrogen, and incubated at 37° C. for 5 min. After adding 1 mL of liquid broth, the treated cells are grown for 3 h at 28° C. and plated on liquid broth/agar containing streptomycin (100 mg/L) and kanamycin (100 mg/L). The vector DNAs are then isolated from overnight cultures of individual LBA4404 colonies and examined by restriction analysis to confirm the presence of intact plasmid DNA.

Ten-fold dilutions of overnight-grown Agrobacterium cultures can be grown for 5-6 hours, precipitated for 15 minutes at 2,800 RPM, washed with MS liquid medium (Phytotechnology) supplemented with sucrose (3%, pH 5.7), and resuspended in the same medium to 0.2 OD/600 nm. The resuspended cells are mixed and used to infect 0.4-0.6 mm internodal segments of the potato varieties Ranger Russet, Russet Burbank, Atlantic, Shepody.

Infected stems are incubated for two days on co-culture medium ( 1/10 MS salts, 3% sucrose, pH 5.7) containing 6 g/L agar at 22° C. in a Percival growth chamber (16 hrs light) and subsequently transferred to callus induction medium (CIM, MS medium supplemented with 3% sucrose 3, 2.5 mg/L of zeatin riboside, 0.1 mg/L of naphthalene acetic acid, and 6 g/L of agar) containing timentin (150 mg/L) and kanamycin (100 mg/L). After one month of culture on CIM, explants were transferred to shoot induction medium (SIM, MS medium supplemented with 3% sucrose, 2.5 mg/L of zeatin riboside, 0.3 mg/L of giberellic acid GA3, and 6 g/L of agar) containing timentin and kanamycin (150 and 100 mg/L respectively) until shoots arose. Shoots arising at the end of regeneration period were transferred to MS medium with 3% sucrose, 6 g/L of agar and timentin (150 mg/L). Transgenic plants were transferred to soil and placed in a greenhouse.

After three months, tubers are harvested and analyzed for asparagine levels according to the Official Methods of Analysis of AOAC INTERNATIONAL (2002), 17th Edition, AOAC INTERNATIONAL, Gaithersburg, Md., USA Official Method 982.30. This analysis will show that a proportion of transgenic plants produced tubers containing reduced amounts of free asparagine.

Tubers from the low asparagine plants can be cut, blanched, par-fried, and finish-fried to produce French fries. They can also be used to produce potato chips. These French fries or chips are ground to a fine powder in liquid nitrogen that can be shipped on dry ice to Covance laboratories. At Covance, acrylamide levels are determined by performing liquid chromatography/mass spectrometry/mass spectrometry (LC/MS/MS) (United States Food and Drug Administration, Center for Food Safety and Applied Nutrition Office of Plant & Dairy Foods and Beverages, “Detection and Quantitation of Acrylamide in Foods”, 2002). This analysis will show that fries from the low asparagine tubers accumulate less than about a third of the acrylamide that is produced in control fries.

As an alternative to using the Agp and Gbss promoters as regulatory elements, it is also possible to employ two Gbss promoters. Furthermore, Aat gene-derived sequences can be inserted between a promoter and a terminator.

Aat genes can be either identified by searching databases or isolated from plant DNA. Following identification of a plant-derived Aat gene, gene-specific primers are designed for PCR-amplification of the gene. PCR amplification is performed according to methods known in the art and then the PCR amplified Aat gene is cloned into a cloning vector.

One example of Ast genes from a crop other than potato relates to the Aat genes from wheat. Simultaneous silencing of these genes in the wheat grain will make it possible to reduce asparagine levels in flour and, consequently, acrylamide levels in, for instance, bread, biscuits, cookies, or crackers. Similarly, conventional transformation methods can be used to transform crops such as coffee with silencing constructs that down-regulate the expression of Aat genes in beans.

Instead of using Aat gene fragments for production of a silencing construct, it is also possible to employ fragments obtained from the promoters of the Aat genes. Such promoters can be isolated by applying methods such as inverse PCR, and two copies of specific 150-600-basepair promoter fragments can then be inserted as inverted repeat between either a promoter and terminator or two convergently-oriented promoters (see: Rommens et al., World Patent application 2006/036739 A2, which is incorporated herein by reference).

### Example 2

### Tuber-Specific Overexpression of a Lysine Feed-Back Less-Sensitive Form of Aspartate Kinase

This example demonstrates that overexpression of a gene involved in aspartate metabolism in the starchy tissues of a crop lowers the amount of aspartate and asparagine in these starchy tissues and, consequently, limits the accumulation of acrylamide in a food obtained from heating these starchy tissues.

In order to modify the flux through the aspartate family pathway and enhance the accumulation of the essential amino acids free lysine and free methionine while lowering accumulation of free asparagine, plants can be transformed with expression cassettes for the lysine feed-back less-sensitive forms of the aspartate kinase (AK) gene. One example of such a gene delineated by BamHI and EcoRI sites is shown in SEQ ID NO: 14. The AK gene can to be fused to a upstream sequence encoding a chloroplast signal peptide to target the encoded protein to the chloroplast. Such a sequence can be obtained from, for instance, the pea rbcS gene (accession number J01257). A 1,604-base pair BamHI-EcoRI fragment comprising the feedback insensitive AK gene fused to an untranslated leader sequence that enhances expression as well as a signal peptide coding sequence is shown as SEQ ID 15. An alternative 1,604-base pair fragment contains the leader and signal peptide sequence fused to an AK gene that was codon-modified to optimize expression in potato is shown as SEQ ID NO: 16.

As an alternative to the AK gene shown in SEQ ID NO: 14, it is also possible to use either the feedback insensitive AK gene from Streptomyces albulus shown in SEQ ID NO: 17 or a codon-optimized derivative gene (SEQ ID NO: 18).

Other promoters that can be used to drive AK gene expression in potato tubers can be selected from the group consisting of patatin promoters, cold-inducible promoters, and flavonoid-3′-mono-oxygenase (Fmo) promoters. One Fmo promoter is shown in SEQ ID NO: 19. This promoter is active in semi-mature and mature tubers but not in mini-tubers.

For any AK gene, there may be other sequences having a high degree of sequence similarity. For example, a plant-derived AK gene may be identified by searching databases such as those maintained by NCBI.

Binary vector pSIM1358 carries the AK gene with chloroplast signal peptide sequence fused to the Agp promoter. It also contains an expression cassette for the neomycin phosphotransferase (nptII) selectable marker gene. This vector was used to transform the potato variety Russet Burbank. Transcript analysis identified six lines that produced tubers expressing the AK gene. Tubers from four of these lines, 1358-1, 3, 20, and 25, were analyzed for amino acid composition. In two cases (1358-3 and 20), the transgenic tubers contained 16-25% lower amounts of asparagine than plants that had been transformed with a control vector only containing the nptII gene (Table 1).

A similar reduction in the accumulation of asparagine was observed in transgenic tubers from line 1359-23, which was transformed with pSIM1359 and overexpressed a codon-optimized version of the AK gene (Table 1). One line producing tubers that expressed an AK gene without signal peptide (1360-18) contained lower levels of asparagine. In all cases, the altered levels of asparagine were not associated with significant changes in the accumulation of other amino acids.

TABLE 1

| Plant | Asparagine (mg/100 g fresh weight) |
|---|---|
| Transgenic control | 2.05 ± 0.18 |
| 1358-1 | 1.82 |
| 1358-3 | 1.54 |
| 1358-20 | 1.72 |
| 1358-25 | 2.11 |
| 1359-6 | 1.99 |
| 1359-15 | 1.92 |
| 1359-17 | 1.94 |
| 1359-23 | 1.67 |
| 1360-18 | 1.51 |

### Example 3

### Overexpression of Glutamine-Rich Storage Proteins

It is possible to reduce asparagine levels by providing a sink for free glutamine. One example of such a sink is the glutamine-rich storage protein. Many crops produce large amounts of proteins such as glutenin in their storage organs. The sequence of one wheat glutenin gene is shown as SEQ ID NO: 20. Basically any glutamine-rich protein can be used. Such a gene can be fused to promoter that is highly active in tubers, and the resulting expression cassette can be introduced into potato. The resulting plants will produce tubers that accumulate glutenin or another glutamine-rich protein gene. This protein will represent more than 1% of the total protein fraction of potato tubers. By removing the amount of free glutamine, there will be less substrate available to produce asparagine.

A binary vector carrying the glutamine-rich storage protein fused to a promoter that is functional in tubers can be used to transform potato. Some of the transgenic plants will express the gene and produce the new storage protein. These plants will contain reduced levels of asparagine.

### Example 4

### Simultaneous Down-Regulated Expression of Genes Involved in Starch Degradation and Aspartate Biosynthetic Genes

This example demonstrates that the simultaneous down-regulated expression of genes involved in starch degradation and aspartate biosynthesis, respectively, in starchy tissues of a crop can lower acrylamide accumulation in a food obtained from heating these

Transgenic plants producing tubers with low levels of reducing sugars and asparate can be generated in two steps. First, plants are transformed with the P-DNA of binary vector pSIM371. This P-DNA contains two copies of a polynucleotide comprising fragments of the PPO (SEQ ID NO: 21), R1 (SEQ ID NO: 22), and phL (SEQ ID NO: 23) gene, inserted as inverted repeat between the Gbss promoter and Ubi3 terminator.

An Agrobacterium strain carrying both pSIM371 and the LifeSupport vector pSIM368, which contains expression cassettes for both the nptII and codA genes inserted between T-DNA borders, was used to infect 21,900 potato stem explants. After a two-day co-cultivation period, the infected explants were subjected for five days to kanamycin to select for transient nptII gene expression. To prevent the proliferation of cells containing stably integrated T-DNAs, explants were subsequently transferred to media containing 5-fluorocytosine (5FC). This chemical is converted into toxic 5-fluorouracil (5FU) by the codA gene product. A total of 3,822 shoots that survived the double selection were genotyped for presence of the P-DNA and absence of any foreign DNA from either T-DNA or plasmid backbone. This analysis identified 256 all-native DNA (intragenic) shoots that were allowed to root, planted into soil, and grown for six weeks in growth chambers. To screen for PPO activity, a catechol solution was pipetted onto the cut surfaces of harvested about 2-cM mini-tubers. Fourty-eight lined that were inhibited in catechol-induced tuber browning were grown in the greenhouse for three months to produce semi-mature tubers that were biochemically assessed for residual levels of PPO activity. This analysis demonstrated that employment of the PPO gene silencing construct lowered PPO activity by about 90%. Both the 48 intragenic lines and untransformed Ranger Russet and Russet Burbank control plants were subsequently propagated and grown in the field in Idaho, Aberdeen.

The mature tubers of all intragenic and control lines were analyzed for glucose levels after three and six-month of cold-storage. Most lines (43) displayed a greater reduction in cold-induced sweetening (about 60%) than obtained with control lines that had been silenced for only one of the starch-associated genes. French fries derived from the silenced tubers of plants 371-28 and 371-38 contained less than a third of the neurotoxin acrylamide that accumulated in control fries. Such a reduction was anticipated because acrylamide is largely derived from heat-induced reactions between the carbonyl group of reducing sugars and asparagine.

The sensory characteristics of modified French fries were evaluated by a panel of eight professionally trained individuals. French fries derived from tubers of the modified Ranger Russet displayed a better visual appearance than fries from either Ranger Russet or Russet Burbank. Furthermore, the intragenic fries displayed a significantly better overall aroma as sensed by the olfactory epithelium which is located in the roof of the nasal cavity. A similar trend was observed for tubers that had been stored for ten weeks at 4° C. In fact, the cold-stored intragenic lines 371-28, 30, 38, and 68 still met or exceeded the sensory attributes of fresh untransformed varieties.

One low sugar potato line can be retransformed with a T-DNA carrying a silencing cassette targeting the Aat2 gene. French fries from the kanamycin resistant double transformants will generally display further reduced levels of acrylamide. Thus, the double transformants produce tubers that can be used to obtain fries that (i) contain reduced levels of acrylamide and (ii) display enhanced sensory characteristics.

### Example 5

### Down-Regulated Expression of Glutamine Biosynthetic Genes

This example relates that the down-regulated expression of genes involved in glutamine biosynthesis in starchy tissues of a crop can lower acrylamide accumulation in a food obtained from heating these tissues.

Glutamine synthetase (GS) catalyzes the conversion of glutamate and ammonium into glutamine. This glutamine then functions as amino group donor for aspartate to produce asparagine. Overexpression of GS enhances nitrogen assimilation and reduces, in some cases, asparagine accumulation.

There are two versions of GS. One version is targeted to the chloroplast (see, for example, the gene encoding the potato chloroplast GS2 protein in SEQ ID 24). The second version is cytoplasmic. Potato genes encoding the cytoplasmic GS1A and GS1B protein are shown as SEQ ID NO: 25 and 26, respectively.

A silencing construct that lowers levels of both glutamine and asparagine comprises two copies of a DNA segment, each of which comprises fragments of both the GS1B gene (which is homologous to GS1A) and GS2, inserted as inverted repeat between regulatory elements that promote expression in tubers. The antisense copy of the DNA segment is shown as SEQ ID NO: 27. The sense component is somewhat larger to include a spacer sequence and shown in SEQ ID NO: 28.

Analyses of 25 lines containing independent insertions of a T-DNA carrying this silencing cassette will identify several plants that produce tubers containing reduced levels of glutamine and asparagine. Upon processing, the resulting fries or potato chips will contain reduced levels of acrylamide.

### Example 6

### Tuber-Specific Overexpression of a Lysine Feedback-Insensitive Dihydrodipicolinate Synthase

This example relates that the overexpression of a gene involved in aspartate metabolism in the starchy tissues of a crop lowers the amount of asparagine in these starchy tissues and, consequently, lowers the amount of acrylamide in a food obtained from heating these starchy tissues.

In order to modify the flux through the aspartate family pathway and enhance the accumulation of the essential amino acids such as free lysine while lowering accumulation of free asparagines in potato tubers, plants can be transformed with expression cassettes for the lysine feedback-insensitive forms of the dihydrodipicolinate synthase (DHPS) genes. The bacterial DHPS gene from E. coli shown in SEQ ID NO: 29 can to be fused to an upstream sequence encoding a chloroplast signal peptide to target the encoded protein to the plastid. Such a sequence can be obtained from, for instance, the pea rbcS gene (accession number J01257). The DHPS gene than can be expressed under a tuber specific promoter such as patatin, GBSS or AGP. The other example of a bacterial gene that can be used is a DHPS gene from Corynebacterium glutamicum that is shown in SEQ ID NO: 30.

Instead of bacterial DHPS gene a maize homologous sequence can be used, that has mutations in the DHPS gene coding region making the enzyme lysine feedback-less-sensitive due to amino acid substitutions at positions 157, 162 and 166. The examples of such sequences are shown in SEQ ID NO: 31-33.

Furthermore, the homologous to DHPS sequences can be isolated from the other plants species such as potato, sweet potato, wheat, and barley, and modified accordingly by the skilled in the art to make these sequences lysine feedback-insensitive. The modified in these way genes can be than expressed as described. Examples of a Solanum tuberosum DHPS sequence and a Triticum aestivum DHPS sequence are shown in SEQ ID NO: 34 and SEQ ID NO: 35.

Other promoters that can be used to drive DHPS gene expression in potato tubers can be selected from the group consisting of cold-inducible promoters and flavonoid-3′-mono-oxygenase (Fmo) promoters. One Fmo promoter is shown in SEQ ID NO: 19. This promoter is active in semi-mature and mature tubers but not in mini-tubers.

For any DHPS gene, there may be other sequences having a high degree of sequence similarity. For example, a plant-derived DHPS gene may be identified by searching databases such as those maintained by NCBI.

A binary vector carrying the DHPS gene fused to a promoter that is functional in tubers can be used to transform potato. Tubers of some of the transgenic plants will express the DHPS gene. These plants will contain reduced levels of asparagine.

### Example 7

### Down-Regulated Expression of a Mitochondrial Malate Dehydrogenase

The sequence of the potato mitochondrial malate dehydrogenase (MDH) gene is shown as SEQ ID NO: 36. A silencing construct targeting the expression of this gene in tubers comprises two copies of an MDH gene fragment inserted as inverted repeat between regulatory elements that promote expression in tubers. An example of a sense MDH gene fragment is shown in SEQ ID NO: 37. A slightly larger antisense fragment, which comprises sequences that function as internal spacer, is shown in SEQ ID NO: 38.

A binary vector carrying the MDH silencing construct can be used to transform potato. Tubers of some of the transgenic plants will express the MDH gene at levels that are lower than those in untransformed plants. These plants will contain reduced levels of asparagine.

### Example 8

### Multi-Gene Approaches

Any of the examples described above, or embodiments disclosed in the specification, can be combined to enhance the asparagine-lowering effect. They can also be combined with strategies that lower the accumulation of reducing sugars during cold storage. Apart from above-described methods based on silencing R1 and PhL, cold-induced sweetening can be lowered by overexpressing a vacuolar invertase inhibitor (Greiner et al., Ectopic expression of a tobacco invertase inhibitor homolog prevents cold-induced sweetening of potato tubers, Nat Biotechnol 17: 708-711, 1999) or overexpressing SnRK1 (McKibben et al., Production of high-starch, low-glucose potatoes through over-expression of the metabolic regulator SnRK1. Plant Biotechnol J 4: 409-418).

Furthermore, it is possible to lower asparagine levels and, simultaneously, increase the expression involved in genes that have a negative effect of the accumulation of reducing sugars. For instance, plants can be transformed with a transfer DNA that contains two expression cassettes. The first expression cassette is designed to down-regulate the expression of glutamine synthetase genes in tubers, and the second gene is meant to overexpress genes such as Dofl (Yanagisawa et al. Metabolic engineering with Dofl transcription factor in plants: Improved nitrogen assimilation and growth under low-nitrogen conditions. Proc Natl Acad Sci USA 101: 7833-7838, 2004), SnRk1 (McKibbin et al., Production of high-starch, low-glucose potatoes through over-expression of the metabolic regulator SnRK1. Plant Biotechnol J 4: 409-418, 2006), and MDH (Nunes-Nesi et al., Enhanced photosynthetic performance and growth as a consequence of decreasing mitochondrial malate dehydrogenase activity in transgenic tomato plants. Plant Physiol 137: 611-622, 2005).

### Sequences

| Potato Aatl gene | |
|---|---|
| SEQ ID 1 | |
| AAAAGCCGTTGATAAACCATTCGTTCGCACTGACGGCTCGCTCAGTCCACTACTCTTACGTCTCCTATATATAAAG | |
| TCCATCTCTATCAACCACTTCGACAAACACGCCATCTTCCTCTCCGATTTCTCTAAATACTCTCTCCGCTCCGGCC | |
| ATGGCGATCCGTGCCGCGATTTCCGGTAGTCATCTCAAGCTTAGCTCGTCGTCGCTCGGAGCGCGATCCTTGTCGT | |
| CGTGGTGGCGGAACGTTGAGCCGGCACCTAAAGATCCTATCCTCGGCGTTACTGAAGCTTTCCTCGCCGATCCTAG | |
| CCCTGATAAAGTCAATGTTGGCGTTGGAGCTTACAGGGATGACAATGGAAAACCCGTGGTACTGGAGTGTGTCAGA | |
| GAAGCAGAGCGAAGGATCGCTGGCGGTTTCAACATGGAATATCTTCCTATGGGAGGTAGTGTCAACATGATCCAGG | |
| AGTCGCTGAAGTTAGCCTATGGGGAGAACTCAGACTTGATAAAAGATAAACGCATTGCTGCAATTCAAGCATTATC | |
| TGGAACTGGCGCATGCCGAATTTTTGCGGACTTCCAAAAGCGCTTTTGTCCTGATTCACAGATTTATATTCCAGTT | |
| CCTACATGGTCTAATCATCATAACATTTGGAGAGATGCTCATGTCCCTGAGAGAACATATCACTATTATCATGCTG | |
| AATCAAAGGGCTTGGACTTTGCTGCAATGATGGATGATATAAAGAATGCCCCAAAAGGATCATTCTTTCTGCTTCA | |
| TGCTTGTGCTCATAATCCTACTGGGGTGGATCCGACAGAGGAACAATGGAGGGAGATCTCGCACCAGTTCAAGGTG | |
| AAGGGACATTTTGCTTTGTTTGATATGGCCTATCAAGGATTTGCAAGTGGGTATCCAGAGAAGGATGCTAAGGCAA | |
| TCAGGATATTTCTTGAGGATGGTCATCCGATAGCGTGTGCCCAATCATATGCAAAAAATATGGGGCTATATGGCCA | |
| GAGAGTTGGTTGCCTAAGTGTTGTCTGTGAGGATGAAAAACAAGCAGTGGCAGTGAAAAGTCAGTTGCAGCAACTT | |
| GCCAGGCCCATGTACAGTAATCCACCTGTTCATGGTGCTCTTGTTGTTTCTACCATCCTTGGAGATCCAAACTTGA | |
| AAAATCTATGGCTCGGGGAAGTGAAGGGCATGGCTGATCGTATCATCGGGATGAGAACTGCTTTAAGGGAAAATCT | |
| TGAGAAGTTGGGCTCACCTCTATCTTGGGAACACATAACCAACCAGATTGGCATGTTCTGCTACAGTGGGATGACA | |
| CCGGAACAAGTAGACCGATTGACAAAAGAGTATCACATCTACATGACTCGTAATGGTCGTATCAGTATGGCAGGAG | |
| TTACTACTGGAAATGTTGGTTACTTGGCAAACGCTATTCATGAGGTTACTAAATCTGCTTAAGCTTTAACATCATG | |
| GATTCAATAATCAGTTACGGAAAGGCTGTGGAAATTCATTTTTTGTGATCGGAAAGATCACAACATGTTGTAATAG | |
| TTGAGTTAAAACAATCGTACTGAGGGGAGGGGGGAATAAATAGAACCATTTTAAGGGATAGAAGTCATTAGCAGCC | |
| GTCGAGGTGGAGTTGTAGTTATTTTAGGGTACATTACAGACATTAATCATCTCTAGTGTCAAGGAACTGCTGTTTT | |
| TCATATACAACAGCGTAGTTACATGATATATATGCCAACATTTTGAAGTATTGTTACTGTTTTATGGCGTCATGGA | |
| TGGAAGTAATTATTG. | |
| Potato Aat2 gene | |
| SEQ ID 2 | |
| CTCCATAACATCGTTGAACTTGTTCTCTGAAATTCCAACTCCAATGGCAGATTCTGTGTTTGCTCATGTTGTTCGT | |
| GCTCCTGAAGATCCCATCTTAGGGGTAACAGTTGCTTATAACAAAGATAGCAGCCCAGTCAAAGTGAATTTGGGTG | |
| TTGGCGCATATCGAACTGAGGAAGGAAAGCCCCTTGTTCTTAATGTCGTGAGACGAGCTGAACAAATGCTTGTCAA | |
| TGACACGTCTCGCGTGAAGGAATATCTCTCAATTACTGGACTTGCGGATTTTAACAAATTGAGTGCCAAGCTTATA | |
| TTTGGTGCTGACAGCCCTGCCATTAAAGAGAATAGGGTGACCACTGTTCAGTGCTTGTCGGGCACTGGTTCTTTGA | |
| GGGTTGGTGCTGAGTTTCTGGCCAGGCATTATCATGAACATACTATATATATACCACAGCCAACATGGGGAAACCA | |
| TCCTAAGATTTTCACTTTAGCTGGGCTTTCAGTAAAATATTATCGTTACTATGACCCAGCAACACGAGGCCTGGAT | |
| TTTCAAGGATTATTGGATGATCTTACGGCTGCACCCTCTGGTGCAATAGTTCTTCTCCATGCTTGTGCTCATAACC | |
| CAACTGGCGTTGATCCAACAATTGACCAGTGGGAAAAAATCAGGAAGTTGATGAGGTCCAAGGGGTTGTTACCTTT | |
| CTTTGACAGTGCTTATCAGGGTTTTGCCAGTGGGAACCTAGATGCAGATGCACAATCTGTTCGCATGTTTGTGGCT | |
| GATGGTGGTGAATGTCTTGCAGCTCAGAGTTATGCCAAAAACATGGGACTGTATGGAGAGCGTGTCGGTGCCCTTA | |
| GCATTGTTTGCAAAGATAGCAATGTTGCAAGCAAAGTCGAAAGCCAGCTAAAGATGGTTATAAGGCCAATGTACTC | |
| AAATCCACCGATTCATGGTGCGTCTATTGTTGCCACTATACTCAAGGACAGACAAATGTATGACGAATGGACAGTT | |
| GAGCTGAAAGCAATGGCCGACCGAATTATTAGCATGCGCCAGCAACTCTATGATGCCATGCAAGCTCGAGGTACTC | |
| CAGGTGATTGGAGTCATATCATCAAGCAAATTGGGATGTTTACTTTCACAGGATTGAATGTTGAGCAAGTCTCGTT | |
| CATGACTAGAGAGTATCACATTTACATGACATCTGATGGGAGAATTAGCATGGCAGGTCTTAGTTCTCGGACAGTT | |
| CCTCATCTTGCTGATGCCATGCATGCTGCTGTTACTAGGGCGGCCTAGATTAGTTGCATTCAAGAGCAGTTTTATT | |
| AATATTGAATCTACTTTCCATGATCGCCTCCTTATCCTTTGATGTTAAATAAGACGAATGTGTCTATGTAGACATA | |
| ATTTATATTTGGAAATTTCATTCTAAAGGAATTTGATGAGGTCTTCTGCTAAGGGG. | |
| Potato Aat3 gene | |
| SEQ ID 3 | |
| GATAAAGATAGGCGTACTATAGCAAGCAAAAGTTCCATACTACTAAAATGCACACACAACAATCACCGTCACCCTC | |
| CGCCGATCGGAGGTTGAGTGTTCTGGCGAGGCACCTTGAACCGTCGTCCTCCACCGCCGTTGAAGGTCACAGCAAC | |
| CACTCCATTGTCGGTGCTCCTACATCAGGATATGATGGAAAACAGTCTGTATTCTCTCACATCGTTCGCGCTCCCG | |
| AAGATCCTATTCTCGGCGTTACTGTTGCATACAACAAAGATACCAGCCCCGTGAAGTTGAACTTGGGAGTTGGTGC | |
| ATATAGGACAGAGGAAGGAAAACCTCTTGTTTTGAATGTTGTTAGAAAAGCAGAACAGCTACTAGTAAATGATAGG | |
| TCTCGCATAAAAGAGTACCTTCCAATTGTTGGGCTGGCAGACTTCAATAAATTGAGTGCTAAGCTGATACTTGGTG | |
| CTGACAGCCCTGCTATTCAAGAGAACAGAGTAACAACTGTGCAGTGCTTGTCTGGCACAGGCTCATTGAGGGTTGG | |
| AGGTGAATTTTTGGCTCAACATTATCATCAACGTACTATTTATATTCCCCAACCGACATGGGGAAACCACCCAAAA | |
| ATTTTCACTTTAGCTGGATTGTCAGTTAAGAGTTACCGCTACTATGATCCAGCAACCCGTGGACTCCATTTTCAAG | |
| GTTTGTTGGAAGACCTTGGATCTGCTCCGTCAGGAGCGATAGTGTTACTTCACGCTTGTGCACATAACCCCACTGG | |
| TGTGGATCCAACCAAAGATCAGTGGGAGCAAATTAGGAAATTGATGAGATCAAAAGGATTGCTACCCTTCTTTGAT | |
| AGTGCATATCAGGGGTTTGCTAGTGGAAGCCTAGATATAGATGCACAGTCTGTTCGCATGTTTGTGGCAGATGGAG | |
| GTGAAGTACTTGTTGCTCAAAGTTATGCAAAGAATATGGGGCTTTATGGTGAACGTGTCGGAGCTTTAAGCATAGT | |
| GTGCAGAAATGCTGATGTAGCTAGCAGAGTTGAGAGCCAGTTTGAAGTTGGGGTGTATTTAGGGCCAAATGGTATT | |
| CCCCAAGTTCCCACCAAATTTCCACGGGTGGCGTCAACTAGTTTGCTTACAAATCCTCCAAAGACAGGAAAACTTG | |
| TTACCACTGAATGGCACTCTTGGAATTTGAAAAGCCGATGGGTTGATCGAATTCTTCCAG. | |
| Potato Aat4 gene | |
| SEQ ID 4 | |
| GCTAATTTTACAATACAAGCAAAAAAACAGAACAGAAGAGAGCAAGAAAGATGAACATGTGGAGTAGACATGTTAG | |
| ATGGCTGTCTACCTCAGTAGCAACAAATAGGAAAAACAATATGGGATGGTGGGACCATGTGCAGCCAGCTCCTAAA | |
| GACCCAATCACCAGTGTCACTCAGGCTTTTCTTTCTGACCCAACTCCTCTCAAGCTCAATCTTGGGAGCTTACAGA | |
| GATGATAAAGGAAAACCAGTAACTCTTGAGTGTGTTAGAAGAGCTGCTGAAAAGATTTCTGGCTGTGAAATCTTGG | |
| AATCAACTAAAGCAACAACAAAATCAAAGTTTGTGCAGGACTGTGTTAAATTGGCTTATGGAAATGACTCTAGTGT | |
| TGTTAGAGAAGACAGGTTTGCTGGTGTTCCAGCTCTTTCTGGGACTGGTGCGTGCCGGCTGTTTGCGGAGTTCCAA | |
| AGGCGCTTCTATCCTGACTCACAAATGTTTCTACCAATGCCAACTTGGTCCAACCACCACGACATTTGGAGGGATT | |
| CTCAAGTTTGTACAAGAACCTACCACTATTATGATCCTGGTACAAAAGGATTGCGATTTCAAGCCCTCGTAAATGA | |
| TATCAAGAATGCTCCAGATCGTTCCTTTTTCTTACTTCATCCTTGTGCTCATACCCCAACTGGTGTCGACCCCAAC | |
| TATGAGCAGTGGAAAGAAATCTCACACATATTTAAGATCAAGAATCATTTTCCTTTTTTTGATATGGCTTATCAAG | |
| GAATTGCTAGCGGAGATGTTGAGAGAGATGCCACTGCTATTCGAATATTTCTAGAAGATGGACATCTATTGGGCTG | |
| TGCCCAATCTTTTTCCAAAAACATGGGCTTGTATGAACACCGAGTAGGTTGTGTCAGTATTGTCTCCTGGGATGAT | |
| AAGCAGGCCACAGCAATCAAGAGCCAGTTGCAGCGAATTGTCAGGGCAATGTACAGTAGCTCTCCGGTTCATGGCC | |
| CATTATTGGTGTCAACAATCTTGAATGATGCTGATTTAAAGGCACTTTGGGAAGAGGAAGTTAAGGTCATGGTGGA | |
| TCGCTTAATCAGTATGAGAATCACATTACGTCAAACTCTTGAGGAGTTGAACTCATCTTCGAGCTGGGAACATATA | |
| ACGAAGCAGGTTGGAATGTTTTACTTCTCTGGTTTATCCCCTGAGGAAGTTAACCACTTGCAGAGGGATTTTCATA | |
| TATACATGACTAACGATGGGCGCATCAGCATGGCAGGGGTAACAAGAAGCAATGTAGACTACTTGGCAAGTGCAAT | |
| CCATGAAGTTACCAGTGTAAATAATAGCAACTGTATTGATTCAAAGCATTTTGAATTTGTAGTTTAGCAATTCTTA | |
| TTGAGATCTTGTGCAGATAACCATTGTGTAATACTGTTAGAGGTTGTTCGGTAGGATGTATTAGAGAAAATAATAC | |
| ATGTATACTTATTCAGTACTATTCTTATACATAACAATCAATGATATTAGCAATATAAGGGTTATTAAT. | |
| Potato Aat5 gene | |
| SEQ ID 5 | |
| TTACCATCGATCAGTTCTTCCCTCTGTGGTAGCTTTCCTGTAAACCCAAAGTCCCGAAACCCTTGTATGCCCTCTT | |
| CTTCTTTTGCGATGGCTTCCACAATGTTCTCTCTAGCTTCTGTCACACCTTCAGCTTCAATTTCCTTGCAAGATAA | |
| TCTCAAGCCAAAGCTGAAGCTAGGGACTTCTAGCCAAAGGGCCTTTCCCAGGAAGGACTTCATGAAGGCAAAGTCA | |
| AATGGTCGGACTACTATGGCTGTTGCTGTTAACGTCTCTCGATTTGAGGGAATAACAATGGCTCCCCCTGACCCCA | |
| TTCTTGGAGTCTCTGAAGCATTCAAGGCTGATACAAATGAACTGAAGCTTAACCTTGGAGTTGGAGCTTACCGCAC | |
| AGAGGATCTTCAACCATATGTCCTCAATGTTGTTAAGAAAGCAGAGAACCTTATCCTGGAGAGAGGAGAAAACAAA | |
| GAGTATCTTCCAATAGAAGGTTTGGCTGCATTCAACAAAGTCACAGCAGAGTTATTGTTTGGAAAAGATAACCTGG | |
| TGCTTCAGCAACAAAGGGTGGCTACGATTCAAGGTCTATCAGGAACTGGGTCATTGCGTATTGCTGCAGCACTGAT | |
| AGAGCGTTACTTCCCTGGCTCTAAGATTTTGATATCATCTCCAACATGGGGAAATCATAAAAACATTTTTAATGAT | |
| GCCAGAGTACCTTGGTCTGAATATCGATATTATGATCCCAAAACTGTTGGCCTTGACTTTGTTGGGATGATAGAAG | |
| ATATAAACGGCTGCCCCAGAAAGGATCACCTTTATCCTTGCTCCACGGCTGTGCACACAACCCAACTGGTATTGAT | |
| TCCCACACCTTGAGCAATGGGAAAAGATCGCTGATGGTATTCAGGAGAAGAACCACATTCCATTTTTTGATGTTGC | |
| CTACCAAGGATTTTGCAGTGGCAGCCTTGACGAAGATGCCTCATCTGTGAGAATGTTTGCTGCACGTGGTATGGAG | |
| CTTTTGGGTGCTCAATCATATAGTAAAAATCTGGGTCTCTATGGAGAAGGATGGGGGCTATAAATGTCTTTGCTCA | |
| TCTGCTGA. | |
| Fragment of potato Aatl gene | |
| SEQ ID 6 | |
| TGCTTCATGCTTGTGCTCATAATCCTACTGGGGTGGATCCGACAGAGGAACAATGGAGGGAGATCTCGCACCAGTT | |
| CAAGGTGAAGGGACATTTTGCTTTGTTTGATATGGCCTATCAAGGATTTGCAAGTGGGTATCCAGAGAAGGATGCT | |
| AAGGCAATCAGGATATTTCTTGAGGATGG. | |
| Fragment of potato Aat2 gene | |
| SEQ ID 7 | |
| TTCTCCATGCTTGTGCTCATAACCCAACTGGCGTTGATCCAACAATTGACCAGTGGGAAAAAATCAGGAAGTTGAT | |
| GAGGTCCAAGGGGTTGTTACCTTTCTTTGACAGTGCTTATCAGGGTTTTGCCAGTGGGAACCTAGATGCAGATGCA | |
| CAATCTGTTCGCATGTTTGTGGCTGATGG. | |
| Fragment of potato Aat3 gene | |
| SEQ ID 8 | |
| TACTTCACGCTTGTGCACATAACCCCACTGGTGTGGATCCAACCAAAGATCAGTGGGAGCAAATTAGGAAATTGAT | |
| GAGATCAAAAGGATTGCTACCCTTCTTTGATAGTGCATATCAGGGGTTTGCTAGTGGAAGCCTAGATATAGATGCA | |
| CAGTCTGTTCGCATGTTTGTGGCAGATGG. | |
| Fragment of potato Aat4 gene | |
| SEQ ID 9 | |
| TACTTCATCCTTGTGCTCATACCCCAACTGGTGTCGACCCCAACTATGAGCAGTGGAAAGAAATCTCACACATATT | |
| TAAGATCAAGAATCATTTTCCTTTTTTTGATATGGCTTATCAAGGAATTGCTAGCGGAGATGTTGAGAGAGATGCC | |
| ACTGCTATTCGAATATTTCTAGAAGATGG. | |
| Fragment of potato Aat5 gene | |
| SEQ ID 10 | |
| TGCTCCACGGCTGTGCACACAACCCAACTGGTATTGATTCCCACACCTTGAGCAATGGGAAAAGATCGCTGATGGT | |
| ATTCAGGAGAAGAACCACATTCCATTTTTTGATGTTGCCTACCAAGGATTTTGCAGTGGCAGCCTTGACGAAGATG | |
| CCTCATCTGTGAGAATGTTTGCTGCACGTGG. | |
| Promoter of the Agp gene | |
| SEQ ID 11 | |
| CAAGTGTCTGAGACAACCAAAACTGAAAGTGGGAAACCAAACTCTAAGTCAAAGACTTTATATACAAAATGGTATA | |
| AATATAATTATTTAATTTACTATCGGGTTATCGATTAACCCGTTAAGAAAAAACTTCAAACCGTTAAGAACCGATA | |
| ACCCGATAACAAAAAAAATCTAAATCGTTATCAAAACCGCTAAACTAATAACCCAATATTGATAAACCAATAACTT | |
| TTTTTATTCGGGTTATCGGTTTCAGTTCTGTTTGGAACAATCCTAGTGTCCTAATTATTGTTTTGAGAACCAAGAA | |
| AACAAAAACTTACGTCGCAAATATTTCAGTAAATACTTGTATATCTCAGTGATAATTGATTTCCAACATGTATAAT | |
| TATCATTTACGTAATAATAGATGGTTTCCGAAACTTACGCTTCCCTTTTTTCTTTTGCAGTCGTATGGAATAAAAG | |
| TTGGATATGGAGGCATTCCCGGGCCTTCAGGTGGAAGAGACGGAGCTGCTTCACAAGGAGGGGGTTGTTGTACTTG | |
| AAAATGGGCATTTATTGTTCGCAAACCTATCATGTTCCTATGGTTGTTTATTTGTAGTTTGGTGTTCTTAATATCG | |
| AGTGTTCTTTAGTTTGTTCCTTTTAATGAAAGGATAATATCTGTGCAAAAATAAGTAAATTCGGTACATAAAGACA | |
| TTTTTTTTTGCATTTTCTGTTTATGGAGTTGTCAAATGTGAATTTATTTCATAGCATGTGAGTTTCCTCTCCTTTT | |
| TCATGTGCCCTTGGGCCTTGCATGTTTCTTGCACCGCAGTGTGCCAGGGCTGTCGGCAGATGGACATAAATGGCAC | |
| ACCGCTCGGCTCGTGGAAAGAGTATGGTCAGTTTCATTGATAAGTATTTACTCGTATTCGGTGTTTACATCAAGTT | |
| AATATGTTCAAACACATGTGATATCATACATCCATTAGTTAAGTATAAATGCCAACTTTTTACTTGAATCGCCGAA | |
| TAAATTTACTTACGTCCAATATTTAGTTTTGTGTGTCAAACATATCATGCACTATTTGATTAAGAATAAATAAACG | |
| ATGTGTAATTTGAAAACCAATTAGAAAAGAAGTATGACGGGATTGATGTTCTGTGAAATCACTGGTAAATTGGACG | . |
| GACGATGAAATTTGATCGTCCATTTAAGCATAGCAACATGGGTCTTTAGTCATCATCATTATGTTATAATTATTTT | |
| CTTGAAACTTGATACACCAACTTTCATTGGGAAAGTGACAGCATAGTATAAACTATAATATCAATTCTGGCAATTT | |
| CGAATTATTCCAAATCTCTTTTGTCATTTCATTTCCTCCCCTATGTCTGCAAGTACCAATTATTTAAGTACAAAAA | |
| ATCTTGATTAAACAATTTATTTTCTCACTAATAATCACATTTAATCATCAACGGTTCATACACGTCTGTCACTCTT | |
| TTTTTATTCTCTCAAGCGCATGTGATCATACCAATTATTTAAATACAAAAAATCTTGATTAAACAATTCAGTTTCT | |
| CACTAATAATCACATTTAATCATCAACGGTTCATACACATCCGTCACTCTTTTTTTATTCTCTCAAGCGCATGTGA | |
| TCATACCAATTATTTAAATACAAAAAATCTTGATTAAACAATTCATTTTCTCACTAATAATCACATTTAATCATCA | |
| ACGGTTTATACACGTCCGCCACTCTTTTTTTATTCTCTCAAGCGTATGTGATCATATCTAACTCTCGTGCAAACAA | |
| GTGAAATGACGTTCACTAATAAATAATCTTTTGAATACTTTGTTCAGTTTAATTTATTTAATTTGATAAGAATTTT | |
| TTTATTATTGAATTTTTATTGTTTTAAATTAAAAATAAGTTAAATATATCAAAATATCTTTTAATTTTATTTTTGA | |
| AAAATAACGTAGTTCAAACAAATTAAAATTGAGTAACTGTTTTTCGAAAAATAATGATTCTAATAGTATATTCTTT | |
| TTCATCATTAGATATTTTTTTTAAGCTAAGTACAAAAGTCATATTTCAATCCCCAAAATAGCCTCAATCACAAGAA | |
| ATGCTTAAATCCCCAAAATACCCTCAATCACAAGACGTGTGTACCAATCATACCTATGGTCCTCTCGTAAATTCCG | |
| ACAAAATCAGGTCTATAAAGTTACCCTTGATATCAGTATTATAAAACTAAAAATCTCAGCTGTAATTCAAGTGCAA | |
| TCACACTCTACCACACACTCTCTAGTAGAGAGATCAGTTGATAACAAGCTTGTTAACG. | |
| Promoter of the Gbss gene | |
| SEQ ID 12 | |
| CGAACCATGCATCTCAATCTTAATACTAAAAAATGCAACAAAATTCTAGTGGAGGGACCAGTACCAGTACATTAGA | |
| TATTATCTTTTATTACTATAATAATATTTTAATTAACACGAGACATAGGAATGTCAAGTGGTAGCGGTAGGAGGGA | |
| GTTGGTTCAGTTTTTTAGATACTAGGAGACAGAACCGGAGGGGCCCATTGCAAGGCCCAAGTTGAAGTCCAGCCGT | |
| GAATCAACAAAGAGAGGGCCCATAATACTGTCGATGAGCATTTCCCTATAATACAGTGTCCACAGTTGCCTTCCGC | |
| TAAGGGATAGCCACCCGCTATTCTCTTGACACGTGTCACTGAAACCTGCTACAAATAAGGCAGGCACCTCCTCATT | |
| CTCACACTCACTCACTCACACAGCTCAACAAGTGGTAACTTTTACTCATCTCCTCCAATTATTTCTGATTTCATGC | |
| ATGTTTCCCTACATTCTATTATGAATCGTGTTATGGTGTATAAACGTTGTTTCATATCTCATCTCATCTATTCTGA | |
| TTTTGATTCTCTTGCCTACTGAATTTGACCCTACTGTAATCGGTGATAAATGTGAATGCTTCCTCTTCTTCTTCTT | |
| CTTCTCAGAAATCAATTTCTGTTTTGTTTTTGTTCATCTGTAGCTTGGTAGATTCCCCTTTTTGTAGACCACACAT | |
| CACG. | |
| terminator of the Ubiquitin-3 gene | |
| SEQ ID 13 | |
| TTGATTTTAATGTTTAGCAAATGTCCTATCAGTTTTCTCTTTTTGTCGAACGGTAATTTAGAGTTTTTTTTGCTAT | |
| ATGGATTTTCGTTTTTGATGTATGTGACAACCCTCGGGATTGTTGATTTATTTCAAAACTAAGAGTTTTTGCTTAT | |
| TGTTCTCGTCTATTTTGGATATCAATCTTAGTTTTATATCTTTTCTAGTTCTCTACGTGTTAAATGTTCAACACAC | |
| TAGCAATTTGGCTGCAGCGTATGGATTATGGAACTATCAAGTCTGTGGGATCGATAAATATGCTTCTCAGGAATTT | |
| GAGATTTTACAGTCTTTATGCTCATTGGGTTGAGTATAATATAGTAAAAAAATAGG. | |
| Aspartate kinase gene | |
| SEQ ID 14 | |
| GGATCCATGTCTGAAATTGTTGTCTCCAAATTTGGCGGTACTAGCGTAGCCGATTTTGACGCCATGAACCGCAGCG | |
| CTGATATTGTGCTTTCTGATGCCAACGTGCGTTTAGTTGTCCTCTCGGCTTCTGCTGGTATCACTAATCTGCTGGT | |
| CGCTTTAGCTGAAGGACTGGAACCTTGCGAGCGATTCGAAAAACTCGACGCTATCCGCAACATCCAGTTTGCCATT | |
| CTGGAACGTCTGCGTTACCCGAACGTTATCCGTGAAGAGATTGAACGTCTGCTGGAGAACATTACTGTTCTGGCAG | |
| AAGCGGCGGCGCTGGCAACGTCTCCGGCGCTGACAGATGAGCTGGTCAGCCACGGCGAGCTGATGTCGACCCTGCT | |
| GTTTGTTGAGATCCTGCGCGAACGCGATGTTCAGGCACAGTGGTTTGATGTGCGTAAAGTGATGCGTACCAACGAC | |
| CGATTTGGTCGTGCAGAGCCAGATATAGCCGCGCTGGCGGAACTGGCCGCGCTGCAGCTGCTCCCACGTCTCAATG | |
| AAGGCTTAGTGATCACCCAGGGATTTATCGGTAGCGAAAATAAAGGTCGTACAACGACGCTTGGCCGTGGAGGCAG | |
| CGATTATACGGCAGCCTTGCTGGCGGAGGCTTTACACGCATCTCGTGTTGATATCTGGACCGATGTCCCGGGCATC | |
| TACACCACCGATCCACGTGTAGTTTCCGCAGCAAAACGCATTGATGAAATCGCGTTTGCCGAAGCGGCAGAGATGG | |
| CAACTTTTGGTGCAAAAGTACTGCATCCGGCAACGTTGCTACCCGCAGTACGCAGCGATATCCCGGTCTTTGTCGG | |
| CTCCAGCAAAGACCCACGCGCAGGTGGTACGCTGGTGTGCAATAAAACTGAAAATCCGCCGCTGTTCCGCGCTCTG | |
| GCGCTTCGTCGCAATCAGACTCTGCTCACTTTGCACAGCCTGAATATGCTGCATTCTCGCGGTTTCCTCGCGGAAG | |
| TTTTCGGCATCCTCGCGCGGCATAATATTTCGGTAGACTTAATCACCACGTCAGAAGTGAGCGTGGCATTAATCCT | |
| TGATACCACCGGTTCAACCTCCACTGGCGATACGTTGCTGACACAATCTCTGCTGATGGAGCTTTCCGCACTGTGT | |
| CGGGTGGAGGTGGAAGAAGGTCTGGCGCTGGTCGCGTTGATTGGCAATGACCTGTCAAAAGCGTGCGCCGTTGGCA | |
| AAGAGGTATTCGGCGTACTGGAACCGTTCAACATTCGCATGATTTGTTATGGCGCATCCAGCCATAACCTGTGCTT | |
| CCTGGTGCCCGGCGAAGATGCCGAGCAGGTGGTGCAAAAACTGCATAGTAATTTGTTTGAGTGAGAATTC. | |
| Aspartate kinase gene fused to a signal peptide sequence | |
| SEQ ID 15 | |
| GGATCCTATTTTTACAACAATTACCAACAACAACAAACAACAAACAACATTACAATTACTATTTACAATTACAATG | |
| GCTTCTATGATATCCTCTTCCGCTGTGACAACAGTCAGCCGTGCCTCTAGGGGGCAATCCGCCGCAGTGGCTCCAT | |
| TCGGCGGCCTCAAATCCATGACTGGATTCCCAGTGAAGAAGGTCAACACTGACATTACTTCCATTACAAGCAATGG | |
| TGGAAGAGTAAAGTGCATGCATGTCTGAAATTGTTGTCTCCAAATTTGGCGGTACTAGCGTAGCCGATTTTGACGC | |
| CATGAACCGCAGCGCTGATATTGTGCTTTCTGATGCCAACGTGCGTTTAGTTGTCCTCTCGGCTTCTGCTGGTATC | |
| ACTAATCTGCTGGTCGCTTTAGCTGAAGGACTGGAACCTTGCGAGCGATTCGAAAAACTCGACGCTATCCGCAACA | |
| TCCAGTTTGCCATTCTGGAACGTCTGCGTTACCCGAACGTTATCCGTGAAGAGATTGAACGTCTGCTGGAGAACAT | |
| TACTGTTCTGGCAGAAGCGGCGGCGCTGGCAACGTCTCCGGCGCTGACAGATGAGCTGGTCAGCCACGGCGAGCTG | |
| ATGTCGACCCTGCTGTTTGTTGAGATCCTGCGCGAACGCGATGTTCAGGCACAGTGGTTTGATGTGCGTAAAGTGA | |
| TGCGTACCAACGACCGATTTGGTCGTGCAGAGCCAGATATAGCCGCGCTGGCGGAACTGGCCGCGCTGCAGCTGCT | |
| CCCACGTCTCAATGAAGGCTTAGTGATCACCCAGGGATTTATCGGTAGCGAAAATAAAGGTCGTACAACGACGCTT | |
| GGCCGTGGAGGCAGCGATTATACGGCAGCCTTGCTGGCGGAGGCTTTACACGCATCTCGTGTTGATATCTGGACCG | |
| ATGTCCCGGGCATCTACACCACCGATCCACGTGTAGTTTCCGCAGCAAAACGCATTGATGAAATCGCGTTTGCCGA | |
| AGCGGCAGAGATGGCAACTTTTGGTGCAAAAGTACTGCATCCGGCAACGTTGCTACCCGCAGTACGCAGCGATATC | |
| CCGGTCTTTGTCGGCTCCAGCAAAGACCCACGCGCAGGTGGTACGCTGGTGTGCAATAAAACTGAAAATCCGCCGC | |
| TGTTCCGCGCTCTGGCGCTTCGTCGCAATCAGACTCTGCTCACTTTGCACAGCCTGAATATGCTGCATTCTCGCGG | |
| TTTCCTCGCGGAAGTTTTCGGCATCCTCGCGCGGCATAATATTTCGGTAGACTTAATCACCACGTCAGAAGTGAGC | |
| GTGGCATTAATCCTTGATACCACCGGTTCAACCTCCACTGGCGATACGTTGCTGACACAATCTCTGCTGATGGAGC | |
| TTTCCGCACTGTGTCGGGTGGAGGTGGAAGAAGGTCTGGCGCTGGTCGCGTTGATTGGCAATGACCTGTCAAAAGC | |
| GTGCGCCGTTGGCAAAGAGGTATTCGGCGTACTGGAACCGTTCAACATTCGCATGATTTGTTATGGCGCATCCAGC | |
| CATAACCTGTGCTTCCTGGTGCCCGGCGAAGATGCCGAGCAGGTGGTGCAAAAACTGCATAGTAATTTGTTTGAGT | |
| GAGAATTC. | |
| Codon-optimized aspartate kinase gene | |
| SEQ ID 16 | |
| GGATCCTATTTTTACAACAATTACCAACAACAACAAACAACAAACAACATTACAATTACTATTTACAATTACAATG | |
| GCTTCTATGATATCCTCTTCCGCTGTGACAACAGTCAGCCGTGCCTCTAGGGGGCAATCCGCCGCAGTGGCTCCAT | |
| TCGGCGGCCTCAAATCCATGACTGGATTCCCAGTGAAGAAGGTCAACACTGACATTACTTCCATTACAAGCAATGG | |
| TGGAAGAGTAAAGTGCATGCATGTCAGAAATAGTCGTAAGCAAATTTGGTGGTACTTCTGTAGCTGATTTTGATGC | |
| TATGAACAGATCTGCTGATATAGTTTTATCAGACGCTAATGTACGTCTCGTTGTTTTGAGTGCATCCGCCGGTATC | |
| ACAAACTTGTTAGTCGCATTGGCAGAAGGTCTTGAACCATGTGAAAGATTTGAGAAACTTGACGCCATTAGAAATA | |
| TCCAATTCGCCATACTAGAAAGACTTAGATACCCAAACGTAATTCGTGAAGAAATAGAGCGTCTTTTAGAAAATAT | |
| AACAGTTCTTGCCGAAGCTGCTGCCCTTGCTACATCCCCAGCTCTTACTGATGAATTAGTGTCACACGGAGAATTA | |
| ATGTCCACCCTACTGTTTGTTGAAATTCTTAGAGAAAGAGATGTCCAAGCTCAATGGTTTGATGTAAGAAAAGTTA | |
| TGAGGACAAATGATAGATTCGGACGTGCAGAACCTGATATTGCCGCTCTCGCTGAACTTGCCGCATTACAATTACT | |
| CCCCCGACTAAATGAAGGGCTTGTTATTACACAAGGTTTTATAGGGTCAGAAAACAAAGGTCGAACAACAACTCTT | |
| GGTAGAGGAGGTTCCGATTACACTGCTGCACTACTTGCCGAAGCTCTACATGCCTCAAGAGTTGATATATGGACAG | |
| ACGTGCCAGGAATTTATACTACTGATCCTAGGGTTGTTAGCGCCGCAAAAAGAATCGACGAAATTGCATTCGCAGA | |
| GGCTGCAGAAATGGCTACATTTGGAGCTAAAGTGTTACATCCAGCTACTCTACTACCCGCAGTTAGGTCCGACATT | |
| CCAGTGTTCGTTGGAAGTTCAAAAGATCCAAGAGCAGGCGGGACACTTGTATGTAATAAAACCGAAAATCCACCTT | |
| TATTCAGAGCACTTGCCCTTAGACGTAACCAAACACTGTTGACTCTCCATTCTTTAAACATGCTACATTCCCGAGG | |
| TTTCCTTGCAGAAGTTTTCGGTATCCTTGCTAGACATAATATTTCAGTAGATCTTATAACCACAAGCGAAGTTTCA | |
| GTTGCCCTCATATTAGACACTACAGGTTCCACATCAACCGGAGATACTTTACTAACACAATCACTTTTAATGGAAC | |
| TATCAGCACTCTGTCGAGTTGAAGTCGAAGAAGGACTAGCATTAGTAGCCCTCATCGGAAATGACCTTAGCAAAGC | |
| TTGTGCAGTGGGGAAGGAAGTTTTTGGAGTCTTGGAGCCTTTTAATATAAGAATGATATGCTACGGCGCCTCTTCA | |
| CACAATCTTTGTTTTTTAGTGCCTGGAGAAGATGCAGAACAAGTTGTACAGAAACTTCATTCTAATCTCTTTGAAT | |
| GAGAATTC. | |
| Aspartate kinase gene from Streptomyces | |
| SEQ ID 17 | |
| ATGGGCCTTGTCGTGCAGAAGTACGGCGGCTCATCCGTTGCGGATGCCGAGGGCATCAAGCGCGTTGCCAAGCGAA | |
| TCGTCGAGGCCAAGAAGAACGGCAACCAGGTTGTCGTCGTGGTCTCGGCGATGGGCGACACGACGGACGAGCTGAT | |
| CGATCTCGCGCAGGAAGTGTCCCCGATCCCGTCGGGACGCGAGTTCGACATGCTGCTGACCGCCGGAGAGCGGATC | |
| TCCATGGCCCTGCTGGCGATGGCGATCAAAAACCTCGGCCATGAGGCGCAGTCCTTCACCGGTAGCCAGGCCGGTG | |
| TGATCACTGACTCCGTGCACAACAAGGCACGGATCATCGATGTCACGCCGGGCCGCATCAAGGCGTCCCTCGACGA | |
| GGGCAACATCGCCATCGTCGCCGGATTCCAGGGCGTGTCCCAGGACAAGAAGGACATCACGACGCTCGGGCGCGGT | |
| GGTTCGGACACCACCGCGGTGGCGCTCGCCGCCGCCCTGAACGCCGATGTCTGCGAGATCTACACCGACGTCGACG | |
| GCGTCTTCACCGCCGACCCGCGGGTCGTGAAGAAGGCCCGGAAGATCGAGTGGATCTCCTTCGAGGACATGCTGGA | |
| GCTGGCCAGCTCCGGCTCCAAGGTGCTGCTGCACCGCTGCGTCGAGTACGCACGCCGTTACAACATCCCGATCCAC | |
| GTCCGCTCCTCCTTCTCGGGGCTGCAGGGCACATGGGTCAGCAACGAACCGCAAGGGGACAGGCCGATGGAACAGG | |
| CGATCATCTCGGGCGTCGCACACGACACCTCCGAGGCGAAGGTCACGGTCGTCGGGGTCCCGGACAAGCCGGGCGA | |
| GGCCGCGCGGATCTTCCGTGCCATCGCCGACTCCGAGGTCAACATCGACATGGTCGTCCAGAACGTGTCGGCGGCC | |
| TCCACCGGTCTGACGGACATCTCCTTCACGCTGCCGAAGGCCGAGGGCCGCAAGGCCGTCGCCGCGCTGGAGAAGA | |
| CCCGGGCCGCGGTCGGCTTCGACTCGCTCCGCTACGACGACCAGATCGCCAAGATCTCGCTGGTCGGCGCGGGCAT | |
| GAAGACCAACCCCGGCGTCACCGCGACGTTCTTCGAGGCGCTGTCGAACGCGGGCGTGAACATCGAGCTCATCTCG | |
| ACCTCCGAGATCCGCATCTCGGTCGTCACCCGTGCCGATGACGTCAACGAGGCCGTCCAGGCGGTGCACAGCGCCT | |
| TCGGCCTCGACAGTGAGACCGACGAAGCAGTCGTCTACGGCGGCACCGGGCGATGA. | |
| Codon-optimized aspartate kinase gene from Streptomyces | |
| SEQ ID 18 | |
| ATGGGACTTGTTGTACAAAAATATGGAGGAAGTTCAGTTGCCGACGCCGAGGGAATAAAACGTGTAGCAAAGCGAA | |
| TTGTCGAAGCAAAAAAAAACGGAAATCAAGTAGTTGTAGTTGTATCAGCAATGGGAGATACTACCGATGAACTTAT | |
| TGATCTAGCACAGGAAGTTTCACCAATTCCTAGCGGTAGAGAATTTGATATGCTTTTGACTGCAGGCGAACGTATA | |
| AGCATGGCATTACTAGCAATGGCTATTAAAAATCTAGGACATGAAGCTCAGTCATTTACAGGCTCTCAAGCAGGCG | |
| TTATAACCGATTCTGTTCATAATAAAGCTCGAATTATAGATGTTACACCAGGTCGAATCAAAGCAAGTTTAGATGA | |
| AGGTAATATAGCTATAGTGGCCGGATTTCAAGGAGTTTCACAAGACAAGAAGGACATTACTACTCTAGGTAGGGGC | |
| GGATCTGATACTACTGCTGTTGCACTTGCTGCTGCCTTAAATGCTGATGTTTGCGAAATATACACAGATGTAGATG | |
| GCGTTTTTACTGCTGACCCAAGAGTTGTTAAAAAAGCAAGAAAAATAGAATGGATCTCATTTGAAGATATGTTGGA | |
| GCTCGCTTCCTCAGGTAGTAAAGTACTGTTACATCGTTGCGTCGAATATGCTCGAAGATATAATATACCAATCCAC | |
| GTTCGATCCAGCTTTTCAGGACTTCAAGGTACATGGGTTTCAAACGAGCCACAAGGTGATAGACCAATGGAACAAG | |
| CAATTATTAGTGGTGTTGCACATGATACTTCCGAAGCTAAAGTTACCGTAGTTGGCGTACCAGATAAACCAGGAGA | |
| AGCTGCAAGAATTTTTAGAGCTATAGCAGATTCAGAAGTTAATATAGATATGGTTGTTCAGAATGTTAGTGCTGCT | |
| TCAACAGGATTGACTGATATTTCTTTCACTTTACCTAAAGCTGAAGGCAGAAAAGCCGTCGCTGCCCTTGAAAAAA | |
| CACGAGCTGCTGTTGGATTCGATTCCTTAAGATATGATGATCAAATTGCAAAGATTAGTTTAGTTGGAGCTGGAAT | |
| GAAAACAAACCCAGGCGTTACTGCAACATTTTTTGAAGCCCTCTCAAATGCTGGTGTTAATATTGAGCTCATTTCA | |
| ACTTCAGAGATTAGAATTTCAGTAGTTACTAGAGCTGATGACGTTAATGAAGCTGTTCAAGCTGTTCACAGTGCAT | |
| TCGGATTGGATTCTGAGACTGACGAAGCTGTTGTGTATGGTGGTACTGGTAGA. | |
| Promoter of the Fmo gene | |
| SEQ ID 19 | |
| CCTTAATTCTATACACTATTATTTCCTCTTTTATTCTACATTTCATTCTTAGCTTATTTTTTCGTAAACGTTACCC | |
| AGTGCCACTACCACCCGGTCCAAAACCATGGCCAATAATCGGAAACATAGTCCAATTAGGTCCGAAGCCGCACCAG | |
| TCCACTGCATCAATGGCCCGAACTTACGGGCCACTCATGCACCTTCGCATGGGGTTCGTGGACGTGGTGGTTGCGG | |
| CCTCAGCTTCGGTGGCGGCTCAATTTTTGAAAAATCATGACGCTAACTTCTCGAGCCGCCCACCGAACTCTGGGGC | |
| GAAACACATGGCTTATAATTACCATGACCTTGTGTTTGCACCTTACGGACCACGGTGGCGTATGCTAAGGAAAATT | |
| TGTTCTGTTCATCTCTTTTCGGCTAAAGCTTTAGATGACTTCCGCCATGTCCGACAGGAAGAAGTCAGAACACTTA | |
| CGCGCGCCTTAGCAAATGCTGGCCAAAAGCCAATCAAATTAGGGCAGCTGTTGAACGTGTGCACCACGAATGCACT | |
| TGCGCGTGTGATGCTCGGGAAGCGGGTATTCGCCGACGGTACTAACGGTATCGATCCACAAGCGGAGGAGTTCAAG | |
| TTAATGGTGGTGGAGATGATGGTGCTCGCCGGCGTTTTCACATCGGCGATTTTATTCCGGCGCTTGATTGGATGGA | |
| CATTCAAGGCGTAGCAGGAAAAATGAAGAAACTCCACGCGCGTTTCGACGCGTTCTTAACCACGATCCTCGAAGAA | |
| CACAAGGGAAAGCGAGTTGGAGAATCGAAGGAGCAGGGGGATTTGTTGAATACGTTGATCTCTCTGAAAAATGAAG | |
| AAGACGATAATGGAGGAAAGCTTACTGATACAGAAATTAAAGCTTTACTTTGGGTACGCCTCTTACAATTATCTCT | |
| TTATTTCAAATTGGACAAGTAAAAACAAATATGGATTTTTAGTATATCTAACAAGTAAAAAGGAATAGAGGTAATA | |
| AATATGAAACTATGCCATTTTTCTTTGACGGACTAAAAATGGAAGTATGCTAATGTCCTAATTTATATGATAATGT | |
| TTGGCTTGAAACAATGTTGTTTAAGAAGTTAATTTTTATTCGTCCTGCAATTTTAATGGTATGAGTTCGAATTTCA | |
| GGATATAGTTTGATCAATTGTTCTTATACAAATTCACTCTAATATTACAAACTTACAAATTTGAAGTTTAAAGATT | |
| TATCAGTTCAAATTTCATGATTTTTCACCTTTTCAAAGCCTTAAACTCGAATTATACAAGTGTGGGAGTTATT. | |
| Glutenin gene | |
| SEQ ID 20 | |
| ATGGCTAAGCGGTTAGTCCTCTTTGTGGCGGTAGTCGTCGCCCTCGTGGCTCTCACCGTCGCTGAAGGTGAGGCCT | |
| CTGAGCAACTACAGTGTGAGCGCGAGCTCCAGGAGCTCCAGGAGCGCGAGCTCAAGGCATGCCAGCAGGTCATGGA | |
| CCAGCAGCTCCGAGACATTAGCCCCGAGTGCCACCCCGTCGTCGTCAGCCCGGTCGCGGGACAATACGAGCAGCAA | |
| ATCGTGGTGCCCAAGGGCGGATCTTTCTACCCCGGCGAGACCACGCCACCGCAGCAACTCCAACAACGTATATTTT | |
| GGGGAATACCTGCACTACTAAAAAGGTATTACCCAAGTGTAACTTCTCCGCAGCAGGTTTCATACTATCCAGGCCA | |
| AGCTTCTCCGCAACGGCCAGGACAAGGTCAGCAGCCAGGACAAGGGCAACAATCAGGACAAGGACAACAAGGGTAC | |
| TACCCAACTTCTCCGCAACAGCCAGGACAATGGCAACAACCGGAACAAGGGCAACCAGGGTACTACCCAACTTCTC | |
| CGCAGCAGCCAGGACAATTGCAACAACCAGCACAAGGGCAGCAACCAGGACAAGGACAACAAGGTCGGCAGCCAGG | |
| ACAAGGGCAACCAGGGTACTACCCAACTTCTTCGCAGCTGCAGCCAGGACAATTGCAACAACCAGCACAAGGGCAA | |
| CAAGGGCAGCAACCAGGACAAGGGCAACAAGGTCAACAGCCAGGACAAGGGCAACAACCAGGACAAGGACAACAAG | |
| GTCAACAGCCAGGACAAGGGCAACAACCAGGACAAGGGCAACAAGGTCAGCAGCTCGGACAAGGACAACAAGGGTA | |
| CTACCCAACTTCTCTGCAACAGTCGGGACAAGGGCAACCAGGGTACTACCCAACTTCTCTGCAGCAGCTAGGACAA | |
| GGGCAATCAGGGTACTACCCAACTTCTCCGCAGCAACCAGGACAAGGGCAGCAGCCAGGACAATTGCAACAACCAG | |
| CACAAGGGCAGCAACCAGAACAAGGGCAACAAGGTCAGCAGCCAGGACAAGGGCAACAAGGCCAGCAGCCAGGACA | |
| AGGGCAGCAACCGGGACAAGGGCAACCAGGGTACTACCCAACTTCTCCGCAGCAGTCAGGACAAGGGCAACCAGGG | |
| TACTACCCAACTTCTTCGCAGCAGCCAACACAATCGCAGCAACCAGGACAAGGGCAACAAGGTCAGCAGGTAGGAC | |
| AAGGGCAACAAGCTCAGCAGCCAGGACAAGGGCAGCAACCGGGACAAGGGCAGCCAGGGTACTACCCAACTTCTCC | |
| GCTGCAGTCAGGACAAGGGCAACCAGGGTACTACCTAACTTCTCCGCAGCAGTCAGGACAAGGGCAGCAGCCAGGA | |
| CAATTGCAACAATCAGCACAAGGGCAAAAAGGACAGCAACCAGGACAAGGTCAACAGCCAGGGCAAGGGCAACAAG | |
| GTCAGCAGCCAGGACAAGGGCAACAAGGTCAGCAACCGGGGCAAGGGCAGCCAGGGTACTACCCAACTTCTCCGCA | |
| GCAATCAGGACAAGGGCAACAGCCAGGACAATGGCAACAACCAGGACAAGGGCAACCAGGATACTACCCAACTTCT | |
| CCGTTGCAGCCAGGACAAGGGCAACCAGGGTACGACCCAACTTCTCCGCAACAGCCAGGACAAGGGCAGCAACGAG | |
| GACAATTGCAACAACCAGCACAAGGGCAACAAGGGCAGCAACTAGCACAAGGGCAACAAGGGCAGCAACCAGCACA | |
| AGTGCAACAAGGGCAGCAGCCAGCACAAGGGCAACAAGGTCAGCAGCTAGGACAAGGGCAACAAGGTCAGCAGCCA | |
| GGACAAGGGCAGCAACCAGCACAAGGGCAACAAGGTCAGCAGCCAGGACAAGGGCAACAAGGTCAGCAGCCAGGAC | |
| AAGGGCAGCAACCGGGACAAGGGCAGCCATGGTACTACCCAACTTCTCCGCAGGAGTCAGGACAAGGGCAACAGCC | |
| AGGACAATGGCAACAACCAGGACAATGGCAACAACCAGGACAAGGGCAACCAGGGTACTACCTAACTTCTCCGTTG | |
| CAGCTAGGACAAGGGCAACAAGGGTACTACCCAACTTCTCTGCAACAACCAGGACAAGGGCAGCAACCAGGACAAT | |
| GGCAACAATCGGGACAAGGGCAACATGGGTACTACCCAACTTCTCCGCAGCTGTCAGGACAAGGGCAACGGCCAGG | |
| ACAATGGCTGCAACCAGGACAAGGGCAACAAGGGTACTACCCAACTTCTCCGCAACAGTCAGGACAAGGGCAACAA | |
| CTAGGACAATGGCTGCAACCAGGACAAGGGCAACAAGGGTACTACCCAACTTCTCTGCAACAGACAGGACAAGGGC | |
| AGCAATCAGGACAAGGGCAACAAGGCTACTACAGCTCATACCATGTTAGCGTGGAGCACCAGGCGGCCAGCCTAAA | |
| GGTGGCAAAGGCGCAGCAGCTCGCGGCACAGCTGCCGGCAATGTGCCGGCTGGAGGGCGGCGACGCATTGTCGGCC | |
| AGCCAGTGA. | |
| Ppo gene fragment | |
| | SEQ ID 21 |
| TTAGTCTCTATTGAATCTGCTGAGATTACACTTTGATGGATGATGCTCTGTTTTTGTTTTCTTGTTCTGTTTTTTC | |
| CTCTGTTGAAATCAGCTTTGTTGCTTGATTTCATTGAAGTTGTTATTCAAGAATAAATCAGTTACAATTATGTTTG | |
| GG. | |
| R1 gene fragment | |
| SEQ ID 22 | |
| ACCTTATTTCACTACCACTTTCCACTCTCCAATCCCCATACTCTCTGCTCCAATCTTCATTTTGCTTCGTGAATTC | |
| ATCTTCATCGAATTTCTCGACGCTTCTTCGCTAATTTCCTCGTTACTTCACTAAAAATCGACGTTTCTAGCTGAAC | |
| TTGAGTGAATTAAGCCAGTGGGAGGAT. | |
| PhL gene fragment | |
| SEQ ID 23 | |
| TTAGAGTGTGGGTAAGTAATTAAGTTAGGGATTTGTGGGAAATGGACAAATATAAGAGAGTGCAGGGGAGTAGTGC | |
| AGGAGATTTTCGTGCTTTTATTGATAAATAAAAAAAGGGTGACATTTAATTTCCACAAGAGGACGCAACACAACAC | |
| ACTTAATTCCTGTGTGTGAATCAATAATTGACTTCTCCAATCTTCATCAATAAAATAATTCACAATCCTCACTCTC | |
| TTATCACTCTCATTCGAAAAGCTAGATTTGCATAGAGAGCACAAA. | |
| Gs2 gene | |
| SEQ ID 24 | |
| GTTCATCTTCTTCTCTCACTTCTCTTAACAACATCTTTTCTGCATTGGCCACTTAGTTGGTTAGGAGGTGAACATG | |
| GCTCAGATTCTGGCTCCATCTGCACAATGGCAGATGAGAATGACAAAGAGCTCAACAGATGCTAGTCCCTTGACTT | |
| CAAAGATGTGGAGCTCTGTGGTGCTGAAGCAGAACAAAAGACTTGCTGTTAAAAGCTCTGCCAAATTTAGAGTTTT | |
| TGCCCTCCAGTCTGACAATGGCACCGTGAACAGAATGGAACAGCTGCTAAACTTGGACGTAACTCCATACACTGAT | |
| AAGATCATTGCTGAATATATTTGGATCGGGGGGACTGGAATTGATGTGCGCAGTAAATCAAGGACTATTTCAAAAC | |
| CAGTCAAGGATGCTTCTGAGCTCCCAAAGTGGAACTACGATGGATCAAGTACTGGACAAGCACCTGGAGAAGACAG | |
| TGAAGTCATTCTATATCCTCAGGCAATATTCAAAGACCCTTTCCGTGGTGGTAACAACATCTTGGTTATCTGTGAT | |
| ACCTACACACCAGCTGGAGAGCCAATTCCTACAAACAAACGCCATAAAGCTGCTCAAATTTTTAGCGACCCAAAAG | |
| TTGCATCTCAAGTTCCATGGTTTGGAATAGAACAAGAGTACACCTTACTCCAGCCAAATGTTAACTGGCCCTTAGG | |
| TTGGCCTGTTGGAGGCTATCCTGGCCCACTAGGTTCTTACTACTGTGGTGCTGGAGTGGAAAAGTCATTTGGCCGA | |
| GATATATCAGATGCTCACTACAAGGCTTGCCTGTATGCTGGAATTAACATTAGTGGTACTAACGGAGAGGTTATGC | |
| CAGGACAGTGGGAATTTCAAGTAGGACCTAGTGTTGGAATTGAAGGTGGAGATCATATCTGGTGTGCTAGATACCT | |
| CCTCGAGAGAATTACTGAACAAGCAGGAGTTGTCCTCTCACTCGATCCAAAACCAATTGAGGGTGACTGGAACGGT | |
| GCAGGATGCCACACTAACTACAGTACACTGAGTATTAGAGAAGAGGGAGGCTTTGAAGTGATAAAGAAAGCAATTC | |
| TTAATCTATCCCTGCGCCACAAGGGACATATAAGTGCTTATGGAGAAGGAAATGAGAGAAGGTTGACCGGAAAGCA | |
| TGAAACTGCTAGTATTGACCAATTTTCATGGGGAGTTGCTAACCGTGGTTGCTCAATCCGTGTGGGGCGTTACACT | |
| GAAAAAGAAGCCAGGGGTTATTTGGAAGACCGCCGCCCAGCTTCAAACATGGACCCCTATGTTGTGACCGCATTAC | |
| TTGCCGAAACTACTATACTGTGGGAGCCAACCCTTGAGGCTGAAGCTCTTGCTGCCCAAAAGATCTCATTGAAGGT | |
| TTAGAGTAATTGAGGGGAAATGTTTTCATCATAATCCTTCTTAGAATTTATGAGATAAGTGTGAAGCTTGTACCTT | |
| GTTGAGATTCCCTTATTTGGGAAATTCTTGTAAAGGAATTCAAAATTTAACCAGTTCATCCTAGAAAGAGGTTCCT | |
| TAAGACATGAGACTACTTTGGAGTTGAGGTGTAATTGTTGGACTACTTTGAACATCTTTACCTTTCTTTTCTCCAG | |
| ATGAATCCATTTCTCTGAAATTCCAATTGGTGTGATTTTTCCGAAAAAAAAAAAAAAAAAAAA. | |
| Gs1A gene | |
| SEQ ID 25 | |
| GATCAAATTAATTAAATTCTCTCATTCAACAAATTCAAACTTTAGAATTATTATTATTTTCACATAATGGCTCATC | |
| TTTCAGATCTTGTCAATCTCAATCTCTCTGATTCCTCTGACAAAATCATTGCTGAATACATATGGATTGGTGGATC | |
| AGGAATGGATGTAAGGAGCAAAGCCAGGACTCTATCTGGTCCTGTTGATGATCCTTCAAAGCTTCCCAAATGGAAT | |
| TATGATGGTTCTAGCACAGGTCAAGCTCCTGGAGAAGACAGTGAAGTGATCCTATATCCTCAAGCAATTTTCAAGG | |
| ATCCATTCAGGAGGGGCAACAATATCCTGGTCATCTGTGATTGTTACACCCCAGCTGGTGAACCAATTCCAACAAA | |
| CAAGATGTGTCACAATGCTGCTAAAATATTTAGTAACCCTGATGTTGTTGTTGAGGAACCATGGTATGGTCTTGAA | |
| GCAGAATACACCTTGCTACAAAGAAGGAATTACTGGCCTCTTGGATGGCCTATTGGTGGTTTTCCTGGATTACAGG | |
| GACCATACTACTGTGGAATTGGATCTGGAAAGGCTTTTGGACGCGATATTGTTGATGCTCATTACAAGGCATATAT | |
| CTATGCCGGGATTAACATTAGTGGTATCAACGGAGAAGTGATGCCTGGACAGTGGGAATTTCAAGTTGGTCTTCAA | |
| GTTGGCATTGCATCAGGTGACGAAGTGTGGGCAGCTCGTTACATTCTCGAGAGGATTACAGAGATTGCTGGAGTTG | |
| TCGTGTCATTCGACCCCAAACCTATTCCGGGCGACTGGAATGGTGCAGGAGCGCATACAAATTACAGTACCAAGTC | |
| CATGAGGAATGAGGGAGGGTATGAAGTTATCAAGAAGGCTATTGAGAAGCTTGGACTTAGGCACAAGGAGCACATT | |
| GCAGCATATGGTGAAGGCAATGAACGTCGTCTCACTGGAAAGCACGAAACAGCTGACATCAACACGTTCAAATGGG | |
| GTGTTGCAAATCGTGGTGCATCCATTCGTGTGGGAAGAGACACGGAGAAGGAAGGCAAGGGATACTTTGAGGACAG | |
| GAGGCCTGCATCGAACATGGATCCATACATCGTGACCTCTATGTACGCGGAGACTACCCTCCTGTGGAACCCTTGA | |
| ACGCGTATGGGATGAATATTCTCGGGGGTGCAAACATATGAGGAAAGAATTGAATTTCTTAACAGGCCCTTTCCTC | |
| ACATGTCCTTAAGAGAGTTATGTAGCTAGTAATTTTGATATATTATGTTGTTTTCTAAGTTTCAATTTGTATTGTA | |
| CTCAGCAAGCCTGAGTTCATTGGCCAAAATCGATTTGGCAATGTTGTTAAAAATAAGAGTTTTAATCTTATTAATA | |
| ACAATATGGAAGGGTTTAACTTAAAAAAAAAA. | |
| Gs1B gene | |
| SEQ ID 26 | |
| GATCTTATAGAGAATTTCAATTTTAAGAAGTTATCATCATGTCTCTGCTTTCAGATCTTATCAACCTCAATCTCTC | |
| AGATGATACTCAGAAGATCATTGCTGAATACATATGGATTGGTGGATCAGGCATGGACATGAGGAGCAAAGCCAGG | |
| ACTCTCCCTGGTCCAGTTACTAGTCCTGCAGAACTACCCAAATGGAACTATGATGGATCAAGCACAGGTCAAGCTC | |
| CTGGAGAAGACAGTGAAGTGATCATATACCCACAAGCAATCTTCAAGGATCCATTCAGGAGAGGCAACAATATCTT | |
| GGTCATGTGTGATGCCTATACTCCTGCTGGTGAGCCCATCCCAACAAACAAGAGGCACGCCGCTGCCAAGGTCTTC | |
| TGCCACCCTGATGTGGCTGCTGAGGAAACTTGGTATGGTATTGAACAAGAATATACCTTGCTGCAAAAGGAGGTCA | |
| ACTGGCCTCTTGGATGGCCCATTGGCGGTTTTCCTGGACCCCAGGGACCATACTACTGTGGAACTGGAGCTGACAA | |
| GGCCTTTGGACGTGACATTGTGGACGCCCATTACAAGGCATGTCTCTATGCTGGGATTAATATCAGCGGAATCAAT | |
| GGTGAAGTCATGCCGGGACAGTGGGAATTCCAAGTGGGACCTTCTGTTGGCATCTCAGCCGGTGATGAAGTGTGGG | |
| TAGCTCGTTACATTCTAGAGAGGATTGCAGAGATTGCTGGGGTGGTCGTGTCATTCGACCCCAAGCCTATTCCGGG | |
| CGACTGGAACGGCGCAGGTGCTCACACAAATTACAGCACCAAGTCGATGAGGGAAGACGGAGGCTATAAAATAATC | |
| TTGAAGGCTATTGAGAAGCTTGGCCTGAAGCACAAAGAACACATTGCTGCATATGGTGAAGGCAATGAGCGTCGTC | |
| TCACTGGAAAGCACGAAACAGCCAACATCAACACCTTCAAATGGGGGGTTGCAAACCGTGGTGCATCTGTCCGTGT | |
| TGGAAGAGACACAGAGAAGGCAGGCAAGGGATACTTTGAGGACAGAAAATATGGACCCATACGTCGTTACCTCCAT | |
| GATCGCAGAAACCACCATCATCGGTTAACCTTGAAGACATTTTACTATGGATGGCTCGGGGGATCGCTTGTTTCTG | |
| GTTTGCACAATTTGGGATAGGAGAAAAGATTGAATTGTGAAACGACCCTTTCGACTTCACCTGTGTTAATTTTTAG | |
| TTATAGGGGTAGATTGTCTCTTGTTATTTTTCTGTTTATTTGCCAGTTGAATTGTATTTTCATACAGCAAGGCCTT | |
| ATACATTGTCTATGATTTGGCAATGCTGTGTTACAAAACAATGTTATTCTTATTAATAACAAAGATAATGAAAGGA | |
| TTTGATTCTATTGCTCAAAAAAAAAAAAA. | |
| Antisense segment comprising fragments of the Gs1B and Gs2 genes | |
| SEQ ID 27 | |
| GTTTGTAGGAATTGGCTCTCCAGCTGGTGTGTAGGTATCACAGATAACCAAGATGTTGTTACCACCACGGAAAGGG | |
| TCTTTGAATATTGCCTGAGGATATAGAATGACTTCACTGTCTTCTCCAGGTGCTTGTCCAGTACTTGATCCATCGT | |
| AGTTCCACTTTGGGAGCTCAGAAGCATCCTTGACTGGTTTTGAAATAGTCCTTGATTTACTGCGCACATCAATTCC | |
| AGTCCCCCCGATCCAAATATATTCAGCAATGATCTTATCAGTGTATGGAGTTACGTCCAAGTTTAGCAGCTGTTCC | |
| ATTCTGTTCACGGTGCCATTGTCAGACTGGAGGGCAAAAACTCTAAATTTGGCAGAGCTTTTAACAGCAAGTCTTT | |
| TGTTCTGCTTCAGCACCACAGAGCTCCACATCTTTGAAGTCAAGGGACTAGCATCTGTTGAGCTCTTTGTCATTCT | |
| CATCTGCCATTGTGCAGATGGAGCCAGAATCTGAGCCATCTGTCCTCAAAGTATCCCTTGCCTGCCTTCTCTGTGT | |
| CTCTTCCAACACGGACAGATGCACCACGGTTTGCAACCCCCCATTTGAAGGTGTTGATGTTGGCTGTTTCGTGCTT | |
| TCCAGTGAGACGACGCTCATTGCCTTCACCATATGCAGCAATGTGTTCTTTGTGCTTCAGGCCAAGCTTCTCAATA | |
| GCCTTCAAGATTATTTTATAGCCTCCGTCTTCCCTCATCGACTTGGTGCTGTAATTTGTGTGAGCACCTGCGCCGT | |
| TCCAGTCGCCCGGAATAGGCTTGGGGTCGAATGACACGACCACCCCAGCAATCTCTGCAATCCTCTCTAGAATGTA | |
| ACGAGCTACCCACACTTCATCACCGGCTGAGATGCCAACA. | |
| Sense segment comprising fragments of the Gs1B and Gs2 genes | |
| SEQ ID 28 | |
| GCCACCCTGATGTGGCTGCTGAGGAAACTTGGTATGGTATTGAACAAGAATATACCTTGCTGCAAAAGGAGGTCAA | |
| CTGGCCTCTTGGATGGCCCATTGGCGGTTTTCCTGGACCCCAGGGACCATACTACTGTGGAACTGGAGCTGACAAG | |
| GCCTTTGGACGTGACATTGTGGACGCCCATTACAAGGCATGTCTCTATGCTGGGATTAATATCAGCGGAATCAATG | |
| GTGAAGTCATGCCGGGACAGTGGGAATTCCAAGTGGGACCTTCTGTTGGCATCTCAGCCGGTGATGAAGTGTGGGT | |
| AGCTCGTTACATTCTAGAGAGGATTGCAGAGATTGCTGGGGTGGTCGTGTCATTCGACCCCAAGCCTATTCCGGGC | |
| GACTGGAACGGCGCAGGTGCTCACACAAATTACAGCACCAAGTCGATGAGGGAAGACGGAGGCTATAAAATAATCT | |
| TGAAGGCTATTGAGAAGCTTGGCCTGAAGCACAAAGAACACATTGCTGCATATGGTGAAGGCAATGAGCGTCGTCT | |
| CACTGGAAAGCACGAAACAGCCAACATCAACACCTTCAAATGGGGGGTTGCAAACCGTGGTGCATCTGTCCGTGTT | |
| GGAAGAGACACAGAGAAGGCAGGCAAGGGATACTTTGAGGACAGAATGGCTCAGATTCTGGCTCCATCTGCACAAT | |
| GGCAGATGAGAATGACAAAGAGCTCAACAGATGCTAGTCCCTTGACTTCAAAGATGTGGAGCTCTGTGGTGCTGAA | |
| GCAGAACAAAAGACTTGCTGTTAAAAGCTCTGCCAAATTTAGAGTTTTTGCCCTCCAGTCTGACAATGGCACCGTG | |
| AACAGAATGGAACAGCTGCTAAACTTGGACGTAACTCCATACACTGATAAGATCATTGCTGAATATATTTGGATCG | |
| GGGGGACTGGAATTGATGTGCGCAGTAAATCAAGGACTATTTCAAAACCAGTCAAGGATGCTTCTGAGCTCCCAAA | |
| GTGGAACTACGATGGATCAAGTACTGGACAAGCACCTGGAGAAGACAGTGAAGTCATTCTATATCCTCAGGCAATA | |
| TTCAAAGACCCTTTCCGTGGTGGTAACAACATCTTGGTTATCTGTGATACCTACACACCAGCTGGAGAGCCAATTC | |
| CTACAAAC. | |
| DHPS gene | |
| SEQ ID 29 | |
| CCAGGCGACTGTCTTCAATATTACAGCCGCAACTACTGACATGACGGGTGATGGTGTTCACAATTCCACGGCGATC | |
| GGCACCCAACGCAGTGATCACCAGATAATGTGTTGCGATGACAGTGTCAAACTGGTTATTCCTTTAAGGGGTGAGT | |
| TGTTCTTAAGGAAAGCATAAAAAAAACATGCATACAACAATCAGAACGGTTCTGTCTGCTTGCTTTTAATGCCATA | |
| CCAAACGTACCATTGAGACACTTGTTTGCACAGAGGATGGCCCATGTTCACGGGAAGTATTGTCGCGATTGTTACT | |
| CCGATGGATGAAAAAGGTAATGTCTGTCGGGCTAGCTTGAAAAAACTGATTGATTATCATGTCGCCAGCGGTACTT | |
| CGGCGATCGTTTCTGTTGGCACCACTGGCGAGTCCGCTACCTTAAATCATGACGAACATGCTGATGTGGTGATGAT | |
| GACGCTGGATCTGGCTGATGGGCGCATTCCGGTAATTGCCGGGACCGGCGCTAACGCTACTGCGGAAGCCATTAGC | |
| CTGACGCAGCGCTTCAATGACAGTGGTATCGTCGGCTGCCTGACGGTAACCCCTTACTACAATCGTCCGTCGCAAG | |
| AAGGTTTGTATCAGCATTTCAAAGCCATCGCTGAGCATACTGACCTGCCGCAAATTCTGTATAATGTGCCGTCCCG | |
| TACTGGCTGCGATCTGCTCCCGGAAACGGTGGGCCGTCTGGCGAAAGTAAAAAATATTATCGGAATCAAAGAGGCA | |
| ACAGGGAACTTAACGCGTGTAAACCAGATCAAAGAGCTGGTTTCAGATGATTTTGTTCTGCTGAGCGGCGATGATG | |
| CGAGCGCGCTGGACTTCATGCAATTGGGCGGTCATGGGGTTATTTCCGTTACGACTAACGTCGCAGCGCGTGATAT | |
| GGCCCAGATGTGCAAACTGGCAGCAGAAGAACATTTTGCCGAGGCACGCGTTATTAATCAGCGTCTGATGCCATTA | |
| CACAACAAACTATTTGTCGAACCCAATCCAATCCCGGTGAAATGGGCATGTAAGGAACTGGGTCTTGTGGCGACCG | |
| ATACGCTGCGCCTGCCAATGACACCAATCACCGACAGTGGTCGTGAGACGGTCAGAGCGGCGCTTAAGCATGCCGG | |
| TTTGCTGTAAAGTTTAGGGAGATTTGATGGCTTACTCTGTTCAAAAGTCGCGCCTGG. | |
| Cg DHPS gene | |
| SEQ ID 30 | |
| ATGAGCACAGGTTTAACAGCTAAGACCGGAGTAGAGCACTTCGGCACCGTTGGAGTAGCAATGGTTACTCCATTCA | |
| CGGAATCCGGAGACATCGATATCGCTGCTGGCCGCGAAGTCGCGGCTTATTTGGTTGATAAGGGCTTGGATTCTTT | |
| GGTTCTCGCGGGCACCACTGGTGAATCCCCAACGACAACCGCCGCTGAAAAACTAGAACTGCTCAAGGCCGTTCGT | |
| GAGGAAGTTGGGGATCGGGCGAAGCTCATCGCCGGTGTCGGAACCAACAACACGCGGACATCTGTGGAACTTGCGG | |
| AAGCTGCTGCTTCTGCTGGCGCAGACGGCCTTTTAGTTGTAACTCCTTATTACTCCAAGCCGAGCCAAGAGGGATT | |
| GCTGGCGCACTTCGGTGCAATTGCTGCAGCAACAGAGGTTCCAATTTGTCTCTATGACATTCCTGGTCGGTCAGGT | |
| ATTCCAATTGAGTCTGATACCATGAGACGCCTGAGTGAATTACCTACGATTTTGGCGGTCAAGGACGCCAAGGGTG | |
| ACCTCGTTGCAGCCACGTCATTGATCAAAGAAACGGGACTTGCCTGGTATTCAGGCGATGACCCACTAAACCTTGT | |
| TTGGCTTGCTTTGGGCGGATCAGGTTTCATTTCCGTAATTGGACATGCAGCCCCCACAGCATTACGTGAGTTGTAC | |
| ACAAGCTTCGAGGAAGGCGACCTCGTCCGTGCGCGGGAAATCAACGCCAAACTATCACCGCTGGTAGCTGCCCAAG | |
| GTCGCTTGGGTGGAGTCAGCTTGGCAAAAGCTGCTTCGCGTCTGCAGGGCATCAACGTAGGAGATCCTCGACTTCC | |
| AATTATGGCTCCAAATGAGCAGGAACTTGAGGCTCTCCGAGAAGACATGAAAAAAGCTGGAGTTCTA. | |
| Amino acid-substituted DHPS gene | |
| SEQ ID 31 | |
| ATGATTTCGCCGACGAATCTCCTCCCGGCGCGGAAGATCACCCCTGTCTCAAATGGCGGCGCAGCGACGGCGAGCC | |
| CCTCTTCTCCCTCGGTGGCCGCACGGCCACGGCGACTCCCTTCAGGCCTCCAATCTGTGACTGGTAGAGGGAAGGT | |
| TTCCTTGGCAGCCATCACTCTAGATGATTACCTTCCAATGCGAAGCACTGAAGTGAAGAACCGGACATCAACAGAT | |
| GACATCACAAGGCTGAGACTAATCACAGCAGTTAAAACCCCCTATTTGCCAGATGGGAGGTTCGATCTGGAAGCAT | |
| ATGATTCTCTCATAAACATGCAGATAGAGGGTGGTGCCGAAGGCGTAATAGTTGGAGGAACAACAGGAGAGGGTCA | |
| CCTCATGAGCTGGGACGAACATATCATGCTCATTGGGCACACAGTGAACTGCTTTGGCTCTAGAATCAAAGTGATA | |
| GGCAACACAGGAAACAACTCAACCAGAGAAGCCGTCCACGCAACAGAACAGGGATTTGCTGTTGGCATGCATGCGG | |
| CTCTCCACATCAATCCTTACTACGGGAAGACCTCAGCTGAAGGAATGATCTCTCATTTCGAGGCTGTCCTCCCGAT | |
| GGGTCCGACCATCATCTACAACGTGCCATCCAGGAGCGCCCAGGACATCCCCCCTGAAGTTATTCTAGCGATTTCT | |
| GGCTACACAAACATGGCGGGTGTCAAGGAATGTGTTGGGCACGAGAGGGTTAAGCACTACGCTGACAAAGGCATAA | |
| CAATTTGGAGCGGTAACGACGACGAGTGCCACGATTCTAAGTGGAAACATGGCGCTACTGGGGTCATTTCCGTTAC | |
| CAGCAACCTTGTTCCCGGGCTCATGCACAGCCTCATGTACAAAGGCGAGAACGCCACGCTGAACGAGAAGCTGTCG | |
| CCCCTGATGAAATGGCTGTTCTGCCAGCCAAATCCTATTGCCCTCAACACTGCTCTGGCTCAGCTCGGCGTGGCAA | |
| GGCCTGTCTTCAGACTGCCGTACGTTCCGCTCCCTCTTGAAAAGAGGGCCGAGTTCGTCCGGATTGTTGAGTCAAT | |
| TGGACGGGAAAATTTCGTGGGGCAGAAGGAGGCACCGGTTCTAGATGATGACGATTTCGTGTTGATCAGTAGGTAC. | |
| Amino acid-substituted DHPS gene | |
| SEQ ID 32 | |
| ATGATTTCGCCGACGAATCTCCTCCCGGCGCGGAAGATCACCCCTGTCTCAAATGGCGGCGCAGCGACGGCGAGCC | |
| CCTCTTCTCCCTCGGTGGCCGCACGGCCACGGCGACTCCCTTCAGGCCTCCAATCTGTGACTGGTAGAGGGAAGGT | |
| TTCCTTGGCAGCCATCACTCTAGATGATTACCTTCCAATGCGAAGCACTGAAGTGAAGAACCGGACATCAACAGAT | |
| GACATCACAAGGCTGAGACTAATCACAGCAGTTAAAACCCCCTATTTGCCAGATGGGAGGTTCGATCTGGAAGCAT | |
| ATGATTCTCTCATAAACATGCAGATAGAGGGTGGTGCCGAAGGCGTAATAGTTGGAGGAACAACAGGAGAGGGTCA | |
| CCTCATGAGCTGGGACGAACATATCATGCTCATTGGGCACACAGTGAACTGCTTTGGCTCTAGAATCAAAGTGATA | |
| GGCAACACAGGAAACAACTCAACCAGAGAAGCCGTCCACGCAACAGAACAGGGATTTGCTGTTGGCATGCATGCGG | |
| CTCTCCACATCAATCCTTACTACGGGAAGACCTCAGCTGAAGGAATGATCTCTCATTTCGAGGCTGTCCTCCCGAT | |
| GGGTCCGACCATCATCTACAACGTGCCATCCAGGAGCGCCCAGGACATCCCCCCTGAAGTTATTCTAGCGATTTCT | |
| GGCTACACAAACATGGCGGGTGTCAAGGAATGTGTTGGGCACGAGAGGGTTAAGCACTACGCTGACAAAGGCATAA | |
| CAATTTGGAGCGGTAACGACGACGAGTGCCACGATTCTAAGTGGAAACATGGCGCTACTGGGGTCATTTCCGTTAC | |
| CAGCAACCTTGTTCCCGGGCTCATGCACAGCCTCATGTACAAAGGCGAGAACGCCACGCTGAACGAGAAGCTGTCG | |
| CCCCTGATGAAATGGCTGTTCTGCCAGCCAAATCCTATTGCCCTCAACACTGCTCTGGCTCAGCTCGGCGTGGCAA | |
| GGCCTGTCTTCAGACTGCCGTACGTTCCGCTCCCTCTTGAAAAGAGGGCCGAGTTCGTCCGGATTGTTGAGTCAAT | |
| TGGACGGGAAAATTTCGTGGGGCAGAAGGAGGCACCGGTTCTAGATGATGACGATTTCGTGTTGATCAGTAGGTAC. | |
| Amino acid-substituted DHPS gene | |
| SEQ ID 33 | |
| ATGATTTCGCCGACGAATCTCCTCCCGGCGCGGAAGATCACCCCTGTCTCAAATGGCGGCGCAGCGACGGCGAGCC | |
| CCTCTTCTCCCTCGGTGGCCGCACGGCCACGGCGACTCCCTTCAGGCCTCCAATCTGTGACTGGTAGAGGGAAGGT | |
| TTCCTTGGCAGCCATCACTCTAGATGATTACCTTCCAATGCGAAGCACTGAAGTGAAGAACCGGACATCAACAGAT | |
| GACATCACAAGGCTGAGACTAATCACAGCAGTTAAAACCCCCTATTTGCCAGATGGGAGGTTCGATCTGGAAGCAT | |
| ATGATTCTCTCATAAACATGCAGATAGAGGGTGGTGCCGAAGGCGTAATAGTTGGAGGAACAACAGGAGAGGGTCA | |
| CCTCATGAGCTGGGACGAACATATCATGCTCATTGGGCACACAGTGAACTGCTTTGGCTCTAGAATCAAAGTGATA | |
| GGCAACACAGGAAACAACTCAACCAGAGAAGCCGTCCACGCAACAGAACAGGGATTTGCTGTTGGCATGCATGCGG | |
| CTCTCCACATCAATCCTTACTACGGGAAGACCTCAGCTGAAGGAATGATCTCTCATTTCGAGGCTGTCCTCCCGAT | |
| GGGTCCGACCATCATCTACAACGTGCCATCCAGGAGCGCCCAGGACATCCCCCCTGAAGTTATTCTAGCGATTTCT | |
| GGCTACACAAACATGGCGGGTGTCAAGGAATGTGTTGGGCACGAGAGGGTTAAGCACTACGCTGACAAAGGCATAA | |
| CAATTTGGAGCGGTAACGACGACGAGTGCCACGATTCTAAGTGGAAACATGGCGCTACTGGGGTCATTTCCGTTAC | |
| CAGCAACCTTGTTCCCGGGCTCATGCACAGCCTCATGTACAAAGGCGAGAACGCCACGCTGAACGAGAAGCTGTCG | |
| CCCCTGATGAAATGGCTGTTCTGCCAGCCAAATCCTATTGCCCTCAACACTGCTCTGGCTCAGCTCGGCGTGGCAA | |
| GGCCTGTCTTCAGACTGCCGTACGTTCCGCTCCCTCTTGAAAAGAGGGCCGAGTTCGTCCGGATTGTTGAGTCAAT | |
| TGGACGGGAAAATTTCGTGGGGCAGAAGGAGGCACCGGTTCTAGATGATGACGATTTCGTGTTGATCAGTAGGTAC. | |
| Potato DHPS gene | |
| SEQ ID 34 | |
| GATCAAGCCCAGAAGGATAGGATTACAAGTGTTGAGGGAAAAATGTCATCATCAGTTATAGGTCGCTGCCATTTCG | |
| GCGTTGAAGCTGCCGGAACAAAGAGGAGAACTGCAGTATGGAGGTCACCGAGAGCAGCTGTAATTCCAAGTTTCCA | |
| CCTCCCAATGCGTAGTTATGAAGTTAAAAATAGGACTTTTGCCGAGGATATAAAAGCACTTCGGTTGATTACCTGC | |
| AATCAAAACCCCATATCTACCAGATGGAAGATTCGATCTTGAGGCATATGACGCCTTGGTAAATTTGCAAATTGAA | |
| AATGGCGCTGAGGGTGTGATTGTTGGTGGCACTACCGGTGAAGGTCAATTGATGAAGCTGGGATGAACACATCATG | |
| CTCATTGGTCACACAGTGAACTGTTTTGGAGGATCAATCAAAGTCATTGGGAACACAGGAAGTAACTCCACGAGGG | |
| AAGCAATCCATGCGACAGAACAGGGATTTGCTGTAGGTATGCATGCAGCTCTTCACATTAATCCCTACTATGGGAA | |
| GACCTCCATAGAGGGTATGATTTCACACTTTGAAAGTGTGCTCCCTATGGGCCCTACTATCATTTATAACGTGCCA | |
| TCAAGAACTGGTCAAGACATTCCTCCACGTGTAATTCAGACATTAGCAGAGAGCCCTAATCTTGCCGGTGTCAAAG | |
| AGTGTGTTGGAAATGATAGAGTGAAGCAGTATACAAGTAATGGTTTGGTCGTGTGGAGTGGGAATGATGATGAATG | |
| CCACGACTCAAGGTGGGATCATGGTGCTGCAGGAGTTATTTCTGTTACCAGCAACTTGGTTCCTGGTCTGATGCGT | |
| GAACTAATGTTTGGAGGAAAGAACCCTGCTTTGAACTCAAAGGTAATGCCATTGGTTGAATGGTTGTTTCATGAAC | |
| CAAACCCCATTGCTCTAAATACAGCTTTAGCTCAACTCGGAGTTGTGAGGCCTGTCTTCCGCCTTCCATATGTTCC | |
| ACTTACAAGGGCAAAGAGAGAGGTGTTTGTGAAGATTGTCAAGGAAATCGGACGGGAAAACTTTATAGGGGAAAGA | |
| GATGTCCAAGTTTTGGATGATGATGATTTTGTTTTGCTTGGTAGGTATTAGCTTTTCCCCATTTGGTGTTGAGAAG | |
| TAAGTTTTTGTTGTTAGCTGCTTAATTTGAAAATTTCTTAACATGAAGAAATATATCTTCTTTTATGTTCTTTTTT | |
| GTAACCGTGCATTTTTCTTGTTTGCTCTTCTCTTCTAGGAGGCATGTACATTGTAGCTGTTAGTTTATGGTTCTAA | |
| ACTAAAGATGTGCATAATGTATCAAAAAAAAAAAAAACGGCACGAGGTAGCCTCAAAATTTCAACTTTATATTTTT | |
| GCTATTTATTGGGCCGAAACCCCCCTTCTATTATAATTATTATCTTGTCAGTTACTTAACGAACAGGTGGCAACAC | |
| TTGGGTCTAAGGGCCAAAAAGAAAATATTTCTTTTATGTTTAGACTAAGGACCATGTACCATGGATTAAATTGGCC | |
| CAAAACCAAAACCAATAGGCCCAGTTTGGCCAGGCGAACAAAAATCTGGTTGGGCCCAGCCCC. | |
| Wheat DHPS gene | |
| SEQ ID 35 | |
| CCAAACCAAAGCCCTAAATCCCTCTTTCGGAAGATAGGAGCCGCCCCCTTTCGTCGTCGCCGCCATCGCCGTTCTA | |
| CAGCTGCCGCCATGCCGTACCTCCAGCCCCCGCGCCCCCACCCCCACCCCCACCCGACCTCCCGCCTCTCGCGCGC | |
| GTCGCCGCCGTCGCCGTTCCCGTTCTTCCCCGCCGGGACATCGCGCTCCGGTCGTCTCCAACCCGTCCCAGTCTCC | |
| GGCCACTCGGCCTCGAGGGTTAGTAAAGGAAAGTTTGCAGTTGCAGCCGTCACTCTAGATGATTATCTTCCGATGC | |
| GAAGTACTGAAGTGAAAAACCGGACATCAACAGATGGAATCAAAAGCCTCAGACTAATCACAGCGGTCAAAACCCC | |
| CTATTTGCCAGATGGAAGATTTGATCTTGAAGCATACGATTCTCTGATAAACACGCAAATAAATGGCGGTGCGGAA | |
| GGTGTAATAGTTGGAGGAACAACAGGAGAAGGTCACCTCATGAGCTGGGATGAGCACATCATGCTCATCGGGCATA | |
| CTGTTAACTGCTTTGGAGCCAACATTAAAGTGATAGGCAACACGGGAAGTAACTCAACCAGAGAAGCTGTTCACGC | |
| GACAGAGCAGGGATTTGCTGTTGGCATGCATGCAGCTCTTCATGTCAATCCTTACTACGGGAAGACCTCAACTGAA | |
| GGCCTGATCTCTCATTTTAAGGAAGTCCTCCCGATGGGCCCAACAATCATTTACAACGTGCCATCCAGGACAAGTC | |
| AGGACATACCTCCACCAGTCATTGAGGCGCTTTCATGTTACTCAAACATGGCAGGAGTGAAAGAATGCGTTGGGCA | |
| TGAGCGGGTTAAGTGCTACACTGACAAAGGCATAACAATATGGAGCGGCAACGATGATGAATGCCATGACTCAAGG | |
| TGGAAGTACGGCGCTACTGGAGTCATTTCTGTGGCGAGCAACCTTGTTCCCGGTCTCATGCACAGCCTCATGTTTG | |
| AAGGGGAGAATGCGGCGCTAAATGAGAAGCTGCTGCCTCTGATGAAATGGCTGTTCTGCGAGCCTAACCCGATCGG | |
| TCTCAACACCGCCCTGGCTCAGCTCGGTGTAGTGAGGCCTGTTTTCAGGTTGCCATACACTCCCCTCCCTCTTGAG | |
| AAGAGGGTTGAGTTTGTCCGGATTGTCGAAGCCATTGGACGGGAGAACTTTGTGGGACAGAAGGAGGCCCGGGTTC | |
| TGGATGACGATGATTTCGTGTTGATCAGCAGGTATTGAATATGTGTGTGAGTTCTTTGTGTCCAGTGTATGCTGAC | |
| GAACAGCGGTAGTGTCACAGTGTTGTTTGCGGATCGTTTTAGACGCTTCGTTTGTTCACCAGTCATTTGTTGATCT | |
| TTTTCTCTGACTGCATGTTAATTTGGAACCCGTTTGGTTTATTGGCACCTTGTGATACCAGTTCCAGTAAGAAATG | |
| AGAATAGCCCGAGACATGTACAGTCGGGTTGTTACAA. | |
| Potato Mdh gene | |
| SEQ ID 36 | |
| GCACACAGTAACACACCACAACTCCTAATTTATTTTCTCCGCCGTAGCTTTTACCTTTTCCTCATTTCTTTTGTAT | |
| ATTCCGAAATGAGGACCTCCATGTTGAAATCCATCGTCCGCCGGAGCTCCACTGCCGGAGCATCCTATGTATCCCG | |
| CCGTGGATTCGCATCGGGATCCGCACCAGAGAGGAAAGTTGCAGTTTTGGGGGCAGCCGGAGGAATTGGACAGCCT | |
| TTATCTCTTCTAATGAAGCTTAACCCTTTAGTATCCAGCCTTTCACTCTACGATATCGCCGGCACTCCCGGTGTTG | |
| CCGCCGATGTTAGTCACATCAACACCAGATCTGAGGTTGTCGGTTTTGCAGGAGAAGAGCAGCTAGGGAAGGCTCT | |
| GGAAGGAGCTGATATAGTTATCATTCCTGCTGGTGTGCCCCGAAAGCCTGGTATGACCCGAGATGATCTGTTCAAC | |
| ATTAATGCGGGTATTGTTAAATCTCTTTGCACGGCCATTGCTAAGTACTGCCCCAATGCTCTGGTCAATATGATAA | |
| GCAACCCAGTGAATTCCACTGTCCCAATTGCTGCTGAGGTGTTTAAGAAGGCTGGAACTTATGATGAAAAGAAGCT | |
| CTTTGGAGTTACCATGCTTGATGTGGTTAGGGCCAAGACATTTTATGCTGGAAAAGCTAAAGTAAATGTTGCTGAG | |
| GTCAATCTTCCGGTAGTTGGTGGTCATGCTGGCATAACTATCCTCCCATTATTTTCTCAAGCCACTCCAAAGGCAA | |
| ATCTGTCAAATGAGGAAATTGTTGCCCTCACAAAGCGAACACAAGATGGTGGCACAGAAGTCGTGGAGGCCAAGGC | |
| TGGAAAGGGTTCAGCCACCCTCTCAATGGCCTATGCTGGGGCTATTTTTGCCGATGCTTGCTTGAAGGGTTTGAAT | |
| GGAGTTCCCGATGTTGTTGAATGTGCTTTTGTGCAGTCCAATGTTACCGAGCTTCCCTTCTTTGCATCCAAGGTGA | |
| GACTTGGGAAAAATGGAGTGGAGGAAGTCCTTGGGTTGGGTCCACTTAACGAGTATGAGAAGCAAGGACTTGAGGC | |
| TCTGAAGCCAGAGCTGCTCTCCTCCATTGAAAAGGGAATCAAGTTTGCCAAAGAGAACTAAAAACAAAATTATGGT | |
| GTAGTTTTCTAGAGTGACAGTTTTGGATCCTTTTGTGCAATTGTGTTGTATCCTTTGCAAGTTTCTTGCAGCCGGA | |
| GGCTTAGATTTAGCTCTTTTGATATATACCCAACATTTCTACAAAATAATGTATGGCAAACTGGCGTCTATGTCAT | |
| TTGCCTTAGTGTGGAGGTGTTATTCTCACATGGATCGTTTTCCAATTATGGTTAGAAAGCAGACAATTGATTTGAA | |
| ATGAAGAATGTTAATGACCAATTACTGT. | |
| Sense fragment of Mdh gene | |
| SEQ ID 37 | |
| TTCACTCTACGATATCGCCGGCACTCCCGGTGTTGCCGCCGATGTTAGTCACATCAACACCAGATCTGAGGTTGTC | |
| GGTTTTGCAGGAGAAGAGCAGCTAGGGAAGGCTCTGGAAGGAGCTGATATAGTTATCATTCCTGCTGGTGTGCCCC | |
| GAAAGCCTGGTATGACCCGAGATGATCTGTTCAACATTAATGCGGGTATTGTTAAATCTCTTTGCACGGCCATTGC | |
| TAAGTACTGCCCCAATGCTCTGGTCAATATGATAAGCAACCCAGTGAATTCCACTGTCCCAATTGCTGCTGAGGTG | |
| TTTAAGAAGGCTGGAACTTATGATGAAAAGAAGCTCTTTGGAGTTACCATGCTTGATGTGGTTAGGGCCAAGACAT | |
| TTTATGCTGGAAAAGCTAAAGTAAATGTTGCTGAGGTCAATCTTCCGGTAGTTGGTGGTCATGCTGGCATAACTAT | |
| CCTCCCATTATTTTCTCAAGCCACTCCAAAGGCAAATCTGTCAAATGAGGAAATTGTTGCCCTCACAAAGCGAACA | |
| CAAGATGGTGGCACAGAAGTCGTGGAGGCCAAGGCTGGAAAGGGTTCAGCCACCCTCTCAATGGCCTATGCTGGGG | |
| CTATTTTTGCCGATGCTTGCTTGAAGGGTTTGAATGGAGTTCCCGATGTTGTTGAATGTGCTTTTGTGCAGTCCAA | |
| TGTTACCGAGCTTCCCTTCTTTGCATCCAAGGTGAGACTTGGGAAAAATGGAGTGGAGGAAGTCCTTGGGTTGGGT | |
| CCACTTAACGAGTATGAGAAGCAAGGACTTGAGGCTCTGAAGCCAGAGCTGCTCTCCTCCATTGAAAAGGGAATCA | |
| AGTTTGCCAAAGAGAACTAAAAACAAAATTATGGTGTAGTTTTCTAGAGTGACAGTTTT. | |
| Antisense fragment of Mdh gene | |
| SEQ ID 38 | |
| AAAACTGTCACTCTAGAAAACTACACCATAATTTTGTTTTTAGTTCTCTTTGGCAAACTTGATTCCCTTTTCAATG | |
| GAGGAGAGCAGCTCTGGCTTCAGAGCCTCAAGTCCTTGCTTCTCATACTCGTTAAGTGGACCCAACCCAAGGACTT | |
| CCTCCACTCCATTTTTCCCAAGTCTCACCTTGGATGCAAAGAAGGGAAGCTCGGTAACATTGGACTGCACAAAAGC | |
| ACATTCAACAACATCGGGAACTCCATTCAAACCCTTCAAGCAAGCATCGGCAAAAATAGCCCCAGCATAGGCCATT | |
| GAGAGGGTGGCTGAACCCTTTCCAGCCTTGGCCTCCACGACTTCTGTGCCACCATCTTGTGTTCGCTTTGTGAGGG | |
| CAACAATTTCCTCATTTGACAGATTTGCCTTTGGAGTGGCTTGAGAAAATAATGGGAGGATAGTTATGCCAGCATG | |
| ACCACCAACTACCGGAAGATTGACCTCAGCAACATTTACTTTAGCTTTTCCAGCATAAAATGTCTTGGCCCTAACC | |
| ACATCAAGCATGGTAACTCCAAAGAGCTTCTTTTCATCATAAGTTCCAGCCTTCTTAAACACCTCAGCAGCAATTG | |
| GGACAGTGGAATTCACTGGGTTGCTTATCATATTGACCAGAGCATTGGGGCAGTACTTAGCAATGGCCGTGCAAAG | |
| AGATTTAACAATACCCGCATTAATGTTGAACAGATCATCTCGGGTCATACCAGGCTTTCGGGGCACACCAGCAGGA | |
| ATGATAACTATATCAGCTCCTTCCAGAGCCTTCCCTAGCTGCTCTTCTCCTGCAAAACCGACAACCTCAGATCTGG | |
| TGTTGATGTGACTAACATCGGCGGCAACACCGGGAGTGCCGGCGATATCGTAGAGTGAAAGGCTGGATACTAAAGG | |
| GTTAAGCTTCATTAGAAGAGATAAAGGCTGTCCAATTCCTCCGGCTGCCCCCAAAACTGCAACTTTCCTCTCTGGT | |
| GCGGATCACGATGCGAATCCACGGCGGGATACATAGGATGCTCCGGCAGTGGAGCTCCGGCGGACGATGGATTTCA | |
| ACATGGAGGTCCTCATTTCGGAATATACAAAAGAAATGAGGAAAAGGTAAAAGCTACGGCGGAGAAAATAAATTAG | |
| GAGTTGTGGTGTGTTACTGTGTGC. | |

What is claimed is:

1. A method for reducing acrylamide levels in the heat-processed product of a plant, comprising reducing the levels of asparagine in cells of the plant.

2. The method of claim 1, wherein the levels of asparagine are reduced by modifying the expression of at least one target gene that is involved in the accumulation of asparagine.

3. The method of claim 2, comprising upregulating the expression of a target gene selected from the group consisting of (i) a glutamine-rich storage protein gene, and (ii) a lysine-feedback-less-sensitive dihydrodipicolinate synthase gene.

4. The method of claim 3, wherein the glutamine-rich storage protein target gene encodes a wheat glutenin.

5. The method of claim 2, comprising downregulating the expression of a target gene selected from the group consisting of (i) a glutamine synthetase gene, (ii) a mitochondrial malate dehydrogenase gene, (iii) an aspartate aminotransferase, and (iv) a lysine feed-back less-sensitive form of an aspartate kinase gene.

6. The method of claim 5, wherein the step of downregulating the expression of a gene comprises introducing into cells of the plant an expression cassette which comprises one or more copies of a target gene sequence fragment operably linked to at least one promoter, wherein expression of the target gene fragment(s) produces at least one of a single-stranded RNA transcript or a double-stranded RNA transcript.

7. The method of claim 6, wherein the target gene sequence fragment is (i) operably linked to a first promoter at the 5′-end of the fragment, (ii) operably linked to a second promoter at the 3′-end of the fragment, but wherein the fragment (ii) is not operably linked to a terminator.

8. The method of claim 6, wherein the target gene sequence fragment comprises at least about 20-50 contiguous nucleotides of a sequence of the target gene.

9. The method of claim 6, wherein the expression cassette comprises at least two copies of the target gene sequence fragment.

10. The method of claim 9, wherein the copies of the target gene sequence fragment are oriented in the expression cassette as (i) inverted repeats, or (ii) direct repeats.

11. The method of claim 10, wherein the inverted repeat sequence, or the direct repeat sequence, is (i) operably linked to a first promoter at the 5′-end of the repeat sequence, (ii) operably linked to a second promoter at the 3′-end of the repeat sequence, but wherein the repeat sequence (ii) is not operably linked to a terminator.

12. The method of claim 6, wherein the target gene encodes aspartate aminotransferase, and wherein the target gene sequence fragment shares at least 70% identity with a sequence depicted in any one of SEQ ID NOs: 1, 2, 3, 4, or 5.

13. The method of claim 6, wherein the target gene encodes a lysine feed-back less-sensitive form aspartate kinase, and wherein the target gene sequence fragment shares at least 70% identity with a sequence depicted in any one of SEQ ID NOs: 14, 15, 16, 17, or 18.

14. The method of claim 6, wherein the promoter is selected from the group consisting of (i) a potato granule bound starch synthase gene, (ii) a potato ADP glucose pyrophosphorylase gene, (iii) a potato ubiquitin-7 gene, (iv) a potato patatin gene, (v) a potato flavonoid mono-oxygenase gene.

15. The method of claim 14, wherein (i) the promoter of the potato granule bound starch synthase gene shares at least 70% identity with at least part of SEQ ID NO: 12, or (ii) the promoter of a potato ADP glucose pyrophosphorylase gene shares at least 70% identity with at least part of SEQ ID NO: 11, or (iii) the promoter of a potato flavonoid mono-oxygenase gene shares at least 70% identity with at least part of SEQ ID NO: 19.

16. The method of claim 1, wherein the plant is selected from the group consisting of a potato plant, a wild potato plant, a sweet potato plant, a yam plant, a corn plant, a coffee plant or tree, a cocoa plant or tree, wheat, maize, oats, sorghum, or barley.

17. The method of claim 1, wherein the plant is a crop, and wherein the cells of the plant are from a tuber, root, bean, or seed.

18. The method of claim 1, further comprising modifying the expression of at least one gene involved in the accumulation of reducing sugars.

19. The method of claim 18, wherein the gene involved in the accumulation of reducing sugars is downregulated in expression and selected from the group consisting of (i) a starch-associated R1 gene, and (ii) a starch-associated phosphorylase-L gene.

20. The method of claim 19, wherein downregulation comprises introducing into cells of the plant an expression cassette which comprises one or more copies of a fragment of a starch degradation gene operably linked to at least one promoter, wherein expression of the fragment(s) produces at least one of a single-stranded RNA transcript or a double-stranded RNA transcript.

21. The method of claim 18, wherein the gene involved in the accumulation of reducing sugars is upregulated in expression and selected from the group consisting of (i) an SnRk1 gene, (ii) a Dofl gene, and (iii) an Mdh gene.

22. The method of claim 1, further comprising obtaining a plant product from the plant with reduced asparagine levels, wherein the product has a lower concentration of acrylamide than an equivalent product from a plant of the same species that has not had its asparagine levels reduced.

23. The method of claim 22, wherein the heat-processed product is from a potato tuber.

24. The method of claim 23, wherein the heat-processed product is a French fry or chip processed from the potato.

25. The method of claim 23, wherein the potato, when stored between 4° C. and 12° C. for about one to thirty weeks, contains a glucose level that is less than 50% of the glucose level of an unmodified tuber of the same species stored under the same storage conditions as the potato.

26. An asparagine-reduced potato product that has a level of acrylamide that is at least 10% lower than the level of acrylamide from a non-asparagine-reduced potato product.

27. The asparagine-reduced potato product of claim 26, wherein the product has a level of acrylamide (in parts per billion) selected from the group consisting of (i) 1-20 ppb, (ii) 20-40 ppb, (iii) 40-60 ppb, (iv) 60-80 ppb, and (v) 80-100 ppb of acrylamide.

28. The asparagine-reduced potato product of claim 27, wherein (A) the product is selected from the group consisting of a fried fry, an oven-baked fry, potato chip, hash brown, and tater totters, and (B) the product has a level of acrylamide (in parts per billion) selected from the group consisting of 1-20 ppb, 20-40 ppb, 40-60 ppb, 60-80 ppb, 80-100 ppb, 100-120 ppb, 120-140 ppb, 140-160 ppb, 160-180 ppb, and 180-200 ppb of acrylamide.

29. The method of claim 16, wherein ground coffee obtained from the coffee plant has a level of acrylamide that is at least 10% lower than the level of acrylamide of ground coffee from an untransformed plant counterpart, and wherein the acrylamide level is below 100 ppb.

30. Roasted coffee from beans with reduced asparagine levels with levels of acrylamide below 100 ppb.

31. The method of claim 16, wherein the plant product is wheat bread that has a level of acrylamide that is at least 10% lower than the level of acrylamide of wheat bread from an untransformed plant counterpart, and wherein the level of acrylamide is below 50 ppb.

32. Wheat bread from flour with reduced asparagine levels with levels of acrylamide below 50 ppb.

33. Heat-processed products from potato tubers with reduced asparagine levels with levels of acrylamide below 100 ppb.


SEQUENCE LISTING (38 sequences)

SEQ ID NO: 1 - DNA, 1763 bp, Solanum sp.
aaaagccgttgataaaccattcgttcgcactgacggctcgctcagtccactactcttacgtctcctatatataaagtccatctctatcaaccacttcgacaaacacgccatcttcctctccgatttctctaaatactctctccgctccggccatggcgatccgtgccgcgatttccggtagtcatctcaagcttagctcgtcgtcgctcggagcgcgatccttgtcgtcgtggtggcggaacgttgagccggcacctaaagatcctatcctcggcgttactgaagctttcctcgccgatcctagccctgataaagtcaatgttggcgttggagcttacagggatgacaatggaaaacccgtggtactggagtgtgtcagagaagcagagcgaaggatcgctggcggtttcaacatggaatatcttcctatgggaggtagtgtcaacatgatccaggagtcgctgaagttagcctatggggagaactcagacttgataaaagataaacgcattgctgcaattcaagcattatctggaactggcgcatgccgaatttttgcggacttccaaaagcgcttttgtcctgattcacagatttatattccagttcctacatggtctaatcatcataacatttggagagatgctcatgtccctgagagaacatatcactattatcatgctgaatcaaagggcttggactttgctgcaatgatggatgatataaagaatgccccaaaaggatcattctttctgcttcatgcttgtgctcataatcctactggggtggatccgacagaggaacaatggagggagatctcgcaccagttcaaggtgaagggacattttgctttgtttgatatggcctatcaaggatttgcaagtgggtatccagagaaggatgctaaggcaatcaggatatttcttgaggatggtcatccgatagcgtgtgcccaatcatatgcaaaaaatatggggctatatggccagagagttggttgcctaagtgttgtctgtgaggatgaaaaacaagcagtggcagtgaaaagtcagttgcagcaacttgccaggcccatgtacagtaatccacctgttcatggtgctcttgttgtttctaccatccttggagatccaaacttgaaaaatctatggctcggggaagtgaagggcatggctgatcgtatcatcgggatgagaactgctttaagggaaaatcttgagaagttgggctcacctctatcttgggaacacataaccaaccagattggcatgttctgctacagtgggatgacaccggaacaagtagaccgattgacaaaagagtatcacatctacatgactcgtaatggtcgtatcagtatggcaggagttactactggaaatgttggttacttggcaaacgctattcatgaggttactaaatctgcttaagctttaacatcatggattcaataatcagttacggaaaggctgtggaaattcattttttgtgatcggaaagatcacaacatgttgtaatagttgagttaaaacaatcgtactgaggggaggggggaataaatagaaccattttaagggatagaagtcattagcagccgtcgaggtggagttgtagttattttagggtacattacagacattaatcatctctagtgtcaaggaactgctgtttttcatatacaacagcgtagttacatgatatatatgccaacattttgaagtattgttactgttttatggcgtcatggatggaagtaattattg

SEQ ID NO: 2 - DNA, 1424 bp, Solanum sp.
ctccataacatcgttgaacttgttctctgaaattccaactccaatggcagattctgtgtttgctcatgttgttcgtgctcctgaagatcccatcttaggggtaacagttgcttataacaaagatagcagcccagtcaaagtgaatttgggtgttggcgcatatcgaactgaggaaggaaagccccttgttcttaatgtcgtgagacgagctgaacaaatgcttgtcaatgacacgtctcgcgtgaaggaatatctctcaattactggacttgcggattttaacaaattgagtgccaagcttatatttggtgctgacagccctgccattaaagagaatagggtgaccactgttcagtgcttgtcgggcactggttctttgagggttggtgctgagtttctggccaggcattatcatgaacatactatatatataccacagccaacatggggaaaccatcctaagattttcactttagctgggctttcagtaaaatattatcgttactatgacccagcaacacgaggcctggattttcaaggattattggatgatcttacggctgcaccctctggtgcaatagttcttctccatgcttgtgctcataacccaactggcgttgatccaacaattgaccagtgggaaaaaatcaggaagttgatgaggtccaaggggttgttacctttctttgacagtgcttatcagggttttgccagtgggaacctagatgcagatgcacaatctgttcgcatgtttgtggctgatggtggtgaatgtcttgcagctcagagttatgccaaaaacatgggactgtatggagagcgtgtcggtgcccttagcattgtttgcaaagatagcaatgttgcaagcaaagtcgaaagccagctaaagatggttataaggccaatgtactcaaatccaccgattcatggtgcgtctattgttgccactatactcaaggacagacaaatgtatgacgaatggacagttgagctgaaagcaatggccgaccgaattattagcatgcgccagcaactctatgatgccatgcaagctcgaggtactccaggtgattggagtcatatcatcaagcaaattgggatgtttactttcacaggattgaatgttgagcaagtctcgttcatgactagagagtatcacatttacatgacatctgatgggagaattagcatggcaggtcttagttctcggacagttcctcatcttgctgatgccatgcatgctgctgttactagggcggcctagattagttgcattcaagagcagttttattaatattgaatctactttccatgatcgcctccttatcctttgatgttaaataagacgaatgtgtctatgtagacataatttatatttggaaatttcattctaaaggaatttgatgaggtcttctgctaagggg

SEQ ID NO: 3 - DNA, 1200 bp, Solanum sp.
gataaagataggcgtactatagcaagcaaaagttccatactactaaaatgcacacacaacaatcaccgtcaccctccgccgatcggaggttgagtgttctggcgaggcaccttgaaccgtcgtcctccaccgccgttgaaggtcacagcaaccactccattgtcggtgctcctacatcaggatatgatggaaaacagtctgtattctctcacatcgttcgcgctcccgaagatcctattctcggcgttactgttgcatacaacaaagataccagccccgtgaagttgaacttgggagttggtgcatataggacagaggaaggaaaacctcttgttttgaatgttgttagaaaagcagaacagctactagtaaatgataggtctcgcataaaagagtaccttccaattgttgggctggcagacttcaataaattgagtgctaagctgatacttggtgctgacagccctgctattcaagagaacagagtaacaactgtgcagtgcttgtctggcacaggctcattgagggttggaggtgaatttttggctcaacattatcatcaacgtactatttatattccccaaccgacatggggaaaccacccaaaaattttcactttagctggattgtcagttaagagttaccgctactatgatccagcaacccgtggactccattttcaaggtttgttggaagaccttggatctgctccgtcaggagcgatagtgttacttcacgcttgtgcacataaccccactggtgtggatccaaccaaagatcagtgggagcaaattaggaaattgatgagatcaaaaggattgctacccttctttgatagtgcatatcaggggtttgctagtggaagcctagatatagatgcacagtctgttcgcatgtttgtggcagatggaggtgaagtacttgttgctcaaagttatgcaaagaatatggggctttatggtgaacgtgtcggagctttaagcatagtgtgcagaaatgctgatgtagctagcagagttgagagccagtttgaagttggggtgtatttagggccaaatggtattccccaagttcccaccaaatttccacgggtggcgtcaactagtttgcttacaaatcctccaaagacaggaaaacttgttaccactgaatggcactcttggaatttgaaaagccgatgggttgatcgaattcttccag

SEQ ID NO: 4 - DNA, 1513 bp, Solanum sp.
gctaattttacaatacaagcaaaaaaacagaacagaagagagcaagaaagatgaacatgtggagtagacatgttagatggctgtctacctcagtagcaacaaataggaaaaacaatatgggatggtgggaccatgtgcagccagctcctaaagacccaatcaccagtgtcactcaggcttttctttctgacccaactcctctcaagctcaatcttgggagcttacagagatgataaaggaaaaccagtaactcttgagtgtgttagaagagctgctgaaaagatttctggctgtgaaatcttggaatcaactaaagcaacaacaaaatcaaagtttgtgcaggactgtgttaaattggcttatggaaatgactctagtgttgttagagaagacaggtttgctggtgttccagctctttctgggactggtgcgtgccggctgtttgcggagttccaaaggcgcttctatcctgactcacaaatgtttctaccaatgccaacttggtccaaccaccacgacatttggagggattctcaagtttgtacaagaacctaccactattatgatcctggtacaaaaggattgcgatttcaagccctcgtaaatgatatcaagaatgctccagatcgttcctttttcttacttcatccttgtgctcataccccaactggtgtcgaccccaactatgagcagtggaaagaaatctcacacatatttaagatcaagaatcattttcctttttttgatatggcttatcaaggaattgctagcggagatgttgagagagatgccactgctattcgaatatttctagaagatggacatctattgggctgtgcccaatctttttccaaaaacatgggcttgtatgaacaccgagtaggttgtgtcagtattgtctcctgggatgataagcaggccacagcaatcaagagccagttgcagcgaattgtcagggcaatgtacagtagctctccggttcatggcccattattggtgtcaacaatcttgaatgatgctgatttaaaggcactttgggaagaggaagttaaggtcatggtggatcgcttaatcagtatgagaatcacattacgtcaaactcttgaggagttgaactcatcttcgagctgggaacatataacgaagcaggttggaatgttttacttctctggtttatcccctgaggaagttaaccacttgcagagggattttcatatatacatgactaacgatgggcgcatcagcatggcaggggtaacaagaagcaatgtagactacttggcaagtgcaatccatgaagttaccagtgtaaataatagcaactgtattgattcaaagcattttgaatttgtagtttagcaattcttattgagatcttgtgcagataaccattgtgtaatactgttagaggttgttcggtaggatgtattagagaaaataatacatgtatacttattcagtactattcttatacataacaatcaatgatattagcaatataagggttattaat

SEQ ID NO: 5 - DNA, 1072 bp, Solanum sp.
ttaccatcgatcagttcttccctctgtggtagctttcctgtaaacccaaagtcccgaaacccttgtatgccctcttcttcttttgcgatggcttccacaatgttctctctagcttctgtcacaccttcagcttcaatttccttgcaagataatctcaagccaaagctgaagctagggacttctagccaaagggcctttcccaggaaggacttcatgaaggcaaagtcaaatggtcggactactatggctgttgctgttaacgtctctcgatttgagggaataacaatggctccccctgaccccattcttggagtctctgaagcattcaaggctgatacaaatgaactgaagcttaaccttggagttggagcttaccgcacagaggatcttcaaccatatgtcctcaatgttgttaagaaagcagagaaccttatcctggagagaggagaaaacaaagagtatcttccaatagaaggtttggctgcattcaacaaagtcacagcagagttattgtttggaaaagataacctggtgcttcagcaacaaagggtggctacgattcaaggtctatcaggaactgggtcattgcgtattgctgcagcactgatagagcgttacttccctggctctaagattttgatatcatctccaacatggggaaatcataaaaacatttttaatgatgccagagtaccttggtctgaatatcgatattatgatcccaaaactgttggccttgactttgttgggatgatagaagatataaacggctgccccagaaaggatcacctttatccttgctccacggctgtgcacacaacccaactggtattgattcccacaccttgagcaatgggaaaagatcgctgatggtattcaggagaagaaccacattccattttttgatgttgcctaccaaggattttgcagtggcagccttgacgaagatgcctcatctgtgagaatgtttgctgcacgtggtatggagcttttgggtgctcaatcatatagtaaaaatctgggtctctatggagaaggatgggggctataaatgtctttgctcatctgctga

SEQ ID NO: 6 - DNA, 181 bp, Artificial Sequence
  Description of Artificial Sequence Synthetic polynucleotide
tgcttcatgcttgtgctcataatcctactggggtggatccgacagaggaacaatggagggagatctcgcaccagttcaaggtgaagggacattttgctttgtttgatatggcctatcaaggatttgcaagtgggtatccagagaaggatgctaaggcaatcaggatatttcttgaggatgg

SEQ ID NO: 7 - DNA, 181 bp, Artificial Sequence
  Description of Artificial Sequence Synthetic polynucleotide
ttctccatgcttgtgctcataacccaactggcgttgatccaacaattgaccagtgggaaaaaatcaggaagttgatgaggtccaaggggttgttacctttctttgacagtgcttatcagggttttgccagtgggaacctagatgcagatgcacaatctgttcgcatgtttgtggctgatgg

SEQ ID NO: 8 - DNA, 181 bp, Artificial Sequence
  Description of Artificial Sequence Synthetic polynucleotide
tacttcacgcttgtgcacataaccccactggtgtggatccaaccaaagatcagtgggagcaaattaggaaattgatgagatcaaaaggattgctacccttctttgatagtgcatatcaggggtttgctagtggaagcctagatatagatgcacagtctgttcgcatgtttgtggcagatgg

SEQ ID NO: 9 - DNA, 181 bp, Artificial Sequence
  Description of Artificial Sequence Synthetic polynucleotide
tacttcatccttgtgctcataccccaactggtgtcgaccccaactatgagcagtggaaagaaatctcacacatatttaagatcaagaatcattttcctttttttgatatggcttatcaaggaattgctagcggagatgttgagagagatgccactgctattcgaatatttctagaagatgg

SEQ ID NO: 10 - DNA, 183 bp, Artificial Sequence
  Description of Artificial Sequence Synthetic polynucleotide
tgctccacggctgtgcacacaacccaactggtattgattcccacaccttgagcaatgggaaaagatcgctgatggtattcaggagaagaaccacattccattttttgatgttgcctaccaaggattttgcagtggcagccttgacgaagatgcctcatctgtgagaatgtttgctgcacgtgg

SEQ ID NO: 11 - DNA, 2262 bp, Solanum sp.
caagtgtctgagacaaccaaaactgaaagtgggaaaccaaactctaagtcaaagactttatatacaaaatggtataaatataattatttaatttactatcgggttatcgattaacccgttaagaaaaaacttcaaaccgttaagaaccgataacccgataacaaaaaaaatctaaatcgttatcaaaaccgctaaactaataacccaatattgataaaccaataactttttttattcgggttatcggtttcagttctgtttggaacaatcctagtgtcctaattattgttttgagaaccaagaaaacaaaaacttacgtcgcaaatatttcagtaaatacttgtatatctcagtgataattgatttccaacatgtataattatcatttacgtaataatagatggtttccgaaacttacgcttcccttttttcttttgcagtcgtatggaataaaagttggatatggaggcattcccgggccttcaggtggaagagacggagctgcttcacaaggagggggttgttgtacttgaaaatgggcatttattgttcgcaaacctatcatgttcctatggttgtttatttgtagtttggtgttcttaatatcgagtgttctttagtttgttccttttaatgaaaggataatatctgtgcaaaaataagtaaattcggtacataaagacatttttttttgcattttctgtttatggagttgtcaaatgtgaatttatttcatagcatgtgagtttcctctcctttttcatgtgcccttgggccttgcatgtttcttgcaccgcagtgtgccagggctgtcggcagatggacataaatggcacaccgctcggctcgtggaaagagtatggtcagtttcattgataagtatttactcgtattcggtgtttacatcaagttaatatgttcaaacacatgtgatatcatacatccattagttaagtataaatgccaactttttacttgaatcgccgaataaatttacttacgtccaatatttagttttgtgtgtcaaacatatcatgcactatttgattaagaataaataaacgatgtgtaatttgaaaaccaattagaaaagaagtatgacgggattgatgttctgtgaaatcactggtaaattggacggacgatgaaatttgatcgtccatttaagcatagcaacatgggtctttagtcatcatcattatgttataattattttcttgaaacttgatacaccaactttcattgggaaagtgacagcatagtataaactataatatcaattctggcaatttcgaattattccaaatctcttttgtcatttcatttcctcccctatgtctgcaagtaccaattatttaagtacaaaaaatcttgattaaacaatttattttctcactaataatcacatttaatcatcaacggttcatacacgtctgtcactctttttttattctctcaagcgcatgtgatcataccaattatttaaatacaaaaaatcttgattaaacaattcagtttctcactaataatcacatttaatcatcaacggttcatacacatccgtcactctttttttattctctcaagcgcatgtgatcataccaattatttaaatacaaaaaatcttgattaaacaattcattttctcactaataatcacatttaatcatcaacggtttatacacgtccgccactctttttttattctctcaagcgtatgtgatcatatctaactctcgtgcaaacaagtgaaatgacgttcactaataaataatcttttgaatactttgttcagtttaatttatttaatttgataagaatttttttattattgaatttttattgttttaaattaaaaataagttaaatatatcaaaatatcttttaattttatttttgaaaaataacgtagttcaaacaaattaaaattgagtaactgtttttcgaaaaataatgattctaatagtatattctttttcatcattagatattttttttaagctaagtacaaaagtcatatttcaatccccaaaatagcctcaatcacaagaaatgcttaaatccccaaaataccctcaatcacaagacgtgtgtaccaatcatacctatggtcctctcgtaaattccgacaaaatcaggtctataaagttacccttgatatcagtattataaaactaaaaatctcagctgtaattcaagtgcaatcacactctaccacacactctctagtagagagatcagttgataacaagcttgttaacg

SEQ ID NO: 12 - DNA, 688 bp, Solanum sp.
cgaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcacg

SEQ ID NO: 13 - DNA, 360 bp, Solanum sp.
ttgattttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatagg

SEQ ID NO: 14 - DNA, 1362 bp, Artificial Sequence
  Description of Artificial Sequence Synthetic polynucleotide
ggatccatgtctgaaattgttgtctccaaatttggcggtactagcgtagccgattttgacgccatgaaccgcagcgctgatattgtgctttctgatgccaacgtgcgtttagttgtcctctcggcttctgctggtatcactaatctgctggtcgctttagctgaaggactggaaccttgcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtgcgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgatgtcccgggcatctacaccaccgatccacgtgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacacaatctctgctgatggagctttccgcactgtgtcgggtggaggtggaagaaggtctggcgctggtcgcgttgattggcaatgacctgtcaaaagcgtgcgccgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtgagaattc

SEQ ID NO: 15 - DNA, 1604 bp, Artificial Sequence
  Description of Artificial Sequence Synthetic polynucleotide
ggatcctatttttacaacaattaccaacaacaacaaacaacaaacaacattacaattactatttacaattacaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcatgtctgaaattgttgtctccaaatttggcggtactagcgtagccgattttgacgccatgaaccgcagcgctgatattgtgctttctgatgccaacgtgcgtttagttgtcctctcggcttctgctggtatcactaatctgctggtcgctttagctgaaggactggaaccttgcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtgcgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgatgtcccgggcatctacaccaccgatccacgtgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacacaatctctgctgatggagctttccgcactgtgtcgggtggaggtggaagaaggtctggcgctggtcgcgttgattggcaatgacctgtcaaaagcgtgcgccgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtgagaattc

SEQ ID NO: 16 - DNA, 1604 bp, Artificial Sequence
  Description of Artificial Sequence Synthetic polynucleotide
ggatcctatttttacaacaattaccaacaacaacaaacaacaaacaacattacaattactatttacaattacaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcatgtcagaaatagtcgtaagcaaatttggtggtacttctgtagctgattttgatgctatgaacagatctgctgatatagttttatcagacgctaatgtacgtctcgttgttttgagtgcatccgccggtatcacaaacttgttagtcgcattggcagaaggtcttgaaccatgtgaaagatttgagaaacttgacgccattagaaatatccaattcgccatactagaaagacttagatacccaaacgtaattcgtgaagaaatagagcgtcttttagaaaatataacagttcttgccgaagctgctgcccttgctacatccccagctcttactgatgaattagtgtcacacggagaattaatgtccaccctactgtttgttgaaattcttagagaaagagatgtccaagctcaatggtttgatgtaagaaaagttatgaggacaaatgatagattcggacgtgcagaacctgatattgccgctctcgctgaacttgccgcattacaattactcccccgactaaatgaagggcttgttattacacaaggttttatagggtcagaaaacaaaggtcgaacaacaactcttggtagaggaggttccgattacactgctgcactacttgccgaagctctacatgcctcaagagttgatatatggacagacgtgccaggaatttatactactgatcctagggttgttagcgccgcaaaaagaatcgacgaaattgcattcgcagaggctgcagaaatggctacatttggagctaaagtgttacatccagctactctactacccgcagttaggtccgacattccagtgttcgttggaagttcaaaagatccaagagcaggcgggacacttgtatgtaataaaaccgaaaatccacctttattcagagcacttgcccttagacgtaaccaaacactgttgactctccattctttaaacatgctacattcccgaggtttccttgcagaagttttcggtatccttgctagacataatatttcagtagatcttataaccacaagcgaagtttcagttgccctcatattagacactacaggttccacatcaaccggagatactttactaacacaatcacttttaatggaactatcagcactctgtcgagttgaagtcgaagaaggactagcattagtagccctcatcggaaatgaccttagcaaagcttgtgcagtggggaaggaagtttttggagtcttggagccttttaatataagaatgatatgctacggcgcctcttcacacaatctttgttttttagtgcctggagaagatgcagaacaagttgtacagaaacttcattctaatctctttgaatgagaattc

SEQ ID NO: 17 - DNA, 1272 bp, Streptomyces albulus
atgggccttgtcgtgcagaagtacggcggctcatccgttgcggatgccgagggcatcaagcgcgttgccaagcgaatcgtcgaggccaagaagaacggcaaccaggttgtcgtcgtggtctcggcgatgggcgacacgacggacgagctgatcgatctcgcgcaggaagtgtccccgatcccgtcgggacgcgagttcgacatgctgctgaccgccggagagcggatctccatggccctgctggcgatggcgatcaaaaacctcggccatgaggcgcagtccttcaccggtagccaggccggtgtgatcactgactccgtgcacaacaaggcacggatcatcgatgtcacgccgggccgcatcaaggcgtccctcgacgagggcaacatcgccatcgtcgccggattccagggcgtgtcccaggacaagaaggacatcacgacgctcgggcgcggtggttcggacaccaccgcggtggcgctcgccgccgccctgaacgccgatgtctgcgagatctacaccgacgtcgacggcgtcttcaccgccgacccgcgggtcgtgaagaaggcccggaagatcgagtggatctccttcgaggacatgctggagctggccagctccggctccaaggtgctgctgcaccgctgcgtcgagtacgcacgccgttacaacatcccgatccacgtccgctcctccttctcggggctgcagggcacatgggtcagcaacgaaccgcaaggggacaggccgatggaacaggcgatcatctcgggcgtcgcacacgacacctccgaggcgaaggtcacggtcgtcggggtcccggacaagccgggcgaggccgcgcggatcttccgtgccatcgccgactccgaggtcaacatcgacatggtcgtccagaacgtgtcggcggcctccaccggtctgacggacatctccttcacgctgccgaaggccgagggccgcaaggccgtcgccgcgctggagaagacccgggccgcggtcggcttcgactcgctccgctacgacgaccagatcgccaagatctcgctggtcggcgcgggcatgaagaccaaccccggcgtcaccgcgacgttcttcgaggcgctgtcgaacgcgggcgtgaacatcgagctcatctcgacctccgagatccgcatctcggtcgtcacccgtgccgatgacgtcaacgaggccgtccaggcggtgcacagcgccttcggcctcgacagtgagaccgacgaagcagtcgtctacggcggcaccgggcgatga

SEQ ID NO: 18 - DNA, 1269 bp, Artificial Sequence
  Description of Artificial Sequence Synthetic polynucleotide
atgggacttgttgtacaaaaatatggaggaagttcagttgccgacgccgagggaataaaacgtgtagcaaagcgaattgtcgaagcaaaaaaaaacggaaatcaagtagttgtagttgtatcagcaatgggagatactaccgatgaacttattgatctagcacaggaagtttcaccaattcctagcggtagagaatttgatatgcttttgactgcaggcgaacgtataagcatggcattactagcaatggctattaaaaatctaggacatgaagctcagtcatttacaggctctcaagcaggcgttataaccgattctgttcataataaagctcgaattatagatgttacaccaggtcgaatcaaagcaagtttagatgaaggtaatatagctatagtggccggatttcaaggagtttcacaagacaagaaggacattactactctaggtaggggcggatctgatactactgctgttgcacttgctgctgccttaaatgctgatgtttgcgaaatatacacagatgtagatggcgtttttactgctgacccaagagttgttaaaaaagcaagaaaaatagaatggatctcatttgaagatatgttggagctcgcttcctcaggtagtaaagtactgttacatcgttgcgtcgaatatgctcgaagatataatataccaatccacgttcgatccagcttttcaggacttcaaggtacatgggtttcaaacgagccacaaggtgatagaccaatggaacaagcaattattagtggtgttgcacatgatacttccgaagctaaagttaccgtagttggcgtaccagataaaccaggagaagctgcaagaatttttagagctatagcagattcagaagttaatatagatatggttgttcagaatgttagtgctgcttcaacaggattgactgatatttctttcactttacctaaagctgaaggcagaaaagccgtcgctgcccttgaaaaaacacgagctgctgttggattcgattccttaagatatgatgatcaaattgcaaagattagtttagttggagctggaatgaaaacaaacccaggcgttactgcaacattttttgaagccctctcaaatgctggtgttaatattgagctcatttcaacttcagagattagaatttcagtagttactagagctgatgacgttaatgaagctgttcaagctgttcacagtgcattcggattggattctgagactgacgaagctgttgtgtatggtggtactggtaga

SEQ ID NO: 19 - DNA, 1289 bp, Solanum sp.
ccttaattctatacactattatttcctcttttattctacatttcattcttagcttattttttcgtaaacgttacccagtgccactaccacccggtccaaaaccatggccaataatcggaaacatagtccaattaggtccgaagccgcaccagtccactgcatcaatggcccgaacttacgggccactcatgcaccttcgcatggggttcgtggacgtggtggttgcggcctcagcttcggtggcggctcaatttttgaaaaatcatgacgctaacttctcgagccgcccaccgaactctggggcgaaacacatggcttataattaccatgaccttgtgtttgcaccttacggaccacggtggcgtatgctaaggaaaatttgttctgttcatctcttttcggctaaagctttagatgacttccgccatgtccgacaggaagaagtcagaacacttacgcgcgccttagcaaatgctggccaaaagccaatcaaattagggcagctgttgaacgtgtgcaccacgaatgcacttgcgcgtgtgatgctcgggaagcgggtattcgccgacggtactaacggtatcgatccacaagcggaggagttcaagttaatggtggtggagatgatggtgctcgccggcgttttcacatcggcgattttattccggcgcttgattggatggacattcaaggcgtagcaggaaaaatgaagaaactccacgcgcgtttcgacgcgttcttaaccacgatcctcgaagaacacaagggaaagcgagttggagaatcgaaggagcagggggatttgttgaatacgttgatctctctgaaaaatgaagaagacgataatggaggaaagcttactgatacagaaattaaagctttactttgggtacgcctcttacaattatctctttatttcaaattggacaagtaaaaacaaatatggatttttagtatatctaacaagtaaaaaggaatagaggtaataaatatgaaactatgccatttttctttgacggactaaaaatggaagtatgctaatgtcctaatttatatgataatgtttggcttgaaacaatgttgtttaagaagttaatttttattcgtcctgcaattttaatggtatgagttcgaatttcaggatatagtttgatcaattgttcttatacaaattcactctaatattacaaacttacaaatttgaagtttaaagatttatcagttcaaatttcatgatttttcaccttttcaaagccttaaactcgaattatacaagtgtgggagttatt

SEQ ID NO: 20 - DNA, 2517 bp, Triticum sp.
atggctaagcggttagtcctctttgtggcggtagtcgtcgccctcgtggctctcaccgtcgctgaaggtgaggcctctgagcaactacagtgtgagcgcgagctccaggagctccaggagcgcgagctcaaggcatgccagcaggtcatggaccagcagctccgagacattagccccgagtgccaccccgtcgtcgtcagcccggtcgcgggacaatacgagcagcaaatcgtggtgcccaagggcggatctttctaccccggcgagaccacgccaccgcagcaactccaacaacgtatattttggggaatacctgcactactaaaaaggtattacccaagtgtaacttctccgcagcaggtttcatactatccaggccaagcttctccgcaacggccaggacaaggtcagcagccaggacaagggcaacaatcaggacaaggacaacaagggtactacccaacttctccgcaacagccaggacaatggcaacaaccggaacaagggcaaccagggtactacccaacttctccgcagcagccaggacaattgcaacaaccagcacaagggcagcaaccaggacaaggacaacaaggtcggcagccaggacaagggcaaccagggtactacccaacttcttcgcagctgcagccaggacaattgcaacaaccagcacaagggcaacaagggcagcaaccaggacaagggcaacaaggtcaacagccaggacaagggcaacaaccaggacaaggacaacaaggtcaacagccaggacaagggcaacaaccaggacaagggcaacaaggtcagcagctcggacaaggacaacaagggtactacccaacttctctgcaacagtcgggacaagggcaaccagggtactacccaacttctctgcagcagctaggacaagggcaatcagggtactacccaacttctccgcagcaaccaggacaagggcagcagccaggacaattgcaacaaccagcacaagggcagcaaccagaacaagggcaacaaggtcagcagccaggacaagggcaacaaggccagcagccaggacaagggcagcaaccgggacaagggcaaccagggtactacccaacttctccgcagcagtcaggacaagggcaaccagggtactacccaacttcttcgcagcagccaacacaatcgcagcaaccaggacaagggcaacaaggtcagcaggtaggacaagggcaacaagctcagcagccaggacaagggcagcaaccgggacaagggcagccagggtactacccaacttctccgctgcagtcaggacaagggcaaccagggtactacctaacttctccgcagcagtcaggacaagggcagcagccaggacaattgcaacaatcagcacaagggcaaaaaggacagcaaccaggacaaggtcaacagccagggcaagggcaacaaggtcagcagccaggacaagggcaacaaggtcagcaaccggggcaagggcagccagggtactacccaacttctccgcagcaatcaggacaagggcaacagccaggacaatggcaacaaccaggacaagggcaaccaggatactacccaacttctccgttgcagccaggacaagggcaaccagggtacgacccaacttctccgcaacagccaggacaagggcagcaaccaggacaattgcaacaaccagcacaagggcaacaagggcagcaactagcacaagggcaacaagggcagcaaccagcacaagtgcaacaagggcagcagccagcacaagggcaacaaggtcagcagctaggacaagggcaacaaggtcagcagccaggacaagggcagcaaccagcacaagggcaacaaggtcagcagccaggacaagggcaacaaggtcagcagccaggacaagggcagcaaccgggacaagggcagccatggtactacccaacttctccgcaggagtcaggacaagggcaacagccaggacaatggcaacaaccaggacaatggcaacaaccaggacaagggcaaccagggtactacctaacttctccgttgcagctaggacaagggcaacaagggtactacccaacttctctgcaacaaccaggacaagggcagcaaccaggacaatggcaacaatcgggacaagggcaacatgggtactacccaacttctccgcagctgtcaggacaagggcaacggccaggacaatggctgcaaccaggacaagggcaacaagggtactacccaacttctccgcaacagtcaggacaagggcaacaactaggacaatggctgcaaccaggacaagggcaacaagggtactacccaacttctctgcaacagacaggacaagggcagcaatcaggacaagggcaacaaggctactacagctcataccatgttagcgtggagcaccaggcggccagcctaaaggtggcaaaggcgcagcagctcgcggcacagctgccggcaatgtgccggctggagggcggcgacgcattgtcggccagccagtga

SEQ ID NO: 21 - DNA, 154 bp, Artificial Sequence
  Description of Artificial Sequence Synthetic polynucleotide
ttagtctctattgaatctgctgagattacactttgatggatgatgctctgtttttgttttcttgttctgttttttcctctgttgaaatcagctttgttgcttgatttcattgaagttgttattcaagaataaatcagttacaattatgtttggg

SEQ ID NO: 22 - DNA, 179 bp, Artificial Sequence
  Description of Artificial Sequence Synthetic polynucleotide
accttatttcactaccactttccactctccaatccccatactctctgctccaatcttcattttgcttcgtgaattcatcttcatcgaatttctcgacgcttcttcgctaatttcctcgttacttcactaaaaatcgacgtttctagctgaacttgagtgaattaagccagtgggaggat

SEQ ID NO: 23 - DNA, 273 bp, Artificial Sequence
  Description of Artificial Sequence Synthetic polynucleotide
ttagagtgtgggtaagtaattaagttagggatttgtgggaaatggacaaatataagagagtgcaggggagtagtgcaggagattttcgtgcttttattgataaataaaaaaagggtgacatttaatttccacaagaggacgcaacacaacacacttaattcctgtgtgtgaatcaataattgacttctccaatcttcatcaataaaataattcacaatcctcactctcttatcactctcattcgaaaagctagatttgcatagagagcacaaa

SEQ ID NO: 24 - DNA, 1659 bp, Solanum sp.
gttcatcttcttctctcacttctcttaacaacatcttttctgcattggccacttagttggttaggaggtgaacatggctcagattctggctccatctgcacaatggcagatgagaatgacaaagagctcaacagatgctagtcccttgacttcaaagatgtggagctctgtggtgctgaagcagaacaaaagacttgctgttaaaagctctgccaaatttagagtttttgccctccagtctgacaatggcaccgtgaacagaatggaacagctgctaaacttggacgtaactccatacactgataagatcattgctgaatatatttggatcggggggactggaattgatgtgcgcagtaaatcaaggactatttcaaaaccagtcaaggatgcttctgagctcccaaagtggaactacgatggatcaagtactggacaagcacctggagaagacagtgaagtcattctatatcctcaggcaatattcaaagaccctttccgtggtggtaacaacatcttggttatctgtgatacctacacaccagctggagagccaattcctacaaacaaacgccataaagctgctcaaatttttagcgacccaaaagttgcatctcaagttccatggtttggaatagaacaagagtacaccttactccagccaaatgttaactggcccttaggttggcctgttggaggctatcctggcccactaggttcttactactgtggtgctggagtggaaaagtcatttggccgagatatatcagatgctcactacaaggcttgcctgtatgctggaattaacattagtggtactaacggagaggttatgccaggacagtgggaatttcaagtaggacctagtgttggaattgaaggtggagatcatatctggtgtgctagatacctcctcgagagaattactgaacaagcaggagttgtcctctcactcgatccaaaaccaattgagggtgactggaacggtgcaggatgccacactaactacagtacactgagtattagagaagagggaggctttgaagtgataaagaaagcaattcttaatctatccctgcgccacaagggacatataagtgcttatggagaaggaaatgagagaaggttgaccggaaagcatgaaactgctagtattgaccaattttcatggggagttgctaaccgtggttgctcaatccgtgtggggcgttacactgaaaaagaagccaggggttatttggaagaccgccgcccagcttcaaacatggacccctatgttgtgaccgcattacttgccgaaactactatactgtgggagccaacccttgaggctgaagctcttgctgcccaaaagatctcattgaaggtttagagtaattgaggggaaatgttttcatcataatccttcttagaatttatgagataagtgtgaagcttgtaccttgttgagattcccttatttgggaaattcttgtaaaggaattcaaaatttaaccagttcatcctagaaagaggttccttaagacatgagactactttggagttgaggtgtaattgttggactactttgaacatctttacctttcttttctccagatgaatccatttctctgaaattccaattggtgtgatttttccgaaaaaaaaaaaaaaaaaaaa

SEQ ID NO: 25 - DNA, 1400 bp, Solanum sp.
gatcaaattaattaaattctctcattcaacaaattcaaactttagaattattattattttcacataatggctcatctttcagatcttgtcaatctcaatctctctgattcctctgacaaaatcattgctgaatacatatggattggtggatcaggaatggatgtaaggagcaaagccaggactctatctggtcctgttgatgatccttcaaagcttcccaaatggaattatgatggttctagcacaggtcaagctcctggagaagacagtgaagtgatcctatatcctcaagcaattttcaaggatccattcaggaggggcaacaatatcctggtcatctgtgattgttacaccccagctggtgaaccaattccaacaaacaagatgtgtcacaatgctgctaaaatatttagtaaccctgatgttgttgttgaggaaccatggtatggtcttgaagcagaatacaccttgctacaaagaaggaattactggcctcttggatggcctattggtggttttcctggattacagggaccatactactgtggaattggatctggaaaggcttttggacgcgatattgttgatgctcattacaaggcatatatctatgccgggattaacattagtggtatcaacggagaagtgatgcctggacagtgggaatttcaagttggtcttcaagttggcattgcatcaggtgacgaagtgtgggcagctcgttacattctcgagaggattacagagattgctggagttgtcgtgtcattcgaccccaaacctattccgggcgactggaatggtgcaggagcgcatacaaattacagtaccaagtccatgaggaatgagggagggtatgaagttatcaagaaggctattgagaagcttggacttaggcacaaggagcacattgcagcatatggtgaaggcaatgaacgtcgtctcactggaaagcacgaaacagctgacatcaacacgttcaaatggggtgttgcaaatcgtggtgcatccattcgtgtgggaagagacacggagaaggaaggcaagggatactttgaggacaggaggcctgcatcgaacatggatccatacatcgtgacctctatgtacgcggagactaccctcctgtggaacccttgaacgcgtatgggatgaatattctcgggggtgcaaacatatgaggaaagaattgaatttcttaacaggccctttcctcacatgtccttaagagagttatgtagctagtaattttgatatattatgttgttttctaagtttcaatttgtattgtactcagcaagcctgagttcattggccaaaatcgatttggcaatgttgttaaaaataagagttttaatcttattaataacaatatggaagggtttaacttaaaaaaaaaa

SEQ ID NO: 26 - DNA, 1397 bp, Solanum sp.
gatcttatagagaatttcaattttaagaagttatcatcatgtctctgctttcagatcttatcaacctcaatctctcagatgatactcagaagatcattgctgaatacatatggattggtggatcaggcatggacatgaggagcaaagccaggactctccctggtccagttactagtcctgcagaactacccaaatggaactatgatggatcaagcacaggtcaagctcctggagaagacagtgaagtgatcatatacccacaagcaatcttcaaggatccattcaggagaggcaacaatatcttggtcatgtgtgatgcctatactcctgctggtgagcccatcccaacaaacaagaggcacgccgctgccaaggtcttctgccaccctgatgtggctgctgaggaaacttggtatggtattgaacaagaatataccttgctgcaaaaggaggtcaactggcctcttggatggcccattggcggttttcctggaccccagggaccatactactgtggaactggagctgacaaggcctttggacgtgacattgtggacgcccattacaaggcatgtctctatgctgggattaatatcagcggaatcaatggtgaagtcatgccgggacagtgggaattccaagtgggaccttctgttggcatctcagccggtgatgaagtgtgggtagctcgttacattctagagaggattgcagagattgctggggtggtcgtgtcattcgaccccaagcctattccgggcgactggaacggcgcaggtgctcacacaaattacagcaccaagtcgatgagggaagacggaggctataaaataatcttgaaggctattgagaagcttggcctgaagcacaaagaacacattgctgcatatggtgaaggcaatgagcgtcgtctcactggaaagcacgaaacagccaacatcaacaccttcaaatggggggttgcaaaccgtggtgcatctgtccgtgttggaagagacacagagaaggcaggcaagggatactttgaggacagaaaatatggacccatacgtcgttacctccatgatcgcagaaaccaccatcatcggttaaccttgaagacattttactatggatggctcgggggatcgcttgtttctggtttgcacaatttgggataggagaaaagattgaattgtgaaacgaccctttcgacttcacctgtgttaatttttagttataggggtagattgtctcttgttatttttctgtttatttgccagttgaattgtattttcatacagcaaggccttatacattgtctatgatttggcaatgctgtgttacaaaacaatgttattcttattaataacaaagataatgaaaggatttgattctattgctcaaaaaaaaaaaaa

SEQ ID NO: 27 - DNA, 876 bp, Artificial Sequence
  Description of Artificial Sequence Synthetic polynucleotide
gtttgtaggaattggctctccagctggtgtgtaggtatcacagataaccaagatgttgttaccaccacggaaagggtctttgaatattgcctgaggatatagaatgacttcactgtcttctccaggtgcttgtccagtacttgatccatcgtagttccactttgggagctcagaagcatccttgactggttttgaaatagtccttgatttactgcgcacatcaattccagtccccccgatccaaatatattcagcaatgatcttatcagtgtatggagttacgtccaagtttagcagctgttccattctgttcacggtgccattgtcagactggagggcaaaaactctaaatttggcagagcttttaacagcaagtcttttgttctgcttcagcaccacagagctccacatctttgaagtcaagggactagcatctgttgagctctttgtcattctcatctgccattgtgcagatggagccagaatctgagccatctgtcctcaaagtatcccttgcctgccttctctgtgtctcttccaacacggacagatgcaccacggtttgcaaccccccatttgaaggtgttgatgttggctgtttcgtgctttccagtgagacgacgctcattgccttcaccatatgcagcaatgtgttctttgtgcttcaggccaagcttctcaatagccttcaagattattttatagcctccgtcttccctcatcgacttggtgctgtaatttgtgtgagcacctgcgccgttccagtcgcccggaataggcttggggtcgaatgacacgaccaccccagcaatctctgcaatcctctctagaatgtaacgagctacccacacttcatcaccggctgagatgccaaca

SEQ ID NO: 28 - DNA, 1148 bp, Artificial Sequence
  Description of Artificial Sequence Synthetic polynucleotide
gccaccctgatgtggctgctgaggaaacttggtatggtattgaacaagaatataccttgctgcaaaaggaggtcaactggcctcttggatggcccattggcggttttcctggaccccagggaccatactactgtggaactggagctgacaaggcctttggacgtgacattgtggacgcccattacaaggcatgtctctatgctgggattaatatcagcggaatcaatggtgaagtcatgccgggacagtgggaattccaagtgggaccttctgttggcatctcagccggtgatgaagtgtgggtagctcgttacattctagagaggattgcagagattgctggggtggtcgtgtcattcgaccccaagcctattccgggcgactggaacggcgcaggtgctcacacaaattacagcaccaagtcgatgagggaagacggaggctataaaataatcttgaaggctattgagaagcttggcctgaagcacaaagaacacattgctgcatatggtgaaggcaatgagcgtcgtctcactggaaagcacgaaacagccaacatcaacaccttcaaatggggggttgcaaaccgtggtgcatctgtccgtgttggaagagacacagagaaggcaggcaagggatactttgaggacagaatggctcagattctggctccatctgcacaatggcagatgagaatgacaaagagctcaacagatgctagtcccttgacttcaaagatgtggagctctgtggtgctgaagcagaacaaaagacttgctgttaaaagctctgccaaatttagagtttttgccctccagtctgacaatggcaccgtgaacagaatggaacagctgctaaacttggacgtaactccatacactgataagatcattgctgaatatatttggatcggggggactggaattgatgtgcgcagtaaatcaaggactatttcaaaaccagtcaaggatgcttctgagctcccaaagtggaactacgatggatcaagtactggacaagcacctggagaagacagtgaagtcattctatatcctcaggcaatattcaaagaccctttccgtggtggtaacaacatcttggttatctgtgatacctacacaccagctggagagccaattcctacaaac

SEQ ID NO: 29 - DNA, 1197 bp, Escherichia coli
ccaggcgactgtcttcaatattacagccgcaactactgacatgacgggtgatggtgttcacaattccacggcgatcggcacccaacgcagtgatcaccagataatgtgttgcgatgacagtgtcaaactggttattcctttaaggggtgagttgttcttaaggaaagcataaaaaaaacatgcatacaacaatcagaacggttctgtctgcttgcttttaatgccataccaaacgtaccattgagacacttgtttgcacagaggatggcccatgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacgactaacgtcgcagcgcgtgatatggcccagatgtgcaaactggcagcagaagaacattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaaagtttagggagatttgatggcttactctgttcaaaagtcgcgcctgg

SEQ ID NO: 30 - DNA, 903 bp, Corynebacterium glutamicum
atgagcacaggtttaacagctaagaccggagtagagcacttcggcaccgttggagtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgagtctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgcttcgcgtctgcagggcatcaacgtaggagatcctcgacttccaattatggctccaaatgagcaggaacttgaggctctccgagaagacatgaaaaaagctggagttcta

SEQ ID NO: 31 - DNA, 1140 bp, Artificial Sequence
  Description of Artificial Sequence Synthetic polynucleotide
atgatttcgccgacgaatctcctcccggcgcggaagatcacccctgtctcaaatggcggcgcagcgacggcgagcccctcttctccctcggtggccgcacggccacggcgactcccttcaggcctccaatctgtgactggtagagggaaggtttccttggcagccatcactctagatgattaccttccaatgcgaagcactgaagtgaagaaccggacatcaacagatgacatcacaaggctgagactaatcacagcagttaaaaccccctatttgccagatgggaggttcgatctggaagcatatgattctctcataaacatgcagatagagggtggtgccgaaggcgtaatagttggaggaacaacaggagagggtcacctcatgagctgggacgaacatatcatgctcattgggcacacagtgaactgctttggctctagaatcaaagtgataggcaacacaggaaacaactcaaccagagaagccgtccacgcaacagaacagggatttgctgttggcatgcatgcggctctccacatcaatccttactacgggaagacctcagctgaaggaatgatctctcatttcgaggctgtcctcccgatgggtccgaccatcatctacaacgtgccatccaggagcgcccaggacatcccccctgaagttattctagcgatttctggctacacaaacatggcgggtgtcaaggaatgtgttgggcacgagagggttaagcactacgctgacaaaggcataacaatttggagcggtaacgacgacgagtgccacgattctaagtggaaacatggcgctactggggtcatttccgttaccagcaaccttgttcccgggctcatgcacagcctcatgtacaaaggcgagaacgccacgctgaacgagaagctgtcgcccctgatgaaatggctgttctgccagccaaatcctattgccctcaacactgctctggctcagctcggcgtggcaaggcctgtcttcagactgccgtacgttccgctccctcttgaaaagagggccgagttcgtccggattgttgagtcaattggacgggaaaatttcgtggggcagaaggaggcaccggttctagatgatgacgatttcgtgttgatcagtaggtac

SEQ ID NO: 32 - DNA, 1140 bp, Artificial Sequence; identical to SEQ ID NO: 31
  Description of Artificial Sequence Synthetic polynucleotide

SEQ ID NO: 33 - DNA, 1140 bp, Artificial Sequence; identical to SEQ ID NO: 31
  Description of Artificial Sequence Synthetic polynucleotide

SEQ ID NO: 34 - DNA, 1583 bp, Solanum tuberosum
gatcaagcccagaaggataggattacaagtgttgagggaaaaatgtcatcatcagttataggtcgctgccatttcggcgttgaagctgccggaacaaagaggagaactgcagtatggaggtcaccgagagcagctgtaattccaagtttccacctcccaatgcgtagttatgaagttaaaaataggacttttgccgaggatataaaagcacttcggttgattacctgcaatcaaaaccccatatctaccagatggaagattcgatcttgaggcatatgacgccttggtaaatttgcaaattgaaaatggcgctgagggtgtgattgttggtggcactaccggtgaaggtcaattgatgaagctgggatgaacacatcatgctcattggtcacacagtgaactgttttggaggatcaatcaaagtcattgggaacacaggaagtaactccacgagggaagcaatccatgcgacagaacagggatttgctgtaggtatgcatgcagctcttcacattaatccctactatgggaagacctccatagagggtatgatttcacactttgaaagtgtgctccctatgggccctactatcatttataacgtgccatcaagaactggtcaagacattcctccacgtgtaattcagacattagcagagagccctaatcttgccggtgtcaaagagtgtgttggaaatgatagagtgaagcagtatacaagtaatggtttggtcgtgtggagtgggaatgatgatgaatgccacgactcaaggtgggatcatggtgctgcaggagttatttctgttaccagcaacttggttcctggtctgatgcgtgaactaatgtttggaggaaagaaccctgctttgaactcaaaggtaatgccattggttgaatggttgtttcatgaaccaaaccccattgctctaaatacagctttagctcaactcggagttgtgaggcctgtcttccgccttccatatgttccacttacaagggcaaagagagaggtgtttgtgaagattgtcaaggaaatcggacgggaaaactttataggggaaagagatgtccaagttttggatgatgatgattttgttttgcttggtaggtattagcttttccccatttggtgttgagaagtaagtttttgttgttagctgcttaatttgaaaatttcttaacatgaagaaatatatcttcttttatgttcttttttgtaaccgtgcatttttcttgtttgctcttctcttctaggaggcatgtacattgtagctgttagtttatggttctaaactaaagatgtgcataatgtatcaaaaaaaaaaaaaacggcacgaggtagcctcaaaatttcaactttatatttttgctatttattgggccgaaaccccccttctattataattattatcttgtcagttacttaacgaacaggtggcaacacttgggtctaagggccaaaaagaaaatatttcttttatgtttagactaaggaccatgtaccatggattaaattggcccaaaaccaaaaccaataggcccagtttggccaggcgaacaaaaatctggttgggcccagcccc

SEQ ID NO: 35 - DNA, 1481 bp, Triticum aestivum
ccaaaccaaagccctaaatccctctttcggaagataggagccgccccctttcgtcgtcgccgccatcgccgttctacagctgccgccatgccgtacctccagcccccgcgcccccacccccacccccacccgacctcccgcctctcgcgcgcgtcgccgccgtcgccgttcccgttcttccccgccgggacatcgcgctccggtcgtctccaacccgtcccagtctccggccactcggcctcgagggttagtaaaggaaagtttgcagttgcagccgtcactctagatgattatcttccgatgcgaagtactgaagtgaaaaaccggacatcaacagatggaatcaaaagcctcagactaatcacagcggtcaaaaccccctatttgccagatggaagatttgatcttgaagcatacgattctctgataaacacgcaaataaatggcggtgcggaaggtgtaatagttggaggaacaacaggagaaggtcacctcatgagctgggatgagcacatcatgctcatcgggcatactgttaactgctttggagccaacattaaagtgataggcaacacgggaagtaactcaaccagagaagctgttcacgcgacagagcagggatttgctgttggcatgcatgcagctcttcatgtcaatccttactacgggaagacctcaactgaaggcctgatctctcattttaaggaagtcctcccgatgggcccaacaatcatttacaacgtgccatccaggacaagtcaggacatacctccaccagtcattgaggcgctttcatgttactcaaacatggcaggagtgaaagaatgcgttgggcatgagcgggttaagtgctacactgacaaaggcataacaatatggagcggcaacgatgatgaatgccatgactcaaggtggaagtacggcgctactggagtcatttctgtggcgagcaaccttgttcccggtctcatgcacagcctcatgtttgaaggggagaatgcggcgctaaatgagaagctgctgcctctgatgaaatggctgttctgcgagcctaacccgatcggtctcaacaccgccctggctcagctcggtgtagtgaggcctgttttcaggttgccatacactcccctccctcttgagaagagggttgagtttgtccggattgtcgaagccattggacgggagaactttgtgggacagaaggaggcccgggttctggatgacgatgatttcgtgttgatcagcaggtattgaatatgtgtgtgagttctttgtgtccagtgtatgctgacgaacagcggtagtgtcacagtgttgtttgcggatcgttttagacgcttcgtttgttcaccagtcatttgttgatctttttctctgactgcatgttaatttggaacccgtttggtttattggcaccttgtgataccagttccagtaagaaatgagaatagcccgagacatgtacagtcgggttgttacaa

SEQ ID NO: 36 - DNA, 1396 bp, Solanum sp.
gcacacagtaacacaccacaactcctaatttattttctccgccgtagcttttaccttttcctcatttcttttgtatattccgaaatgaggacctccatgttgaaatccatcgtccgccggagctccactgccggagcatcctatgtatcccgccgtggattcgcatcgggatccgcaccagagaggaaagttgcagttttgggggcagccggaggaattggacagcctttatctcttctaatgaagcttaaccctttagtatccagcctttcactctacgatatcgccggcactcccggtgttgccgccgatgttagtcacatcaacaccagatctgaggttgtcggttttgcaggagaagagcagctagggaaggctctggaaggagctgatatagttatcattcctgctggtgtgccccgaaagcctggtatgacccgagatgatctgttcaacattaatgcgggtattgttaaatctctttgcacggccattgctaagtactgccccaatgctctggtcaatatgataagcaacccagtgaattccactgtcccaattgctgctgaggtgtttaagaaggctggaacttatgatgaaaagaagctctttggagttaccatgcttgatgtggttagggccaagacattttatgctggaaaagctaaagtaaatgttgctgaggtcaatcttccggtagttggtggtcatgctggcataactatcctcccattattttctcaagccactccaaaggcaaatctgtcaaatgaggaaattgttgccctcacaaagcgaacacaagatggtggcacagaagtcgtggaggccaaggctggaaagggttcagccaccctctcaatggcctatgctggggctatttttgccgatgcttgcttgaagggtttgaatggagttcccgatgttgttgaatgtgcttttgtgcagtccaatgttaccgagcttcccttctttgcatccaaggtgagacttgggaaaaatggagtggaggaagtccttgggttgggtccacttaacgagtatgagaagcaaggacttgaggctctgaagccagagctgctctcctccattgaaaagggaatcaagtttgccaaagagaactaaaaacaaaattatggtgtagttttctagagtgacagttttggatccttttgtgcaattgtgttgtatcctttgcaagtttcttgcagccggaggcttagatttagctcttttgatatatacccaacatttctacaaaataatgtatggcaaactggcgtctatgtcatttgccttagtgtggaggtgttattctcacatggatcgttttccaattatggttagaaagcagacaattgatttgaaatgaagaatgttaatgaccaattactgt

SEQ ID NO: 37 - DNA, 895 bp, Artificial Sequence
  Description of Artificial Sequence Synthetic polynucleotide
ttcactctacgatatcgccggcactcccggtgttgccgccgatgttagtcacatcaacaccagatctgaggttgtcggttttgcaggagaagagcagctagggaaggctctggaaggagctgatatagttatcattcctgctggtgtgccccgaaagcctggtatgacccgagatgatctgttcaacattaatgcgggtattgttaaatctctttgcacggccattgctaagtactgccccaatgctctggtcaatatgataagcaacccagtgaattccactgtcccaattgctgctgaggtgtttaagaaggctggaacttatgatgaaaagaagctctttggagttaccatgcttgatgtggttagggccaagacattttatgctggaaaagctaaagtaaatgttgctgaggtcaatcttccggtagttggtggtcatgctggcataactatcctcccattattttctcaagccactccaaaggcaaatctgtcaaatgaggaaattgttgccctcacaaagcgaacacaagatggtggcacagaagtcgtggaggccaaggctggaaagggttcagccaccctctcaatggcctatgctggggctatttttgccgatgcttgcttgaagggtttgaatggagttcccgatgttgttgaatgtgcttttgtgcagtccaatgttaccgagcttcccttctttgcatccaaggtgagacttgggaaaaatggagtggaggaagtccttgggttgggtccacttaacgagtatgagaagcaaggacttgaggctctgaagccagagctgctctcctccattgaaaagggaatcaagtttgccaaagagaactaaaaacaaaattatggtgtagttttctagagtgacagtttt

SEQ ID NO: 38 - DNA, 1164 bp, Artificial Sequence
  Description of Artificial Sequence Synthetic polynucleotide
aaaactgtcactctagaaaactacaccataattttgtttttagttctctttggcaaacttgattcccttttcaatggaggagagcagctctggcttcagagcctcaagtccttgcttctcatactcgttaagtggacccaacccaaggacttcctccactccatttttcccaagtctcaccttggatgcaaagaagggaagctcggtaacattggactgcacaaaagcacattcaacaacatcgggaactccattcaaacccttcaagcaagcatcggcaaaaatagccccagcataggccattgagagggtggctgaaccctttccagccttggcctccacgacttctgtgccaccatcttgtgttcgctttgtgagggcaacaatttcctcatttgacagatttgcctttggagtggcttgagaaaataatgggaggatagttatgccagcatgaccaccaactaccggaagattgacctcagcaacatttactttagcttttccagcataaaatgtcttggccctaaccacatcaagcatggtaactccaaagagcttcttttcatcataagttccagccttcttaaacacctcagcagcaattgggacagtggaattcactgggttgcttatcatattgaccagagcattggggcagtacttagcaatggccgtgcaaagagatttaacaatacccgcattaatgttgaacagatcatctcgggtcataccaggctttcggggcacaccagcaggaatgataactatatcagctccttccagagccttccctagctgctcttctcctgcaaaaccgacaacctcagatctggtgttgatgtgactaacatcggcggcaacaccgggagtgccggcgatatcgtagagtgaaaggctggatactaaagggttaagcttcattagaagagataaaggctgtccaattcctccggctgcccccaaaactgcaactttcctctctggtgcggatcacgatgcgaatccacggcgggatacataggatgctccggcagtggagctccggcggacgatggatttcaacatggaggtcctcatttcggaatatacaaaagaaatgaggaaaaggtaaaagctacggcggagaaaataaattaggagttgtggtgtgttactgtgtgc